(19) United States
(12) Patent Application Publication
(10) Pub. No.: US 2025/0340668 A1
(43) Pub. Date: Nov. 6, 2025

(54) MULTISPECIFIC BINDING MOLECULE PROPROTEINS AND USES THEREOF

(71) Applicant: Regeneron Pharmaceuticals, Inc., Tarrytown, NY (US)

(72) Inventors: Lauric HABER, Rye Brook, NY (US); Alison CRAWFORD, Dobbs Ferry, NY (US); Tong ZHANG, New Rochelle, NY (US); Eric SMITH, New York, NY (US); Chia-Yang LIN, Scarsdale, NY (US)

(73) Assignee: Regeneron Pharmaceuticals, Inc., Tarrytown, NY (US)

(21) Appl. No.: 18/864,231

(22) PCT Filed: May 10, 2023
(86) PCT No.: PCT/US2023/066841

§ 371 Date: Nov. 8, 2024

Related U.S. Application Data
(60) Provisional application No. 63/340,891, filed on May 11, 2022.
(60) Provisional application No. 63/481,291, filed on Jan. 24, 2023.
(60) Provisional application No. 63/489,812, filed on Mar. 13, 2023.

(51) Int. Cl. C07K 16/30; C07K 16/28

(52) U.S. Cl. CPC C07K 16/30; C07K 16/2809; C07K 2317/31; C07K 2319/50

(57) ABSTRACT

The present disclosure provides proproteins of antigen-binding molecules, including multispecific binding molecules (MBMs). The antigen-binding molecule proproteins comprise an antigen-binding site, one or more components that inhibit the binding of the antigen-binding site to its target, and a protease-cleavable linker that can be cleaved by a protease and whose cleavage removes the inhibition of binding of the antigen-binding site to its target. The MBM proproteins and MBMs resulting from cleavage of a protease-cleavable linker. e.g., tandem Fab MBMs, comprise a T-cell engaging antigen-binding site and a tumor-associated antigen antigen-binding site. The MBM proproteins further comprise a component that inhibits the binding of the T-cell engaging antigen-binding site to its target and a protease-cleavable linker that can be cleaved by a protease in the tumor environment and whose cleavage removes the inhibition of binding of the T-cell engaging antigen-binding site to its target. The disclosure further provides pharmaceutical compositions comprising the antigen-binding molecule proproteins, and methods of use of the antigen-binding molecule proproteins in therapy, as well as nucleic acids encoding the antigen-binding molecule proproteins, recombinant cells that express the antigen-binding molecule proproteins, and methods of producing the antigen-binding molecule proproteins.

### 1. CROSS-REFERENCE TO RELATED APPLICATIONS

This application claims the priority benefit of U.S. provisional application No. 63/340,891, filed May 11, 2022, U.S. provisional application No. 63/481,291, filed Jan. 24, 2023, and U.S. provisional application No. 63/489,812, filed Mar. 13, 2023, the contents of each of which are incorporated herein in their entireties by reference thereto.

### 2. SEQUENCE LISTING

The instant application contains a Sequence Listing which has been submitted electronically in XML file format and is hereby incorporated by reference in its entirety. Said XML copy, created on May 9, 2023, is named RGN-017WO_SL.xml and is 307,242 bytes in size.

### 3. BACKGROUND

The selective destruction of tumor cells, while leaving healthy cells and tissues intact and undamaged, is a goal of cancer immunotherapy. Bispecific antibody therapeutics have been developed to achieve this goal by inducing an immune response against the tumor. In this regard, bispecific antibodies are designed to both a tumor-associated antigen (“TAA”) expressed preferentially on tumor cells and a component of the T cell receptor (“TCR”) complex. The simultaneous binding of such an antibody to both of its targets results in activation of cytotoxic T cells and subsequent lysis of the TAA-expressing cell. Hence, the immune response is re-directed to the TAA-expressing cells.

Several bispecific antibody formats have been developed and their suitability for T cell mediated immunotherapy investigated. See You et al., 2021, Vaccines 9:724, doi.org/10.3390/vaccines9070724.

The task of generating bispecific molecules suitable for treatment provides several technical challenges related to toxicity, as TAAs are typically expressed on normal cells as well as tumor cells. There is thus a need for efficacious T-cell activating bispecific molecules that unleash T-cell activation selectively in the tumor environment.

### 4. SUMMARY

The present invention generally relates to novel protease-activatable antigen-binding molecules (“ABMs”), referred to herein as antigen-binding molecule proproteins.

Generally, in their active state, the antigen-binding molecules comprise an antigen-binding site (ABS) that can bind to its target. However, in proprotein form, the ABS is masked by a masking moiety such that its ability to bind to its target is greatly diminished. The antigen-binding molecule proproteins are configured such that upon encountering a protease, e.g., a protease that is overexpressed in the tumor environment, the masking moiety is cleaved and a binding molecule is produced having enhanced target binding. This is achieved through the inclusion of one or more protease-cleavable linkers (“PCLs”) that comprise one or more protease substrate sequences, e.g., directly or indirectly between the ABS and the masking moiety. Proproteins can provide for reduced toxicity and adverse side effects that could otherwise result from binding of an ABM to normal tissues. Generally, antigen-binding molecule proproteins are described in Section 6.2 and Group A numbered embodiments 1 to 43, as well as Group B numbered embodiments 1 to 90.

In some embodiments, an antigen-binding molecule proprotein of the disclosure is a multispecific binding molecule (MBM) proprotein. Generally, in their active state, MBMs of the disclosure comprise a T-cell engaging antigen-binding site that binds to a component of the T-cell receptor complex (a “TCE ABS”) and a tumor-associated antigen-binding site (“TAA ABS”) that can simultaneously bind to their respective targets such that the T-cell bound by the TCE ABS is stimulated to attack the TAA-expressing tumor cell bound by the TAA ABS. However, when the MBM is in proprotein form, the TCE ABS is masked by a masking moiety such that its ability to bind its target is greatly diminished. The MBM proprotein is configured such that upon encountering a protease, e.g., a protease that is overexpressed in the tumor environment, the masking moiety is cleaved and the inhibition of TCE ABS binding is reversed. This is achieved through the inclusion of protease-cleavable linkers (“PCLs”) that comprise one or more protease substrate sequences in the MBM proprotein, e.g., directly or indirectly between the masking moiety and the TCE ABS. Proproteins can provide for reduced toxicity and adverse side effects that could otherwise result from binding of an MBM to normal tissues. Generally, MBM proproteins are described in Section 6.3 and Group B numbered embodiments 1 to 5, 15, 54, and 69 to 72.

In certain aspects, the MBM proproteins (referred to as Type I MBMs) comprise an anti-idiotype of the TCE ABS which reversibly masks binding of the TCE ABS to its target, referred to herein as an “anti-TCE ABS”. Thus, type I MBMs comprise anti-TCE ABS masking moieties. In some embodiments, the MBM proprotein is configured such that activation of the MBM proprotein releases an MBM (e.g., a bispecific binding molecule) comprising a full Fc region. This type of MBM proprotein is referred to as Type IA MBM proprotein and exemplary embodiments of Type IA MBMs are depicted in FIGS. 1A-1C and described in Section 6.3.1.1 and Group B numbered embodiments 6 to 8 and 18 to 24. In other embodiments, the MBM proprotein is configured such that activation of the MBM proprotein releases an MBM lacking an Fc region, such as a tandem Fab or a BiTe. This type of MBM proprotein is referred to as Type IB MBM proprotein and exemplary embodiments of Type IB MBMs are depicted in FIGS. 5A-5B and described in Section 6.3.1.2 and Group B numbered embodiments 16 and 34 to 40.

In other aspects, the MBM proproteins (referred to as Type II MBMs) the TCE ABS is sterically hindered from binding to its target by virtue of its proximity to an adjacent domain, e.g., an Fc domain, that sterically hinders its binding to its targets. In some embodiments, the MBM proprotein is configured such that activation of the MBM proprotein releases an MBM (e.g., a bispecific binding molecule) comprising a full Fc region. This type of MBM proprotein is referred to as Type IIA MBM proprotein and exemplary embodiments of Type IIA MBMs are depicted in FIGS. 3A-3F and described in Section 6.3.1.1 and Group B numbered embodiments 9 to 14 and 25 to 33. In other embodiments, the MBM proprotein is configured such that activation of the MBM proprotein releases an MBM lacking an Fc region, such as a tandem Fab or a BiTe. This type of MBM proprotein is referred to as Type IIB MBM proprotein and exemplary embodiments of Type IIB MBMs are depicted in FIG. 7 and described in Section 6.3.1.2 and Group B numbered embodiments 17 and 41 to 49.

Section 6.4, Group A numbered embodiments 29 to 31, and Group B numbered embodiments 50 to 53 describe exemplary protease-cleavable linkers that can be used in the antigen-binding molecule proproteins, including MBM proproteins, of the disclosure.

The present disclosure further provides tandem Fab MBMs comprising a TAA ABS and a TCE ABS. The tandem Fab may be produced by the activation of a Type IB or Type IIB MBM as a proprotein, or it can be generated through the expression of the tandem Fab protein (with relevant signal sequences but without a masking moiety). In such instances, the TAA ABS and TCE ABS preferably share a common light chain sequence. Section 6.11 and Group B numbered embodiments 75 to 83 describe exemplary tandem Fab MBMs.

Section 6.5 and Group B numbered embodiment 55 describe exemplary non-cleavable linkers that can be incorporated in the antigen-binding molecule proproteins, including MBM proproteins, and tandem Fab MBMs of the disclosure. Section 6.6, Group A numbered embodiment 23, and Group B numbered embodiments 56 to 64 describe exemplary TAA ABSs that can be incorporated into the antigen-binding molecule proproteins, including MBM proproteins, and tandem Fab MBMs of the disclosure. Section 6.7, Group A numbered embodiment 22, and Group B numbered embodiments 64 to 68 describe exemplary TCE ABSs that can be incorporated in the antigen-binding molecule proproteins, including MBM proproteins, and tandem Fab MBMs of the disclosure. Section 6.8 describes exemplary anti-TCE ABSs that can be incorporated in the antigen-binding molecule proproteins, including MBM proproteins, of the disclosure. Section 6.9 describes suitable formats for the ABSs that are incorporated in the antigen-binding molecule proproteins, including MBM proproteins, of the disclosure. Section 6.10 and Group B numbered embodiments 73 and 74 describe suitable Fc domains that can be incorporated in the antigen-binding molecule proproteins, including MBM proproteins, of the disclosure.

The disclosure further provides nucleic acids encoding the antigen-binding molecule proproteins, including MBM proproteins, and tandem Fab MBMs of the disclosure (either in a single nucleic acid or a plurality of nucleic acids) and recombinant host cells and cell lines engineered to express the nucleic acids and antigen-binding molecule proproteins, including MBM proproteins, of the disclosure. Exemplary nucleic acids, host cells, and cell lines are described in Section 6.12, Group A numbered embodiments 47 and 48, and Group B numbered embodiments 87 to 92.

Pharmaceutical compositions comprising the antigen-binding molecule proproteins, including MBM proproteins, and tandem Fab MBMs of the disclosure are also provided. Examples of pharmaceutical compositions are described in Section 6.13, Group A numbered embodiment 44 and Group B numbered embodiment 84.

Further provided herein are methods of using the antigen-binding molecule proproteins, including MBM proproteins, tandem Fab MBMs, and pharmaceutical compositions of the disclosure, for example for treating proliferative conditions (e.g., cancers), on which the TAAs are expressed. Exemplary methods and indications are described in Section 6.14, Group A numbered embodiments 45 and 46, and Group b numbered embodiments 85 and 86.

### 5. BRIEF DESCRIPTION OF THE FIGURES

FIGS. 1A-1C are illustrations of embodiments of Type IA MBM proproteins, in which a TCE ABS is masked by an anti-TCE ABS. In the embodiments of FIG. 1A and FIG. 1B, the MBMs produced by the cleavage of Linker B are bivalent, with one TAA ABS and one TCE ABS. In the embodiment of FIG. 1C, the MBM produced by the cleavage of Linker B are trivalent, with two TAA ABSs and one TCE ABS. In some embodiments, the trivalent MBMs are bispecific. Although the ABSs in FIGS. 1A-1C are illustrated as Fabs, the ABSs can be in other formats, e.g., scFvs or other formats described in Section 6.9. In all embodiments depicted in this figure, an MBM comprising the Fc domains is released following cleavage of Linker B.

FIG. 2 illustrates the mechanism of activation of an exemplary MBM proprotein according to FIGS. 1A-1C.

FIGS. 3A-3G are illustrations of embodiments of Type IIA MBM proproteins, in which in which an Fc region acts as a masking moiety by sterically hindering the TCE ABS from binding to its target. In the embodiments of FIG. 3A and FIG. 3B, the MBMs produced by the cleavage of Linker B are trivalent, with two TAA ABSs and one TCE ABS. In some embodiments, the trivalent MBMs are bispecific. In the embodiments of FIG. 3C through 3F, the MBM produced by the cleavage of Linker B are bivalent, with one TAA ABS and one TCE ABS. Although the TAA ABSs in FIGS. 3A-3F are illustrated as Fabs, the ABSs can be in other formats, e.g., scFvs or other formats described in Section 6.9. The TCE ABSs can be in the forms of Fabs (e.g., as illustrated in FIGS. 3A, 3C and 3E) or Fvs (e.g., as illustrated in FIGS. 3B, 3D and 3G). In all embodiments depicted in this figure, an MBM comprising the Fc domains is released following cleavage of Linker B.

FIG. 4 illustrates the mechanism of activation of an exemplary MBM proprotein according to FIGS. 3A-3G.

FIGS. 5A-5B depict an embodiment of a Type IB MBM proprotein, in which a TCE ABS is masked by an anti-TCE ABS. The MBM produced by the cleavage of Linker B is bivalent, with one TAA ABS and one TCE ABS, and lacks the Fc domains. Although the ABSs in FIG. 5A are illustrated as Fabs, the ABSs can be in other formats, e.g., scFvs or other formats described in Section 6.9.

FIG. 6 illustrates the mechanism of activation of an exemplary MBM proprotein according to FIGS. 5A-5B.

FIG. 7 is an illustration of an embodiment of a Type IIB MBM proprotein, in which an Fc region acts as a masking moiety by sterically hindering the TCE ABS from binding to its target. The MBM produced by the cleavage of Linker B is bivalent, with one TAA ABS and one TCE ABS, and lacks the Fc domains. Although the ABSs in FIG. 7 are illustrated as Fabs, the ABSs can be in other formats, e.g., scFvs or other formats described in Section 6.9.

FIG. 8 illustrates the mechanism of activation of an exemplary MBM proprotein according to FIG. 7.

FIG. 9 shows the result of a study showing the correlation between linker length and the ability of an Fc region to sterically hinder an anti-CD3 TCE ABS from binding to its target in constructs having the TCE ABS C-terminal to the Fc domains configured as shown in FIGS. 3A-3F and 7. For the purpose of demonstrating the effect of linker length, the linkers in this study did not include a substrate and were non-cleavable. FIG. 9 discloses SEQ ID NOs: 1 (1×G4S), 156 (3×G4S), and 153 (5×G4S).

FIG. 10 shows gel images displaying anti-TAA×CD3 antibodies run on non-reducing and reducing SDS-polyacrylamide gels after Protein A purification. Anti-TAA×CD3 bispecific antibodies tested had the configuration depicted in FIG. 7, where linkers A and B were either cleavable linkers as illustrated or replaced by noncleavable G4S (SEQ ID NO: 1) linkers.

FIG. 11 shows the results of a study demonstrating the inhibition of CD3 binding on JURKAT cells due to Fc steric hindrance in Type IIB MBM proprotein constructs configured as shown in FIG. 7. The curves show the inhibition of CD3 binding by Type IIB MBM proproteins relative to an anti-TAA×CD3 antibody in IgG format.

### 6. DETAILED DESCRIPTION

### 6.1. Definitions

As used herein, the following terms are intended to have the following meanings:

ABM proprotein: The term “ABM proprotein” (or “antigen-binding molecule proprotein”) as used herein refers to an ABM having reduced or abrogated ability to bind to a target recognized by at least one of its ABSs, e.g., due to the presence of a masking moiety. The masking moiety hinders binding of an ABS to its target and is separated from the ABM by proteolytic cleavage, e.g., by proteolytic cleavage of a protease cleavable linker connecting the masking moiety to the ABM.

ABS chain: Individual ABSs can exist as one (e.g., in the case of an scFv) polypeptide chain or form through the association of more than one polypeptide chains (e.g., in the case of a Fab). As used herein, the term “ABS chain” refers to all or a portion of an ABS that exists on a single polypeptide chain. The use of the term “ABS chain” is intended for convenience and descriptive purposes only and does not connote a particular configuration or method of production. Further, the reference to an ABS when describing an ABM, ABM proprotein, MBM or MBM proprotein encompasses an ABS chain unless the context dictates otherwise. Thus, when describing an ABM, ABM proprotein, MBM or MBM proprotein in which an Fc domain is operably linked to an ABS, the Fc domain may be covalently linked directly or indirectly (e.g., via a linker) through a peptide bond to, e.g., (1) a first ABS chain of a Fab (with the other components of the Fab on a second, associated ABS chain) or (2) the single ABS chain containing the scFv.

About, Approximately: The terms “about”, “approximately” and the like are used throughout the specification in front of a number to show that the number is not necessarily exact (e.g., to account for fractions, variations in measurement accuracy and/or precision, timing, etc.). It should be understood that a disclosure of “about X” or “approximately X” where X is a number is also a disclosure of “X.” Thus, for example, a disclosure of an embodiment in which one sequence has “about X % sequence identity” to another sequence is also a disclosure of an embodiment in which the sequence has “X % sequence identity” to the other sequence.

Activate, activation: The terms “activation”, “activation”, and the like in conjunction with an MBM proprotein of the disclosure refers to the protease-mediated enzymatic cleavage of a protease-cleavable linker that results in the unmasking of a TCE ABS and thus the production of an ABM or MBM with increased ability of the TCE ABS to bind to its target, for example through the release or separation of the TCE ABS from an anti-TCE ABS or an Fc domain that sterically hinders the binding of the TCE ABS to its target when the protease-cleavable linker is intact. Sometimes, activation is referred herein as “release” of the MBM, the masking moiety or the TCE ABS.

And, or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Anti-idiotype antibody, anti-idiotypic antibody: The terms “anti-idiotype antibody”, “anti-idiotypic antibody” and the like refer to an antibody that recognizes the idiotype of an antigen-binding site, e.g., an antigen-binding site specific for a TCR component such as CD3. The anti-idiotype antibody is capable of specifically binding to the variable region of the antigen-binding site and thereby reducing or preventing specific binding of the antigen-binding site to its cognate antigen. When associated with a molecule that comprises the antigen-binding site, the anti-idiotype antibody can function as a masking moiety of the molecule. The antigen-binding component of an anti-idiotypic antibody that recognizes the variable region of a T-cell engaging antibody, e.g., an antibody that recognizes CD3 or another component of the T-cell receptor, is often referred to herein as a “TCE ABS”.

Antibody: The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelized antibodies, chimeric antibodies, bispecific or multispecific antibodies and anti-idiotypic (anti-id) antibodies. The antibodies can be of any isotype/class (e.g., IgG, IgE, IgM, IgD, IgA and IgY) or subclass (e.g., IgG1, IgG2, IgG3, IgG4, IgA1 and IgA2). Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminus is a variable region and at the C-terminus is a constant region; the CH3 and CL domains represent the carboxy-terminus of the heavy and light chain, respectively, of natural antibodies. For convenience, and unless the context dictates otherwise, the reference to an antibody also refers to antibody fragments as well as engineered antibodies that include non-naturally occurring antigen-binding sites and/or antigen-binding sites having non-native configurations, e.g., a molecule that has a Fab or scFv domain C-terminal to the CH3 domain.

Antigen-binding molecule, ABM: The terms “antigen-binding molecule” and “ABM” as used herein refer to a molecule (e.g., an assembly of multiple polypeptide chains) comprising one or more antigen-binding sites. The ABMs of the disclosure can be monospecific or multispecific (e.g., bispecific). The antigen-binding sites in monospecific binding molecules all bind to the same epitope whereas multispecific binding molecules have at least two antigen-binding sites that bind to different epitopes, which can be one the same or different target molecules. In some embodiments, an antigen-binding molecule is an antibody.

Antigen-binding site: The term “antigen-binding site” or “ABS” as used herein refers to a portion of an antibody, ABM or MBM proprotein that has the ability to bind to an antigen non-covalently, reversibly and specifically in the absence of a masking. For example, in some embodiments, an ABS can be masked in the context of an ABM or MBM proprotein but has the ability to bind to an antigen non-covalently, reversibly and specifically when an ABM or MBM is produced by cleavage of a protease cleavable linker in the ABM or MBM proprotein. Examples of antigen-binding sites include antibody fragments such as, but not limited to, single-chain Fvs (scFv), Fab fragments, monovalent fragments consisting of the VL, VH, CL and CH1 domains; F(ab)2 fragments, bivalent fragments comprising two Fab fragments linked by a disulfide bridge at the hinge region; Fd fragments consisting of the VH and CH1 domains; Fv fragments consisting of the VL and VH domains of a single arm of an antibody; dAb fragments (Ward et al., 1989, Nature 341:544-546), which consist of a VH domain; VHH antibodies (also “VHH” or “Nanobody®”, Vincke et al., 2012, Methods Mol Biol. 911:15-26); and isolated complementarity determining regions (CDRs). Thus, the term “antibody fragment” encompasses both proteolytic fragments of antibodies (e.g., Fab and F(ab)₂ fragments) and engineered proteins comprising one or more portions of an antibody (e.g., an scFv). Antibody fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology 23:1126-1136).

Associated: The term “associated” in the context of an ABM or MBM proprotein refers to a functional relationship between two or more polypeptide chains. In particular, the term “associated” means that two or more polypeptides are associated with one another, e.g., non-covalently through molecular interactions or covalently through one or more disulfide bridges or chemical cross-linkages, so as to produce a functional MBM proprotein. Examples of associations that might be present in an MBM proprotein of the disclosure include (but are not limited to) associations between Fc regions in an Fc domain (homodimeric or, more preferably, heterodimeric as described in Section 6.10.2, associations between VH and VL regions in a Fab or Fv, and associations between CH1 and CL in a Fab.

Bivalent: The term “bivalent” as used herein in the context of an antigen-binding molecule refers to an antigen-binding molecule that has two antigen-binding sites. The domains can be the same or different. Accordingly, a bivalent antigen-binding molecule can be monospecific or bispecific.

Cancer: The term “cancer” refers to a disease characterized by the uncontrolled (and often rapid) growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, adrenal gland cancer, autonomic ganglial cancer, biliary tract cancer, bone cancer, endometrial cancer, eye cancer, fallopian tube cancer, genital tract cancers, large intestinal cancer, cancer of the meninges, oesophageal cancer, peritoneal cancer, pituitary cancer, penile cancer, placental cancer, pleura cancer, salivary gland cancer, small intestinal cancer, stomach cancer, testicular cancer, thymus cancer, thyroid cancer, upper aerodigestive cancers, urinary tract cancer, vaginal cancer, vulva cancer, lymphoma, leukemia, lung cancer and the like, e.g., any TAA-positive cancers of any of the foregoing types.

CD3: The term “CD3” refers to the cluster of differentiation 3 co-receptor (or co-receptor complex, or polypeptide chain of the co-receptor complex) of the T cell receptor. The amino acid sequence of the polypeptide chains of human CD3 are provided in NCBI Accession P04234, P07766 and P09693.

Complementarity Determining Region: The terms “complementarity determining region” or “CDR,” as used herein, refer to the sequences of amino acids within antibody variable regions which confer antigen specificity and binding affinity. For example, in general, there are three CDRs in each heavy chain variable region (e.g., CDR-H1, CDR-H2, and CDR-H3) and three CDRs in each light chain variable region (CDR-L1, CDR-L2, and CDR-L3). The precise amino acid sequence boundaries of a given CDR can be determined using any of a number of well-known schemes, including those described by Kabat et al., 1991, “Sequences of Proteins of Immunological Interest,” 5th Ed. Public Health Service, National Institutes of Health, Bethesda, MD (“Kabat” numbering scheme), Al-Lazikani et al., 1997, JMB 273:927-948 (“Chothia” numbering scheme) and ImmunoGen Tics (IMGT) numbering (Lefranc, 1999, The Immunologist 7:132-136; Lefranc et al., 2003, Dev. Comp. Immunol. 27:55-77 (“IMGT” numbering scheme). For example, for classic formats, under Kabat, the CDR amino acid residues in the heavy chain variable domain (VH) are numbered 31-35 (CDR-H1), 50-65 (CDR-H2), and 95-102 (CDR-H3); and the CDR amino acid residues in the light chain variable domain (VL) are numbered 24-34 (CDR-L1), 50-56 (CDR-L2), and 89-97 (CDR-L3). Under Chothia, the CDR amino acids in the VH are numbered 26-32 (CDR-H1), 52-56 (CDR-H2), and 95-102 (CDR-H3); and the amino acid residues in VL are numbered 26-32 (CDR-L1), 50-52 (CDR-L2), and 91-96 (CDR-L3). By combining the CDR definitions of both Kabat and Chothia, the CDRs consist of amino acid residues 26-35 (CDR-H1), 50-65 (CDR-H2), and 95-102 (CDR-H3) in human VH and amino acid residues 24-34 (CDR-L1), 50-56 (CDR-L2), and 89-97 (CDR-L3) in human VL. Under IMGT the CDR amino acid residues in the VH are numbered approximately 26-35 (CDR-H1), 51-57 (CDR-H2) and 93-102 (CDR-H3), and the CDR amino acid residues in the VL are numbered approximately 27-32 (CDR-L1), 50-52 (CDR-L2), and 89-97 (CDR-L3) (numbering according to “Kabat”). Under IMGT, the CDR regions of an antibody can be determined using the program IMGT/DomainGap Align.

Effector Function: The term “effector function” refers to an activity of an antibody molecule that is mediated by binding through a domain of the antibody other than the antigen-binding site, usually mediated by binding of effector molecules. Effector function includes complement-mediated effector function, which is mediated by, for example, binding of the C1 component of the complement to the antibody. Activation of complement is important in the opsonization and lysis of cell pathogens. The activation of complement also stimulates the inflammatory response and may also be involved in autoimmune hypersensitivity. Effector function also includes Fc receptor (FcR)-mediated effector function, which may be triggered upon binding of the constant domain of an antibody to an Fc receptor (FcR). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. An effector function of an antibody may be altered by altering, e.g., enhancing or reducing, the affinity of the antibody for an effector molecule such as an Fc receptor or a complement component. Binding affinity will generally be varied by modifying the effector molecule binding site, and in this case it is appropriate to locate the site of interest and modify at least part of the site in a suitable way. It is also envisaged that an alteration in the binding site on the antibody for the effector molecule need not alter significantly the overall binding affinity but may alter the geometry of the interaction rendering the effector mechanism ineffective as in non-productive binding. It is further envisaged that an effector function may also be altered by modifying a site not directly involved in effector molecule binding, but otherwise involved in performance of the effector function.

Epitope: An epitope, or antigenic determinant, is a portion of an antigen recognized by an antibody or other antigen-binding moiety as described herein. An epitope can be linear or conformational.

Fab: The term “Fab” refers to a pair of polypeptide chains, the first comprising a variable heavy (VH) domain of an antibody operably linked (typically N-terminal to) to a first constant domain (referred to herein as C1), and the second comprising variable light (VL) domain of an antibody N-terminal operably linked (typically N-terminal) to a second constant domain (referred to herein as C2) capable of pairing with the first constant domain. In a native antibody, the VH is N-terminal to the first constant domain (CH1) of the heavy chain and the VL is N-terminal to the constant domain of the light chain (CL). The Fabs of the disclosure can be arranged according to the native orientation or include domain substitutions or swaps that facilitate correct VH and VL pairings. For example, it is possible to replace the CH1 and CL domain pair in a Fab with a CH3-domain pair to facilitate correct modified Fab-chain pairing in heterodimeric molecules. It is also possible to reverse CH1 and CL, so that the CH1 is attached to VL and CL is attached to the VH, a configuration generally known as Crossmab. The term “Fab” encompasses single chain Fabs.

Fc Domain and Fc Region: The term “Fc domain” refers to a portion of the heavy chain that pairs with the corresponding portion of another heavy chain. The term “Fc region” refers to the region of antibody-based binding molecules formed by association of two heavy chain Fc domains. The two Fc domains within the Fc region may be the same or different from one another. In a native antibody the Fc domains are typically identical, but one or both Fc domains might advantageously be modified to allow for heterodimerization, e.g., via a knob-in-hole interaction.

Fv: The term “Fv” refers to the minimum antibody fragment derivable from an immunoglobulin that contains a complete target recognition and binding site. This region consists of a dimer of one heavy and one light chain variable domain in a tight, noncovalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target. The reference to a VH-VL dimer herein is not intended to convey any particular configuration. By way of example and not limitation, the VH and VL can come together in any configuration described herein to form a half antibody, or can each be present on a separate half antibody and come together to form an antigen binding domain when the separate half antibodies associate, for example to form a ABM or MBM proprotein of the disclosure. When present on a single polypeptide chain (e.g., a scFv), the VH and be N-terminal or C-terminal to the VL.

Half Antibody: The term “half antibody” refers to a molecule that comprises at least one ABS or ABS chain and can associate with another molecule comprising an ABS or ABS chain through, e.g., a disulfide bridge or molecular interactions (e.g., knob-in-hole interactions between Fc heterodimers). A half antibody can be composed of one polypeptide chain or more than one polypeptide chains (e.g., the two polypeptide chains of a Fab). In a preferred embodiment, a half-antibody comprises an Fc region. An example of a half antibody is a molecule comprising a heavy and light chain of an antibody (e.g., an IgG antibody). Another example of a half antibody is a molecule comprising a first polypeptide comprising a VL domain and a CL domain, and a second polypeptide comprising a VH domain, a CH1 domain, a hinge domain, a CH2 domain, and a CH3 domain, wherein said VL and VH domains form an ABS. Yet another example of a half antibody is a polypeptide comprising an scFv domain, a CH2 domain and a CH3 domain.

A half antibody might include more than one ABS, for example a half-antibody comprising (in N-to C-terminal order) an VH1 domain, a CH1 domain, a CH2 domain, a CH3 domain, and another VH1 domain or an scFv domain. Half antibodies might also include an ABS chain that when associated with another ABS chain in another half antibody forms a complete ABS.

Thus, an ABM or MBM proprotein can comprise one, more typically two, or even more than two half antibodies, and a half antibody can comprise one or more ABSs or ABS chains.

In some ABM or MBM proproteins, a first half antibody will associate, e.g., heterodimerize, with a second half antibody. In other ABM or MBM proproteins, a first half antibody will be covalently linked to a second half antibody, for example through disulfide bridges or chemical crosslinking. In yet other ABM or MBM proproteins, a first half antibody will associate with a second half antibody through both covalent attachments and non-covalent interactions, for example disulfide bridges and knob-in-hole interactions.

The term “half antibody” is intended for descriptive purposes only and does not connote a particular configuration or method of production. Descriptions of a half antibody as a “first” half antibody, a “second” half antibody, a “left” half antibody, a “right” half antibody or the like are merely for convenience and descriptive purposes.

Host cell or recombinant host cell: The terms “host cell” or “recombinant host cell” refer to a cell that has been genetically-engineered, e.g., through introduction of a heterologous nucleic acid. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A host cell may carry the heterologous nucleic acid transiently, e.g., on an extrachromosomal heterologous expression vector, or stably, e.g., through integration of the heterologous nucleic acid into the host cell genome. For purposes of expressing an ABM or MBM proprotein of the disclosure, a host cell is preferably a cell line of mammalian origin or mammalian-like characteristics, such as monkey kidney cells (COS, e.g., COS-1, COS-7), HEK293), baby hamster kidney (BHK, e.g., BHK21), Chinese hamster ovary (CHO), NSO, PerC6, BSC-1, human hepatocellular carcinoma cells (e.g., Hep G2), SP2/0, HeLa, Madin-Darby bovine kidney (MDBK), myeloma and lymphoma cells, or derivatives and/or engineered variants thereof. The engineered variants include, e.g., derivatives that grow at higher density than the original cell lines and/or glycan profile modified derivatives and and/or site-specific integration site derivatives.

Linker: The term “linker” as used herein refers to a protease-cleavable linker or a non-cleavable linker.

Masking Moiety: The term “masking moiety” as used herein in relation to an MBM proprotein refers an amino acid sequence in an MBM proprotein that inhibits a TCE ABS's ability to specifically bind its target, either through a specific interaction with the TCE ABS (e.g., where the masking moiety is an anti-idiotype of the TCE ABS or “anti-TCE ABS”) or through positioning of the TCE ABS relative to another component of the MBM proprotein that sterically hinders the binding of the TCE ABS to its target (e.g., by connecting the TCE ABS to the Fc region through short linkers). The masking moiety and the TCE ABS are arranged in the MBM proprotein such that cleavage of a protease cleavable linker reduces the inhibition of the TCE ABS's interaction with its target, either through the generation of an MBM that lacks the masking moiety or an MBM in which spatial constrains on the TCE ABS's ability to interact with its target are alleviated. The term “masking moiety” when used in relation to an antigen binding-molecule more generally refers to an amino acid sequence in the antigen-binding molecule proprotein that inhibits the ability of an antigen binding site (ABS) in the antigen-binding molecule to specifically bind its target, either through a specific interaction with the ABS (e.g., where the masking moiety is an anti-idiotype of the ABS) or through positioning of the ABS relative to another component of the antigen-binding molecule that sterically hinders the binding of the ABS to its target (e.g., by connecting the ABS to an Fc region through short linkers). Generally, the masking moiety and the ABS are arranged in the antigen-binding molecule such that cleavage of a protease cleavable linker reduces the inhibition of the ABS's interaction with its target, either through the generation of an antigen-binding molecule that lacks the masking moiety or an antigen-binding molecule in which spatial constrains on the ABS's ability to interact with its target are alleviated.

MBM proprotein: The term “MBM proprotein” as used herein refers to an MBM having reduced or abrogated ability to bind to a target recognized by at least one of its ABSs, e.g., due to the presence of a masking moiety. The masking moiety hinders binding of an ABS to its target is separated from the MBM by proteolytic cleavage, e.g., by proteolytic cleavage of a protease cleavable linker connecting the masking moiety to the MBM. Typically, in the MBM proproteins of the disclosure, the TCE ABS is masked while a TAA ABS is not. The TAA ABS can serve as a targeting moiety to direct the MBM proprotein to the site of a tumor, wherein in the presence of proteases that recognize a substrate in the protease cleavable linker results in cleavage of the linker, restoring the binding of the TCE ABS to its target and the ability of the MBM to activate the T-cell against tumor cells that express the TAA.

Monovalent: The term “monovalent” as used herein in the context of an antigen-binding molecule refers to an antigen-binding molecule that has a single antigen-binding site.

Multispecific binding molecule, MBM: The terms “multispecific binding molecule” and “MBM” refer to a molecule that comprises two or more antigen binding sites. Typically, the MBMs comprise a TAA ABS and a TCE ABS.

Non-cleavable linker: A non-cleavable linker refers to a peptide whose amino acid sequence lacks a substrate sequence for a protease, e.g., a protease as described in Section 6.4.1, that recognizes and cleaves a specific sequence motif, e.g., a substrate as described in Section 6.4.2.

Operably linked: The term “operably linked” refers to a functional relationship between two or more peptide or polypeptide domains or nucleic acid (e.g., DNA) segments. In the context of a fusion protein or other polypeptide, the term “operably linked” means that two or more amino acid segments are linked so as to produce a functional polypeptide. For example, in the context of an ABM or MBM proprotein of the disclosure, separate ABSs (or chains of an ABS) can be operably linked through peptide linker sequences. In the context of a nucleic acid encoding a fusion protein, such as a polypeptide chain of an ABM or MBM of the disclosure, “operably linked” means that the two nucleic acids are joined such that the amino acid sequences encoded by the two nucleic acids remain in-frame. In the context of transcriptional regulation, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system.

Polypeptide, Peptide and Protein: The terms “polypeptide”, “peptide” and “protein” are used interchangeably herein to refer to a polymer of amino acid residues.

Proprotein: A “proprotein” is a protein precursor that is inactive and which can be activated by proteolysis by a protease. Thus, proproteins are “protease activatable”.

Protease: The term “protease” as used herein refers to any enzyme that that catalyzes hydrolysis of a peptide bond. Generally, the proteases useful in the present disclosure, e.g., the proteases described in Section 6.4.1, recognize and cleaves a specific sequence motif, e.g., a substrate as described in Section 6.4.2. Preferably, the proteases are expressed at higher levels in cancer tissues as compared to normal tissues.

Protease cleavable linker: As used herein, the term “protease cleavable linker” or “PCL” refers to a peptide whose amino acid sequence contains one or more (e.g., two or three) substrate sequences for one or more proteases.

Recognize: The term “recognize” as used herein refers to an ABS that finds and interacts (e.g., binds) with its epitope.

Single Chain Fab or scFab: The term “single chain Fab” or “scFab” as used herein refers an ABS comprising a VH domain, a CH1 domain, a VL domain, a CL domain and a linker. In some embodiments, the foregoing domains and linker are arranged in one of the following orders in a N-terminal to C-terminal orientation: (a) VH-CH1-linker-VL-CL, (b) VL-CL-linker-VH-CH1, (c) VH-CL-linker-VL-CH1 or (d) VL-CH1-linker-VH-CL. Linkers are suitably noncleavable linkers of at least 30 amino acids, preferably between 32 and 50 amino acids. Single chain Fab fragments are typically stabilized via the natural disulfide bond between the CL domain and the CH1 domain. In addition, these single chain Fab molecules might be further stabilized by generation of interchain disulfide bonds via insertion of cysteine residues (e.g., at position 44 in the VH domain and position 100 in the VL domain according to Kabat numbering).

Single Chain Fv or scFv: The term “single-chain Fv” or “scFv” as used herein refers to ABSs comprising the VH and VL domains of an antibody, wherein these domains are present in a single polypeptide chain. Preferably, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds. (1994), Springer-Verlag, New York, pp. 269-315. The VH and VL and be arranged in the N- to C-terminal order VH-VL or VL-VH, typically separated by a linkers, for example a linker as set forth in Table E.

Spacer: As used herein, the term “spacer” refers to a peptide, the amino acid sequence of which is not a substrate for a protease, incorporated into a linker containing a substrate. A spacer can be used to separate the substrate from other domains in a molecule, for example an ABS. In some aspects, residues in the spacer minimize aminopeptidase and/or exopeptidase action to prevent cleavage of N-terminal amino acids.

Specifically (or selectively) binds: The term “specifically (or selectively) binds” to an antigen or an epitope refers to a binding reaction that is determinative of the presence of a cognate antigen or an epitope in a heterogeneous population of proteins and other molecules. The binding reaction can be but need not be mediated by an antibody or antibody fragment. The term “specifically binds” does not exclude cross-species reactivity. For example, an antigen-binding site (e.g., an antigen-binding fragment of an antibody) that “specifically binds” to an antigen from one species may also “specifically bind” to that antigen in one or more other species. Thus, such cross-species reactivity does not itself alter the classification of an antigen-binding site as a “specific” binder. In certain embodiments, an antigen-binding site of the disclosure (e.g., a “TAA ABS”) that specifically binds to a human antigen has cross-species reactivity with one or more non-human mammalian species, e.g., a primate species (including but not limited to one or more of Macaca fascicularis, Macaca mulatta, and Macaca nemestrina) or a rodent species, e.g., Mus musculus.

Subject: The term “subject” includes human and non-human animals. Non-human animals include all vertebrates, e.g., mammals and non-mammals, such as non-human primates, sheep, dog, cow, chickens, amphibians, and reptiles. In preferred embodiments, the subject is human.

Substrate: The term “substrate” refers to peptide sequence on which a protease will act and within which the protease will cleave a peptide bond.

TAA ABS: The term “TAA ABS” refers to an ABS that recognizes or specifically binds to an extracellular matrix (“ECM”) protein, a tumor reactive lymphocyte antigen, a cell surface molecule of tumor or viral lymphocytes, a T-cell antigen (“TCA”), a checkpoint inhibitor, or a TAA. The skilled artisan would recognize that the foregoing categories of target molecules are not mutually exclusive and thus a given target molecule may fall into more than one of the foregoing categories of target molecules. For example, some molecules may be considered both TAAs and ECM proteins, and other molecules may be considered both TCAs and checkpoint inhibitors.

T-Cell Antigen, TCA: The term “T-cell antigen” or “TCA” refers to a molecule (typically a protein, carbohydrate, lipid or some combination thereof) that is expressed on the surface of a T-lymphocyte and is useful for the preferential targeting of a pharmacological agent to a particular site. In some embodiments, the site is cancer tissue and/or the T-cell antigen is a tumor reactive lymphocyte antigen, a cell surface molecule of tumor or viral lymphocytes, or a checkpoint inhibitor expressed on a T-lymphocyte.

TCE ABS: The term “TCE ABS” refers to an ABS that recognizes or specifically binds to a T-cell antigen in a manner that can initiate signaling through the TCR complex. Exemplary T-cell antigens recognized or specifically bound by a TCE ABS are CD3 and other components of the TCR complex.

T-cell Receptor, TCR: The “T-cell receptor” or “TCR” is the component of the T-cell that is responsible for interacting with and sensing the targets of T-cell adaptive immunity. In general terms, the TCR is comprised of a heterodimeric protein complex presented on the cell surface. T cells may be broadly classified as αβ or γδ according to the somatically rearranged TCR form they express at their surface. There exist two TCR chain pair forms: TCRα and TCRβ pairs TCRγ and TCRδ. Mature αβ and γδ TCR chain pairs are presented at the cell surface in a complex with a number of accessory CD3 subunits, denoted ε, γ, δ and ζ. These subunits associate with αβ or γδ TCRs as three dimers (εγ, εδ, ζζ). This TCR complex forms the unit for initiation of cellular signaling responses upon engagement of a TCR αβ or TCRγδ with cognate antigen. The terms “T-cell receptor complex” and “TCR complex” refer to complexes of TCR αβ or TCRγδ and CD3.

Trivalent: The term “trivalent” as used herein in the context of an antigen-binding molecule refers to an antigen-binding molecule that has three antigen-binding sites. The antigen-binding sites can all be the same, all be different, or include two of the same antigen-binding site and a third, different antigen-binding site.

Tumor: The term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

Tumor-Associated Antigen: The term “tumor-associated antigen” or “TAA” refers to a molecule (typically a protein, carbohydrate, lipid or some combination thereof) that is expressed on the surface of a cancer cell, either entirely or as a fragment (e.g., MHC/peptide), and which is useful for the preferential targeting of a pharmacological agent to the cancer cell. In some embodiments, a TAA is a marker expressed by both normal cells and cancer cells, e.g., a lineage marker. In some embodiments, a TAA is a cell surface molecule that is overexpressed in a cancer cell in comparison to a normal cell, for instance, 1-fold over expression, 2-fold overexpression, 3-fold overexpression or more in comparison to a normal cell. In some embodiments, a TAA is a cell surface molecule that is inappropriately synthesized in the cancer cell, for instance, a molecule that contains deletions, additions or mutations in comparison to the molecule expressed on a normal cell. In some embodiments, a TAA will be expressed exclusively on the cell surface of a cancer cell, entirely or as a fragment (e.g., MHC/peptide), and not synthesized or expressed on the surface of a normal cell. Accordingly, the term “TAA” encompasses antigens that are specific to cancer cells, sometimes known in the art as tumor-specific antigens (“TSAs”).

TAA ABS: The term “TAA ABS” as used herein refers to an antigen-binding site that recognizes or specifically binds a TAA.

Treat, Treatment, Treating: As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more ABM or MBM proproteins of the disclosure. In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

Universal Light Chain, UCL: The term “universal light chain” or “ULC” as used herein refers to a light chain variable region (VL) that can pair with more than on heavy chain variable region (VL). In the context of an ABS, the term “universal light chain” or “ULC” refers to a light chain polypeptide capable of pairing with the heavy chain region of the ABS and also capable of pairing with other heavy chain regions. ULCs can also include constant domains, e.g., a CL domain of an antibody. Universal light chains are also known as “common light chains.

VH: The term “VH” refers to the variable region of an immunoglobulin heavy chain of an antibody, including the heavy chain of an Fv, scFv, dsFv or Fab.

VL: The term “VL” refers to the variable region of an immunoglobulin light chain, including the light chain of an Fv, scFv, dsFv or Fab.

### 6.2. Antigen-Binding Molecule Proproteins

The present disclosure relates to antigen-binding molecule proproteins comprising an ABS, a masking moiety, and a protease-cleavable linker (PCL), arranged so that the masking moiety diminishes or blocks the ABS from binding to its target and configured such that upon encountering a protease, e.g., a protease that is overexpressed in the tumor environment, the masking moiety is cleaved and a binding molecule is produced having enhanced target binding. Typically, the masking moiety of an antigen-binding molecule proprotein masks the ABS via steric hindrance (e.g., an Fc domain masking moiety) and/or via binding of a targeting moiety to the ABS (e.g., an anti-ABS binding moiety).

Accordingly, the antigen-binding molecule proproteins of the disclosure generally comprise: (a) a masking moiety, and (b) a an ABS or component thereof connected to the masking moiety via a PCL, where the ABS is hindered (e.g., sterically hindered) from binding its target. Exemplary masking moieties include Fc domains and anti-ABS binding moieties. Use of an Fc domain as a masking moiety provides for steric hindrance of the ABS, which steric hindrance is released following protease cleavage of the PCL (e.g., by a tumor-specific protease). Particular example Fc domain-masked antigen-binding molecule proproteins include Type IIA and Type IIB MBM proproteins (e.g., as described in Section 6.3.2). Use of an anti-ABS binding moiety as a masking moiety blocks binding of the ABS to its target, which blockage is released following protease cleavage of the PCL (e.g., by a tumor-specific protease). Particular example anti-ABS binding moiety-masked antigen-binding molecule proproteins include Type IA and Type IB MBM proproteins (e.g., as described in Section 6.3.1.1). An antigen-binding molecule proprotein may comprise multiple masking moieties, for example an Fc domain and also an anti-ABS binding moiety, where one or more of the masking moieties are released following protease cleavage of the PCL.

The PCL comprises one or more substrates that are recognized and cleaved by one or more proteases, e.g., one or more of the proteases described in Section 6.4.1. Exemplary substrate sequences are described in Section 6.4.2. The PCL may further include spacer sequences, e.g., as described in Section 6.4.3. Examples of entire protease-cleavable linkers comprising substrate and spacer sequences are provided in Section 6.4.4.

Without being bound by theory, it is believed that a PCL connecting an ABS and a masking moiety (particularly a masking moiety that sterically hinders binding of the ABS to its target such as an Fc domain) being of small length (e.g., 30 or fewer) provides for maximal hindrance of the ABS binding prior to protease cleavage of the PCL, thereby minimizing off-target effects of the ABS. Accordingly, in certain aspects, the PCL is 30 or fewer, 29 or fewer, 28 or fewer, 27 or fewer, 26 or fewer, 25 or fewer, 24 or fewer, 23 or fewer, 22 or fewer, 21 or fewer, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, or 8 or fewer amino acids in length.

Antigen-binding molecule proproteins generally comprise at least two polypeptide chains, where a first polypeptide chain comprises the masking moiety, the PCL, and a first component of the ABS (e.g., a VH), and a second polypeptide chain comprises a second component of the ABS that pairs with the first component to form the ABS (e.g., a VL). In cases where the masking moiety is an Fc domain, the second polypeptide may further comprise an additional Fc domain that pairs with the first Fc domain to form an Fc region (e.g., an Fc heterodimer). The ABS may be, for example, a TAA ABS (e.g., as described in Section 6.6) or a TCE ABS (e.g., as described in Section 6.7).

In some aspects, the antigen-binding molecule proprotein further comprises an additional ABS which is not masked by the masking moiety. In such cases, the first ABS may be a TCE ABS while the additional ABS is a TAA ABS; alternatively the first ABS may be a TAA ABS while the additional ABS is a TCE ABS. An antigen-binding molecule proprotein comprising two or more ABSs may, upon protease cleavage of the PCL, release a multispecific binding molecule (e.g., bispecific or trispecific binding molecule). Certain examples of antigen-binding molecule proproteins having multiple ABSs are described in Section 6.3, infra.

### 6.3. MBM Proproteins

In some embodiments, the present disclosure relates to MBM proproteins comprising a TCE ABS, a TAA ABS, a masking moiety, and a protease-cleavable linker, arranged so that the masking moiety diminishes or blocks the TCE ABS from binding to its target. The MBM proprotein is configured such that upon encountering a protease, e.g., a protease that is overexpressed in the tumor environment, the masking moiety is cleaved and an MBM is produced in which the inhibition of TCE ABS binding is reversed. Typically, the MBM proproteins of the disclosure further comprise two Fc domains that associate for form an Fc region, and the TCE ABS is C-terminal to the Fc region.

Accordingly, the MBM proproteins of the disclosure generally comprise: (a) a first Fc domain and a second Fc domain capable of associating to form an Fc region; (b) a TCE ABS C-terminal to the first Fc domain and/or the second Fc domain; (c) a TAA ABS; and (d) a protease-cleavable linker (PCL) C-terminal to the first Fc domain and/or the second Fc domain. Typically, the TAA ABS in the MBM proprotein is capable of binding to its target whether or not the protease-cleavable linker is in the uncleaved state while the binding of the TCE ABS to its target is significantly enhanced following protease cleavage of the protease-cleavable linker. Following cleavage of the protease-cleavable linker, e.g., in the tumor environment, an MBM is released comprising the TAA ABS and the TCE ABS.

In Type I MBM proproteins, the TCE ABS is masked by an anti-idiotypic antibody. In Type II MBM proproteins, the Fc region acts as the masking moiety by sterically hindering the TCE ABS from binding to its target.

The protease-cleavable linkers in Type IA, Type IIA, Type IB and Type II MBM proproteins comprise one or more substrates that are recognized and cleaved by one or more proteases, e.g., one or more of the proteases described in Section 6.4.1. Exemplary substrate sequences are described in Section 6.4.2. The protease-cleavable linkers typically further include spacer sequences, e.g., as described in Section 6.4.3. Examples of entire protease-cleavable linkers comprising substrate and spacer sequences are provided in Section 6.4.4. Depending on the configuration of the MBM proprotein, the Fc region may be retained in the resulting MBM following cleavage of the protease-cleavable linker, e.g., following cleavage of the protease-cleavable linker in the Type IA and Type IIA MBM proproteins, or may be released from the resulting MBM following cleavage of the protease-cleavable linker, e.g., following cleavage of the protease-cleavable linker in Type IB and Type IIB MBM proproteins.

Generally, the MBM proproteins of the disclosure contain multiple linkers. Preferably, other than the protease-cleavable linker whose cleavage releases the MBM, the remaining linkers are non-cleavable. Examples of non-cleavable linkers are set forth in Section 6.5.

Exemplary TAA ABSs that can be used in the MBM proproteins of the disclosure (e.g., Type IA, Type IIA, Type IB and Type II MBM proproteins of the disclosure) are disclosed in Section 6.6. Exemplary TCE ABSs that can be used in the MBM proproteins of the disclosure (e.g., Type IA, Type IIA, Type IB and Type II MBM proproteins of the disclosure) are disclosed in Section 6.7. Exemplary anti-TCE ABSs that can be used in the Type I MBM proproteins of the disclosure are disclosed Section 6.8. Section 6.9 describes suitable formats for the ABSs that are incorporated in the MBM proproteins of the disclosure. Section 6.10 describes suitable Fc domains that can be incorporated in the MBM proproteins of the disclosure (and which can be used as masking moieties in the Type II MBM proproteins of the disclosure).

6.3.1. Type I MBM Proproteins (with Anti-Idiotypic Masking Moieties)

The present disclosure provides MBM proproteins comprising a TCE ABS masked by an anti-TCE ABS, referred to herein as “Type I” MBM proproteins. Typically, both the TCE ABS and the anti-TCE ABS are arranged C-terminal to an Fc region, with each operably linked to a separate Fc domain. The TCE ABS or the anti-TCE ABS can be separated from one of the Fc domains of the Fc region by a protease-cleavable linker, e.g., as described in Section 6.4. In the Type IA MBM proproteins, the Fc region is retained in the MBM activated by cleavage of the protease-cleavable linker. In the Type IB MBM proproteins, the Fc region is separated from the MBM activated by cleavage of the protease-cleavable linker.

### 6.3.1.1. Type IA MBM Proproteins

Generally, Type IA MBM proproteins comprise first polypeptide chain comprising, in an N-terminal to C-terminal orientation, an Fc domain (with an optional hinge domain at its N-terminus), a non-cleavable linker, and a TCE ABS (or TCE ABS chain), and a second polypeptide chain comprising, in an N-terminal to C-terminal orientation, an Fc domain (with an optional hinge domain at its N-terminus), a protease-cleavable linker, and an anti-TCE ABS (or anti-TCE ABS chain). One or both Fc domains preferably further comprise a TAA ABS (or TAA ABS chain) at its N-terminus. When the TCE ABS, TAA ABS and/or anti-TCE ABS are composed of multiple polypeptide chains, the MBM proprotein will further comprise the other chain(s), for example the light chain of a Fab.

Following cleavage of the protease-cleavable linker the anti-TCE ABS is released, producing an MBM comprising first polypeptide chain comprising, in an N-terminal to C-terminal orientation, an Fc domain (with an optional hinge domain at its N-terminus), a non-cleavable linker, and a TCE ABS, and a second polypeptide chain comprising, in an N-terminal to C-terminal orientation, an Fc domain (with an optional hinge domain at its N-terminus), and a portion of the protease-cleavable linker. One or both Fc domains preferably further comprise a TAA ABS at its N-terminus. When the TCE ABS and/or TAA ABS are composed of multiple polypeptide chains, the resulting MBM will further comprise the other chain(s), for example the light chain of a Fab.

Preferably, the resulting MBM is bispecific for the TAA and the TCE ABS's target (e.g., CD3).

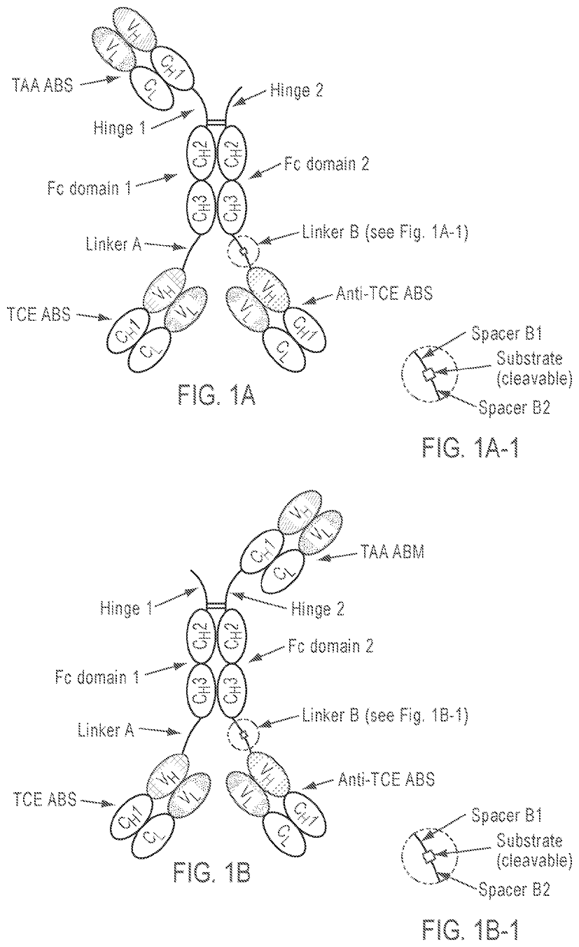

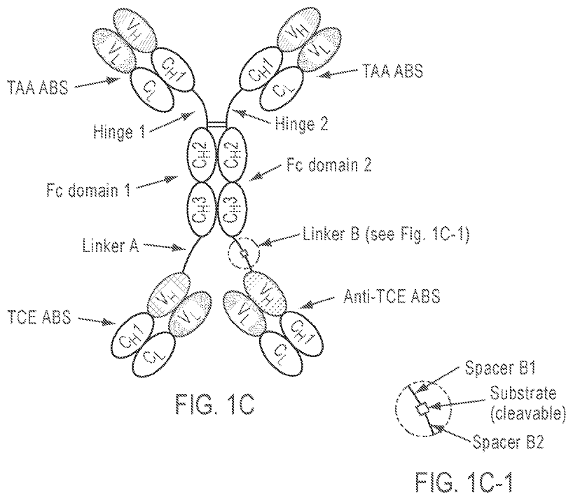

FIGS. 1A to 1C illustrate exemplary embodiments of Type IA MBM proproteins. As shown in FIGS. 1A and 1B, the Type IA MBM proproteins can include only a single TAA ABS. Alternatively, as shown in FIG. 1C, Type IA MBM proproteins can include two TAA ABSs. Although depicted as a Fab for illustrative purposes, the Anti-TCE ABS of FIGS. 1A to 1C may be an scFv, a VHH, a VH, or other fragment or region from an anti-idiotypic antibody. The depiction of a single substrate and two spacers in the protease-cleavable linker of FIGS. 1A-1C is for illustrative purposes only; as described in Section 6.4, protease-cleavable linkers can include multiple substrates and more than two spacer sequences.

### 6.3.1.2. Type IB MBM Proproteins

Generally, Type IB MBM proproteins comprise first polypeptide chain comprising, in an N-terminal to C-terminal orientation, an Fc domain (with an optional hinge domain at its N-terminus), a non-cleavable linker, and an anti-TCE ABS (or anti-TCE ABS chain), and a second polypeptide chain comprising, in an N-terminal to C-terminal orientation, an Fc domain (with an optional hinge domain at its N-terminus), a protease-cleavable linker, a TCE ABS (or TCE ABS chain) and a TAA ABS (or TAA ABS chain), generally with the TCE ABS and the TAA ABS separated by a non-cleavable linker. When the TCE ABS, TAA ABS and/or anti-TCE ABS are composed of multiple polypeptide chains, the MBM proprotein will further comprise the other chain(s), for example the light chain of a Fab.

Following cleavage of the protease-cleavable linker an MBM is released comprising the TCE ABS, optional linker and the TAA ABS. When the TCE ABS and/or TAA ABS are composed of multiple polypeptide chains, the resulting MBM will further comprise the other chain(s), for example the light chain of a Fab.

Preferably, the resulting MBM is bispecific for the TAA and the TCE ABS's target (e.g., CD3).

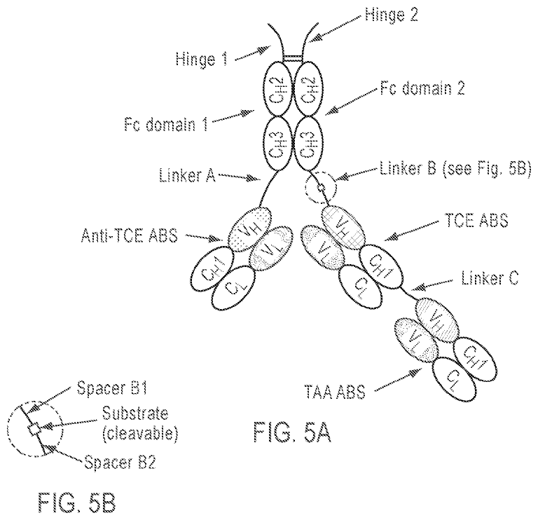

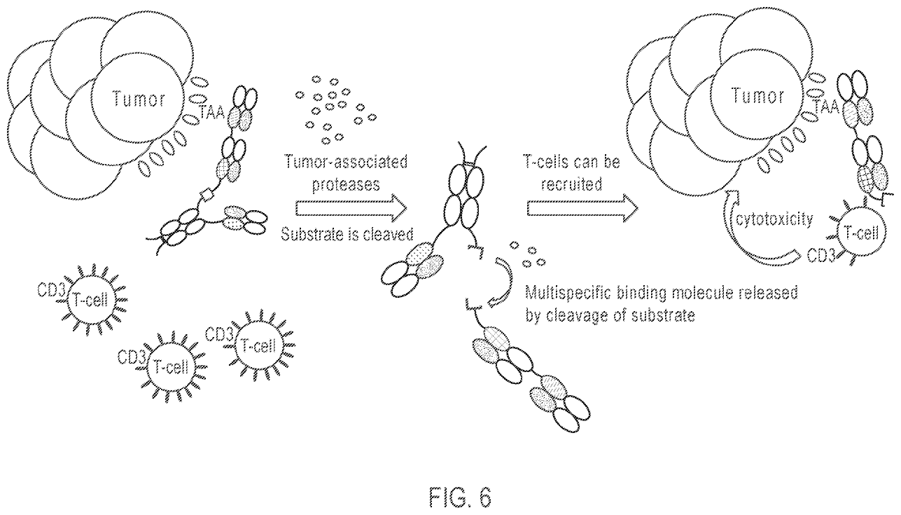

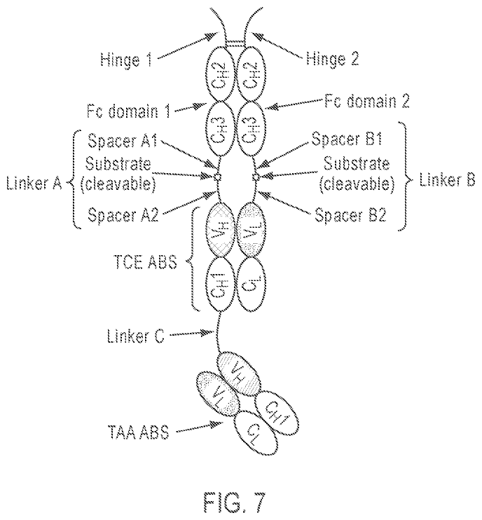

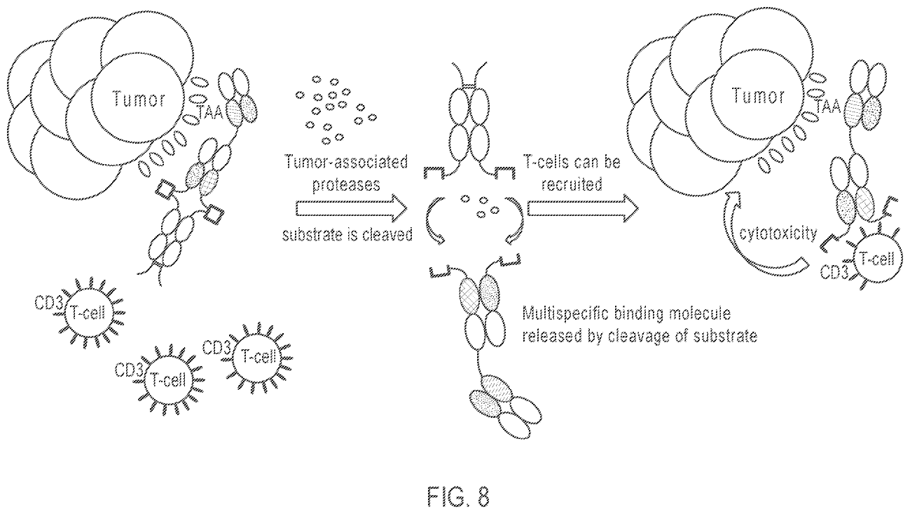

FIGS. 5A-5B illustrate an exemplary embodiment of a Type IB MBM proprotein. Although depicted as a Fab for illustrative purposes, the Anti-TCE ABS of FIG. 5A may be an scFv, a VHH, a VH, or other fragment or region from an anti-idiotypic antibody. The depiction of a single substrate and two spacers in the protease-cleavable linker of FIGS. 5A-5B is for illustrative purposes only; as described in Section 6.4, protease-cleavable linkers can include multiple substrates and more than two spacer sequences.

6.3.2. Type II MBM proproteins (with sterically hindering masking moieties)

### 6.3.2.1. Type IIA MBM Proproteins

Generally, Type IIA MBM proproteins comprise first polypeptide chain comprising, in an N-terminal to C-terminal orientation, an Fc domain (with an optional hinge domain at its N-terminus), a non-cleavable linker, and a first TCE ABS chain, and a second polypeptide chain comprising, in an N-terminal to C-terminal orientation, an Fc domain (with an optional hinge domain at its N-terminus), a protease-cleavable linker, and the second TCE ABS chain. The TCE ABS chains may include a VH and a VL without corresponding CH1 and CL domain domains, such that a TCE ABS in the form of an Fv is formed by the association of the TCE ABS chains. The TCE ABS chains may include a VH and a VL with corresponding CH1 and CL domain domains, such that a TCE ABS in the form of a Fab is formed by the association of the TCE ABS chains. In some embodiments, the VH and/or VL of the TCE ABS chains attached to the Fc domains comprise a deletion of one or more amino acids at or near their N-terminus, e.g., an N-terminal truncation. A VH or VL may have such a deletion precisely at the N-terminus (i.e., may be a truncation), or in some cases within 1, 2, 3, 4, 5, 6, 7, 8, 9, or 10 amino acids of the N-terminus. In some embodiments, the VH of a TCE ABS of a Type IIA MBM proprotein of the present disclosure comprises a deletion (e.g., a truncation) or 1, 2, 3, 4, 5, 6, 7, 8, 9, or 10 amino acids at or near (e.g., within 10 amino acids of) the N-terminus of a reference VH, e.g., a wild-type VH or the VH upon which the TCE ABS is based, such as the VH of an antibody or antibody sequence of Table G. In some embodiments, the VL of a TCE ABS of a Type IIA MBM proprotein of the present disclosure comprises a deletion (e.g., a truncation) of 1, 2, 3, 4, 5, 6, 7, 8, 9, or 10 amino acids at or near (e.g., within 10 amino acids of) the N-terminus of a reference VL, e.g., a wild-type VL or the VL upon which the TCE ABS is based, such as the VL of an antibody or antibody sequence of Table G.

The length of the linkers separating the Fc domains and the TCE ABS chains is chosen so as to optimize the ability of the Fc domains to sterically hinder the TCE ABS from binding its target. Preferably, the length of non-cleavable linker typically ranges between 5 and 30 amino acids, and in various embodiments is 25 amino acids or less (e.g., ranges between 5 and 25 amino acids), 20 amino acids or less (e.g., ranges between 5 and 20 amino acids), 15 amino acids or less (e.g., ranges between 5 and 15 amino acids), or 10 amino acids or less (e.g., ranges between 5 and 10 amino acids). In particular embodiments, the non-cleavable linker is 5, 6, 7, 8, 9 or 10 amino acids in length.

One or both Fc domains preferably further comprise a TAA ABS (or TAA ABS chain) at its N-terminus. When the TAA ABS is composed of multiple polypeptide chains, the MBM proprotein will further comprise the other chain, for example the light chain of a Fab.

One of both Fc domains may, in some embodiments, comprise a deletion of one or more amino acids at or near the C-terminus (e.g., a C-terminal truncation) relative to a wild-type Fc domain. In some embodiments, one or both Fc domains of a Type IIA MBM proprotein of the present disclosure comprise a deletion (e.g., a truncation) of 1, 2, 3, 4, 5, 6, 7, 8, 9, or 10 amino acids at or near the C-terminus relative to a wild-type Fc domain. An Fc domain may have such a deletion precisely at the C-terminus (i.e., may be a truncation), or in some cases within 1, 2, 3, 4, 5, 6, 7, 8, 9, or 10 amino acids of the C-terminus.

In particular embodiments, a Type IIA MBM proprotein of the disclosure comprises (1) Fc domains having a deletion (e.g., a truncation) of 1, 2, 3, 4, 5, or 6 amino acids at or near the C-terminus relative to a wild-type Fc domain; and (2) TCE ABS chains having VH and VL domains with a deletion (e.g., a truncation) of 1, 2, 3, 4, 5, or 6 amino acids at or near the N-terminus. For example, a Type IIA MBM proprotein may comprise (1) two Fc domains, each having a deletion of 4, 5, or 6 amino acids at or near the C-terminus; and (2) a TCE ABS comprising a VH and VL, each having a deletion of 1, 2, or 3 amino acids at or near the N-terminus.

Following cleavage of the protease-cleavable linker the steric hindrance constraining the ability of the TCE ABS to bind to its target is released.

Preferably, the resulting MBM is bispecific for the TAA and the TCE ABS's target (e.g., CD3).

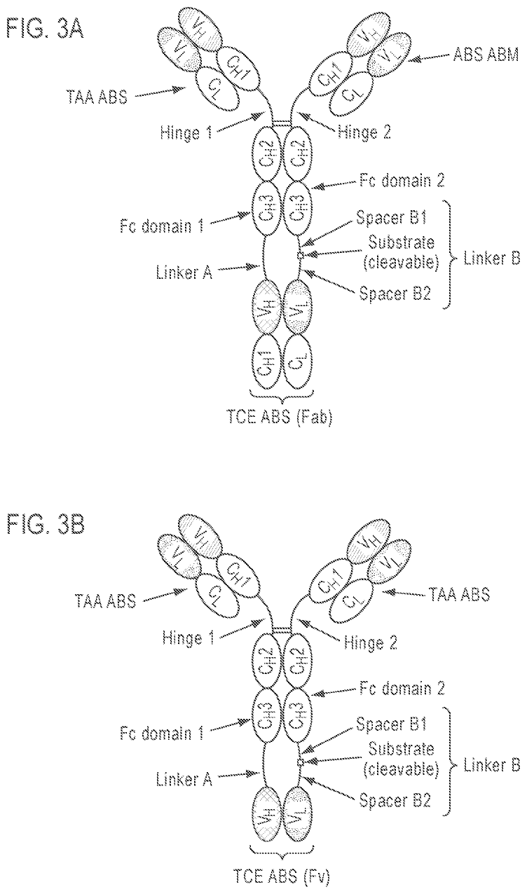

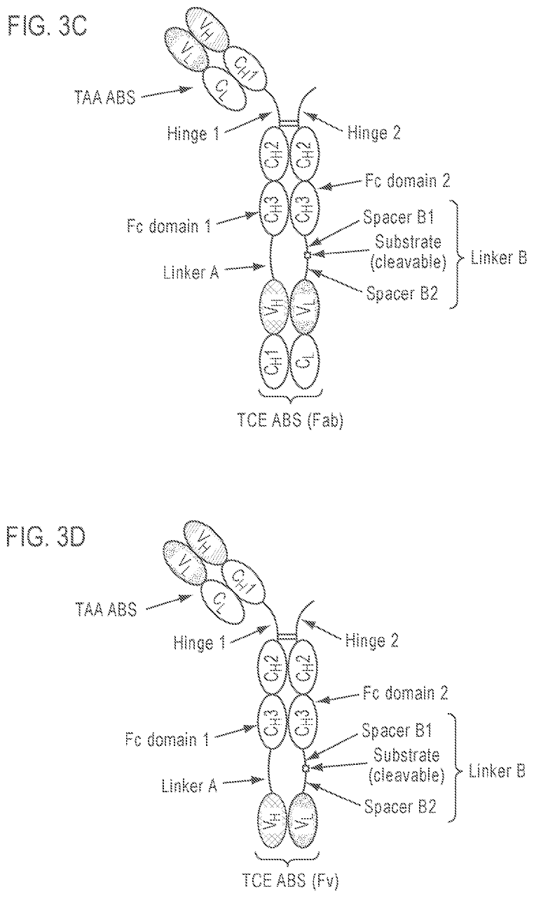

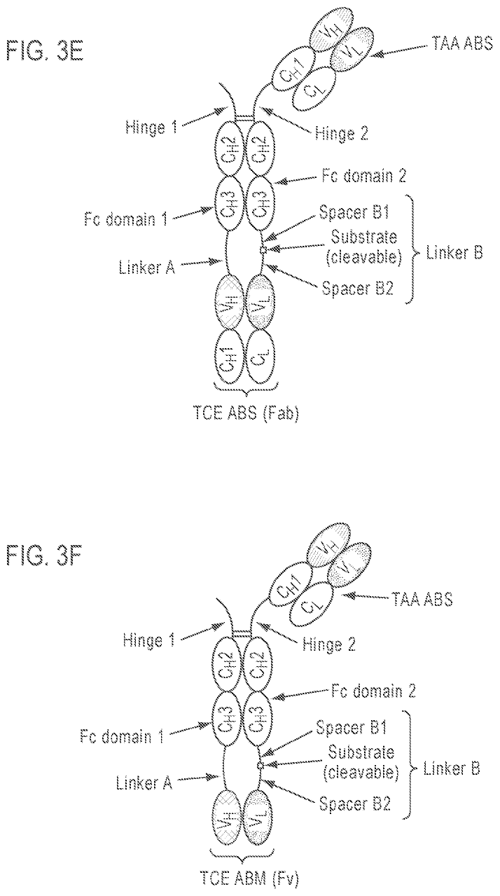

FIGS. 3A to 3F illustrate exemplary embodiments of Type IIA MBM proproteins. As shown in FIGS. 3A and 3B, the Type IIA MBM proproteins can include only a single TAA

ABS. Alternatively, as shown in FIGS. 3C through 3F, Type IIA MBM proproteins can include two TAA ABSs. The depiction of a single substrate and two spacers in the protease-cleavable linker of FIGS. 3A-3G is for illustrative purposes only; as described in Section 6.4, protease-cleavable linkers can include multiple substrates and more than two spacer sequences.

### 6.3.2.2. Type IIB MBM Proproteins

Generally, Type IIB MBM proproteins comprise first polypeptide chain comprising, in an N-terminal to C-terminal orientation, an Fc domain (with an optional hinge domain at its N-terminus), a first protease cleavable linker, an anti-TCE ABS chain, an optional non-cleavable linker and a TAA ABS (or TAA ABS chain) and a second polypeptide chain comprising, in an N-terminal to C-terminal orientation, an Fc domain (with an optional hinge domain at its N-terminus), a protease-cleavable linker, and a TCE ABS chain. When the TAA ABS is composed of multiple polypeptide chains, the MBM proprotein will further comprise the other chain(s), for example the light chain of a Fab. The TCE ABS chains may include a VH and a VL with corresponding CH1 and CL domain domains, such that a TCE ABS in the form of a Fab is formed by the association of the TCE ABS chains. In some embodiments, the VH and/or VL of the TCE ABS chains attached to the Fc domains comprise a deletion of one or more amino acids at or near their N-terminus, e.g., an N-terminal truncation. A VH or VL may have such a deletion precisely at the N-terminus (i.e., may be a truncation), or in some cases within 1, 2, 3, 4, 5, 6, 7, 8, 9, or 10 amino acids of the N-terminus. In some embodiments, the VH of a TCE ABS of a Type IIB MBM proprotein of the present disclosure comprises a deletion (e.g., a truncation) or 1, 2, 3, 4, 5, 6, 7, 8, 9, or 10 amino acids at or near (e.g., within 10 amino acids of) the N-terminus of a reference VH, e.g., a wild-type VH or the VH upon which the TCE ABS is based, such as the VH of an antibody or antibody sequence of Table G. In some embodiments, the VL of a TCE ABS of a Type IIB MBM proprotein of the present disclosure comprises a deletion (e.g., a truncation) of 1, 2, 3, 4, 5, 6, 7, 8, 9, or 10 amino acids at or near (e.g., within 10 amino acids of) the N-terminus of a reference VL, e.g., a wild-type VL or the VL upon which the TCE ABS is based, such as the VL of an antibody or antibody sequence of Table G.

The length of the linkers separating the Fc domains and the TCE ABS chains is chosen so as to optimize the ability of the Fc domains to sterically hinder the TCE ABS from binding its target. Preferably, the length of linkers typically ranges between 5 and 30 amino acids, and in various embodiments is 25 amino acids or less (e.g., ranges between 5 and 25 amino acids), 20 amino acids or less (e.g., ranges between 5 and 20 amino acids), 15 amino acids or less (e.g., ranges between 5 and 15 amino acids), or 10 amino acids or less (e.g., ranges between 5 and 10 amino acids).

One of both Fc domains may, in some embodiments, comprise a deletion of one or more amino acids at or near the C-terminus (e.g., a C-terminal truncation) relative to a wild-type Fc domain. In some embodiments, one or both Fc domains of a Type IIB MBM proprotein of the present disclosure comprise a deletion (e.g., a truncation) of 1, 2, 3, 4, 5, 6, 7, 8, 9, or 10 amino acids at or near the C-terminus relative to a wild-type Fc domain. An Fc domain may have such a deletion precisely at the C-terminus (i.e., may be a truncation), or in some cases within 1, 2, 3, 4, 5, 6, 7, 8, 9, or 10 amino acids of the C-terminus.

In particular embodiments, a Type IIB MBM proprotein of the disclosure comprises (1) Fc domains having a deletion (e.g., a truncation) of 1, 2, 3, 4, 5, or 6 amino acids at or near the C-terminus relative to a wild-type Fc domain; and (2) TCE ABS chains having VH and VL domains with a deletion (e.g., a truncation) of 1, 2, 3, 4, 5, or 6 amino acids at or near the N-terminus. For example, a Type IIB MBM proprotein may comprise (1) two Fc domains, each having a deletion of 4, 5, or 6 amino acids at or near the C-terminus; and (2) a TCE ABS comprising a VH and VL, each having a deletion of 1, 2, or 3 amino acids at or near the N-terminus.

Following cleavage of the protease-cleavable linker an MBM is released comprising the TCE ABS, optional linker and the TAA ABS. When the TAA ABS is composed of multiple polypeptide chains, the resulting MBM will further comprise the other TAA ABS chain, for example the light chain of a Fab.

Preferably, the resulting MBM is bispecific for the TAA and the TCE ABS's target (e.g., CD3).

FIG. 7 illustrates an exemplary embodiment of a Type IIB MBM proprotein. The depiction of a single substrate and two spacers in the protease-cleavable linkers of FIG. 7 is for illustrative purposes only; as described in Section 6.4, protease-cleavable linkers can include multiple substrates and more than two spacer sequences.

### 6.4. Protease Cleavable Linkers

The MBM proproteins of the disclosure typically comprise at least components that are connected to one another by a linker with one or more substrates for a protease and whose cleavage results in activation of the MBM.

A protease-cleavable linker can range from 20 amino acids to 80 or more amino acids, and in certain aspects a non-cleavable peptide linker ranges from 20 amino acids to 60 amino acids, 20 amino acids to 40 amino acids, from 30 amino acids to 50 amino acids, from 20 amino acids to 80 amino acids, or from 30 amino acids to 70 amino acids in length.

The protease cleavable linkers comprise one or more substrate sequences for one or more proteases, for example one or more of the proteases set forth in Section 6.4.1. The one or more substrate sequences, e.g., one or more of the substrate sequences set forth in Section 6.4.2, are typically flanked by one or more spacer sequences, e.g., spacer sequences as described in Section 6.4.3. Each protease-cleavable linker can include one, two, three or more substrate sequences. The spacer sequences can be adjoining, overlapping, or separated by spacer sequences. Preferably, the C- and N-termini of the protease-cleavable linkers contain spacer sequences.

Exemplary protease-cleavable linker sequences ae set forth in Section 6.4.4

### 6.4.1. Proteases

Exemplary protease whose substrate sequences can be incorporated into the protease-cleavable linkers are set forth in Table A below.

TABLE A
Exemplary Proteases for Substrate Cleavage

| | | |
|---|---|---|
| ADAMS, ADAMTS, e.g. | Caspases, e.g., | MMP24 |
| ADAM8 | Caspase 1 | MMP26 |
| ADAM9 | Caspase 2 | MMP27 |
| ADAM10 | Caspase 3 | Cysteine proteinases, e.g., |
| ADAM12 | Caspase 4 | Cruzipain |
| ADAM15 | Caspase 5 | Legumain |
| ADAM17/TACE | Caspase 6 | Otubain-2 |
| ADAMDEC1 | Caspase 7 | KLKs, e.g., |
| ADAMTS1 | Caspase 8 | KLK4 |
| ADAMTS4 | Caspase 9 | KLK5 |
| ADAMTS5 | Caspase 10 | KLK6 |
| Aspartate proteases, e.g., | Caspase 14 | KLK7 |
| BACE | Cysteine cathepsins, e.g., | KLK8 |
| Renin | Cathepsin B | KLK10 |
| Aspartic cathepsins, e.g., | Cathepsin C | KLK11 |
| Cathepsin D | Cathepsin K | KLK13 |
| Cathepsin E | Cathepsin L | KLK14 |
| NS3/4A | Cathepsin S | Metallo proteinases, e.g., |
| PACE4 | Cathepsin V/L2 | Meprin |
| Plasmin | Cathepsin X/Z/P | Neprilysin |
| PSA | MMPs, e.g., | PSMA |
| tPA | MMP1 | BMP-1 |
| Thrombin | MMP2 | Serine proteases, e.g., |
| Tryptase | MMP3 | activated protein C |
| uPA | MMP7 | Cathepsin A |
| Type II Transmembrane | MMP8 | Cathepsin G |
| Serine Proteases (TTSPs), | MMP9 | Chymase |
| e.g., | MMP10 | coagulation factor proteases |
| DESC1 | MMP11 | (e.g., FVIIa, FIXa, FXa, |
| DPP-4 | MMP12 | FXIa, FXIIa) |
| FAP | MMP13 | Human Neutrophil Elastase |
| Hepsin | MMP14 | Lactoferrin |
| Matriptase-2 | MMP15 | |
| MT/SP1/Matriptase | MMP16 | |
| ™PRSS2 | MMP17 | |
| ™PRSS3 | MMP19 | |
| ™PRSS4 | MMP20 | |
| | MMP23 | |

In particular embodiments, the protease is matrix metalloprotease (MMP)-2, MMP-9, legumain asparaginyl endopeptidase, thrombin, fibroblast activation protease (FAP), MMP-1, MMP-3, MMP-7, MMP-8, MMP-12, MMP-13, MMP-14, membrane type 1 matrix metalloprotease (MT1-MMP), plasmin, transmembrane protease, serine (TMPRSS-3/4), cathepsin A, cathepsin B, cathepsin D, cathepsin E, cathepsin F, cathepsin H, cathepsin K, cathepsin L, cathepsin L2, cathepsin O, cathepsin S, caspase 1, caspase 2, caspase 3, caspase 4, caspase 5, caspase 6, caspase 7, caspase 8, caspase 9, caspase 10, caspase 11, caspase 12, caspase 13, caspase 14, human neutrophil elastase, urokinase/urokinase-type plasminogen activator (uPA), a disintegrin and metalloprotease (ADAM) 10, ADAM12, ADAM17, ADAM with thrombospondin motifs (ADAMTS), ADAMTS5, beta secretase (BACE), granzyme A, granzyme B, guanidinobenzoatase, hepsin, matriptase, matriptase 2, meprin, neprilysin, prostate-specific membrane antigen (PSMA), tumor necrosis factor-converting enzyme (TACE), kallikrein-related peptidase (KLK) 3, KLK5, KLK7, KLK11, NS3/4 protease of hepatitis C virus (HCV-NS3/4), tissue plasminogen activator (tPA), calpain, calpain 2, glutamate carboxypeptidase II, plasma kallikrein, AMSH-like protease, AMSH, γ-secretase component, antiplasmin cleaving enzyme (APCE), decysin 1, apoptosis-related cysteine peptidase, or N-acetylated alpha-linked acidic dipeptidase-like 1.

### 6.4.2. Substrates

Exemplary substrate sequences that are cleavable by a tumor protease and can be incorporated into the protease-cleavable linkers are set forth in Table B below.

TABLE B
Substrate Sequences for Protease Cleavable Linkers

| Substrate Sequence | Designation | Cleaving Protease |
|---|---|---|
| (DE)₈RPLALWRS(DR)₈ (SEQ ID NO: 2) | SU1 | MMP7 |
| AARGPAIH (SEQ ID NO: 3) | SU2 | |
| AAYHLVSQ (SEQ ID NO: 4) | SU3 | Collagenase |
| AGLGISST (SEQ ID NO: 5) | SU4 | Collagenase |
| AGLGVVER (SEQ ID NO: 6) | SU5 | Collagenase |
| ALAL (SEQ ID NO: 7) | SU6 | Lysosomal Enzyme |
| ALFFSSPP (SEQ ID NO: 8) | SU7 | |
| ALFKSSFP (SEQ ID NO: 9) | SU8 | |
| ALLLALL (SEQ ID NO: 10) | SU9 | TOP |
| AQFVLTEG (SEQ ID NO: 11) | SU10 | Collagenase |
| AQNLLGMV (SEQ ID NO: 12) | SU11 | |
| AVGLLAPP (SEQ ID NO: 13) | SU12 | Serine protease |
| DAFK (SEQ ID NO: 14) | SU13 | Urokinase plasminogen |
| | | activator (uPA) |
| DEVD (SEQ ID NO: 15) | SU14 | Caspase-3 |
| DEVDP (SEQ ID NO: 16) | SU15 | Caspase-3 |
| DPRSFL (SEQ ID NO: 17) | SU16 | Thrombin |
| DVAQFVLT (SEQ ID NO: 18) | SU17 | Collagenase |
| DVLK (SEQ ID NO: 19) | SU18 | Plasmin |
| DWLYWPGI (SEQ ID NO: 20) | SU19 | |
| EDDDDKA (SEQ ID NO: 21) | SU20 | Enterokinase |
| EP(Cit)G(Hof)YL (SEQ ID NO: 22) | SU21 | MMP2, MMP9, MMP14 |
| EPQALAMS (SEQ ID NO: 23) | SU22 | Collagenase |
| ESLPWVAV (SEQ ID NO: 24) | SU23 | Collagenase |
| ESPAYYTA (SEQ ID NO: 25) | SU24 | MMP |
| F(Pip)RS | SU25 | Thrombin |
| FK | SU26 | Lysosomal Enzyme |
| FPRPLGITGL (SEQ ID NO: 27) | SU27 | |
| FRLLDWQW (SEQ ID NO: 28) | SU28 | |
| GFLG (SEQ ID NO: 29) | SU29 | Lysosomal Enzyme |
| GGAANLVRGG (SEQ ID NO: 30) | SU30 | MMP11 |
| GGGRR (SEQ ID NO: 31) | SU31 | Urokinase plasminogen |
| | | activator (uPA) |
| GGPRGLPG (SEQ ID NO: 32) | SU32 | Cathepsin K |
| GGQPSGMWGW (SEQ ID NO: 33) | SU33 | |
| GGSIDGR (SEQ ID NO: 34) | SU34 | Factor Xa |
| GGWHTGRN (SEQ ID NO: 35) | SU35 | |
| GIAGQ (SEQ ID NO: 36) | SU36 | Collagenase |
| GKAFRR (SEQ ID NO: 37) | SU37 | Kallikrein 2 |
| GPAGLYAQ (SEQ ID NO: 38) | SU38 | |
| GPAGMKGL (SEQ ID NO: 39) | SU39 | |
| GPEGLRVG (SEQ ID NO: 40) | SU40 | Collagenase |
| GPLGIAGI (SEQ ID NO: 41) | SU41 | Collagenase |
| GPLGVRG (SEQ ID NO: 42) | SU42 | |
| GPQGIAGQ (SEQ ID NO: 43) | SU43 | Collagenase |
| GPQGLLGA (SEQ ID NO: 44) | SU44 | Collagenase |
| GPRSFG (SEQ ID NO: 45) | SU45 | |
| GPRSFGL (SEQ ID NO: 46) | SU46 | |
| GPSHLVLT (SEQ ID NO: 47) | SU47 | |
| GVSQNYPIVG (SEQ ID NO: 48) | SU48 | HIV Protease |
| GVVQASCRLA (SEQ ID NO: 49) | SU49 | CMV Protease |
| GWEHDG (SEQ ID NO: 50) | SU50 | Interleukin 1β converting |
| | | enzyme |
| HSSKLQ (SEQ ID NO: 51) | SU51 | Prostate Specific Antigen |
| HSSKLQEDA (SEQ ID NO: 52) | SU52 | Prostate Specific Antigen |
| HSSKLQL (SEQ ID NO: 53) | SU53 | Prostate Specific Antigen |
| HTGRSGAL (SEQ ID NO: 54) | SU54 | |
| IDGR (SEQ ID NO: 55) | SU55 | Factor Xa |
| IEGR (SEQ ID NO: 56) | SU56 | Factor Xa |
| ILPRSPAF (SEQ ID NO: 57) | SU57 | |
| IPVSLRSG (SEQ ID NO: 58) | SU58 | MMP |
| ISSGL (SEQ ID NO: 59) | SU59 | MMP |
| ISSGLL (SEQ ID NO: 60) | SU60 | MMP |
| ISSGLLS (SEQ ID NO: 61) | SU61 | MMP |
| ISSGLLSS (SEQ ID NO: 62) | SU62 | MMP |
| ISSGLSS (SEQ ID NO: 63) | SU63 | MMP |
| KGSGDVEG (SEQ ID NO: 64) | SU64 | Caspase-3 |
| KQEQNPGST (SEQ ID NO: 65) | SU65 | FAP |
| KRALGLPG (SEQ ID NO: 66) | SU66 | MMP7 |
| LAAPLGLL (SEQ ID NO: 67) | SU67 | |
| LAPLGLQRR (SEQ ID NO: 68) | SU68 | |
| LAQKLKSS (SEQ ID NO: 69) | SU69 | |
| LAQRLRSS (SEQ ID NO: 70) | SU70 | |
| LEATA (SEQ ID NO: 71) | SU71 | MMP9 |
| LKAAPRWA (SEQ ID NO: 72) | SU72 | |
| LLAPSHRA (SEQ ID NO: 73) | SU73 | |
| LPGGLSPW (SEQ ID NO: 74) | SU74 | |
| LSGRSANI (SEQ ID NO: 75) | SU75 | Serine protease |
| LSGRSANP (SEQ ID NO: 76) | SU76 | Serine protease |
| LSGRSDDH (SEQ ID NO: 77) | SU77 | Serine protease |
| LSGRSDIH (SEQ ID NO: 78) | SU78 | Serine protease |
| LSGRSDNH (SEQ ID NO: 79) | SU79 | Serine protease |
| LSGRSDNI (SEQ ID NO: 80) | SU80 | Serine protease |
| LSGRSDNP (SEQ ID NO: 81) | SU81 | Serine protease |
| LSGRSDQG (SEQ ID NO: 82) | SU82 | Serine protease |
| LSGRSDQH (SEQ ID NO: 83) | SU83 | Serine protease |
| LSGRSDTH (SEQ ID NO: 84) | SU84 | Serine protease |
| LSGRSDYH (SEQ ID NO: 85) | SU85 | Serine protease |
| LSGRSGNH (SEQ ID NO: 86) | SU86 | Serine protease |
| LVLASSSFGY (SEQ ID NO: 87) | SU87 | Herpes Simplex Virus |
| | | Protease |
| MDAFLESS (SEQ ID NO: 88) | SU88 | Collagenase |
| MGLFSEAG (SEQ ID NO: 89) | SU89 | |
| MIAPVAYR (SEQ ID NO: 90) | SU90 | |
| MVLGRSLL (SEQ ID NO: 91) | SU91 | |
| NLL | SU92 | Cathepsin B |
| NTLSGRSENHSG (SEQ ID NO: 92) | SU93 | |
| NTLSGRSGNHGS (SEQ ID NO: 93) | SU94 | |
| PAGLWLDP (SEQ ID NO: 94) | SU95 | |
| PGGPAGIG (SEQ ID NO: 95) | SU96 | |
| PIC(Et)FF (SEQ ID NO: 96) | SU97 | Cathepsin D |
| PLGC(me)AG (SEQ ID NO: 97) | SU98 | MMP |
| PLGL (SEQ ID NO: 98) | SU99 | |
| PLGLAG (SEQ ID NO: 99) | SU100 | MMP |
| PLGLAX (SEQ ID NO: 100) | SU101 | MMP |
| PLGLWA (SEQ ID NO: 101) | SU102 | MMP |
| PLGLWSQ (SEQ ID NO: 102) | SU103 | MMP |
| PLTGRSGG (SEQ ID NO: 103) | SU104 | |
| PMAKK (SEQ ID NO: 104) | SU105 | |
| PPRSFL (SEQ ID NO: 105) | SU106 | Thrombin |
| PR(S/T)(L/I)(S/T) | SU107 | MMP9 |
| PRFRIIGG (SEQ ID NO: 107) | SU108 | Plasmin |
| PVGYTSSL (SEQ ID NO: 108) | SU109 | |
| PVQPIGPQ (SEQ ID NO: 109) | SU110 | Collagenase |
| QALAMSAI (SEQ ID NO: 110) | SU111 | Collagenase |
| QGRAITFI (SEQ ID NO: 111) | SU112 | |
| QNQALRMA (SEQ ID NO: 112) | SU113 | |
| RGPA (SEQ ID NO: 113) | SU114 | |
| RGPAFNPM (SEQ ID NO: 114) | SU115 | |
| RGPATPIM (SEQ ID NO: 115) | SU116 | |
| RKSSIIIRMRDVVL (SEQ ID NO: 116) | SU117 | Plasmin |
| RLQLKAC (SEQ ID NO: 117) | SU118 | MMP |
| RLQLKL (SEQ ID NO: 118) | SU119 | MMP |
| RMHLRSLG (SEQ ID NO: 119) | SU120 | |
| RPSPMWAY (SEQ ID NO: 120) | SU121 | |
| RQARVVNG (SEQ ID NO: 121) | SU122 | Matripase |
| SAGFSLPA (SEQ ID NO: 122) | SU123 | |
| SAPAVESE (SEQ ID NO: 123) | SU124 | Collagenase |
| SARGPSRW (SEQ ID NO: 124) | SU125 | |
| SGEPAYYTA (SEQ ID NO: 125) | SU126 | |
| SGGPLGVR (SEQ ID NO: 126) | SU127 | |
| SGRIGFLRTA (SEQ ID NO: 127) | SU128 | MMP14 |
| SGRSA (SEQ ID NO: 128) | SU129 | Urokinase plasminogen |
| | | activator (uPA) |
| SGRSANPRG (SEQ ID NO: 129) | SU130 | |
| SMLRSMPL (SEQ ID NO: 130) | SU131 | |
| SPLPLRVP (SEQ ID NO: 131) | SU132 | |
| SPLTGRSG (SEQ ID NO: 132) | SU133 | |
| SPRSIMLA (SEQ ID NO: 133) | SU134 | |
| SSRGPAYL (SEQ ID NO: 134) | SU135 | |
| SSRHRRALD (SEQ ID NO: 135) | SU136 | Plasmin |
| SSSFDKGKYKKGDDA (SEQ ID NO: 136) | SU137 | Plasmin |
| SSSFDKGKYKRGDDA (SEQ ID NO: 137) | SU138 | Plasmin |
| STFPFGMF (SEQ ID NO: 138) | SU139 | |
| TARGPSFK (SEQ ID NO: 139) | SU140 | |
| TGRGPSWV (SEQ ID NO: 140) | SU141 | |
| TSGRSANP (SEQ ID NO: 141) | SU142 | |
| TSTSGRSANPRG (SEQ ID NO: 142) | SU143 | |
| VAGRSMRP (SEQ ID NO: 143) | SU144 | |
| VAQFVLTE (SEQ ID NO: 144) | SU145 | Collagenase |
| VHMPLGFLGP (SEQ ID NO: 145) | SU146 | |
| VPLSLYSG (SEQ ID NO: 146) | SU147 | MMP9 |
| WPEGRRS (SEQ ID NO: 147) | SU148 | |
| WATPRPMR (SEQ ID NO: 148) | SU149 | |
| YGAGLGW (SEQ ID NO: 149) | SU150 | Collagenase |
| HPVGLLAR (SEQ ID NO:260) | SU151 | |

### 6.4.3. Spacers

Exemplary spacer sequences that can be incorporated into the protease-cleavable linkers are set forth in Table C below. In addition to the spacer sequences set forth in Table C, any of the non-cleavable linker sequences described in Section 6.5, e.g., the non-cleavable linker sequences set forth in Table E, or portions thereof can be used as spacer sequences.

TABLE C
Spacer Sequences for Protease Cleavable Linkers

| Spacer Sequence | Designation |
|---|---|
| (GGGGS)ₙ (SEQ ID NO: 150) | SP1 |
| (GGGS)ₙ (SEQ ID NO: 151) | SP2 |
| (GGS)ₙ | SP3 |
| (GS)ₙ | SP4 |
| (GSGGS)ₙ (SEQ ID NO: 154) | SP5 |
| GGGGSGGGGS (SEQ ID NO: | SP6 |
| 155) | |
| GGGGSGGGGSGGGGS (SEQ | SP7 |
| ID NO: 156) | |
| GGGGSGGGGSGGGGSGGGGS | SP8 |
| (SEQ ID NO: 157) | |
| GGGKSGGGKSGGGKS (SEQ ID | SP9 |
| NO: 158) | |
| GGGKSGGKGSGKGGS (SEQ ID | SP10 |
| NO: 159) | |
| GGGS (SEQ ID NO: 160) | SP11 |
| GGGSG (SEQ ID NO: 161) | SP12 |
| GGKGSGGKGSGGKGS (SEQ ID | SP13 |
| NO: 162) | |
| GGSGGGGSGGGGS (SEQ ID | SP14 |
| NO: 163) | |
| GGSGGS (SEQ ID NO: 164) | SP15 |
| GGSGGSGGSGS (SEQ ID NO: | SP16 |
| 165) | |
| GSGGG (SEQ ID NO: 166) | SP17 |
| GSGSG (SEQ ID NO: 167) | SP18 |
| GSS | SP19 |
| GSSG (SEQ ID NO: 168) | SP20 |
| GSSGGSGGSG (SEQ ID NO: | SP21 |
| 169) | |
| GSSGGSGGSGG (SEQ ID NO: | SP22 |
| 170) | |
| GSSGGSGGSGGS (SEQ ID NO: | SP23 |
| 171) | |
| GSSGGSGGSGGSG (SEQ ID | SP24 |
| NO: 172) | |
| GSSGGSGGSGGSGGGS (SEQ | SP25 |
| ID NO: 173) | |
| GSSGGSGGSGS (SEQ ID NO: | SP26 |
| 174) | |
| GSSGT (SEQ ID NO: 175) | SP27 |
| GSSSG (SEQ ID NO: 176) | SP28 |
| QGQSGQ (SEQ ID NO: 177) | SP29 |
| QGQSGQG (SEQ ID NO: 178) | SP30 |
| QGQSGS (SEQ ID NO: 179) | SP31 |
| QSGQ (SEQ ID NO: 180) | SP32 |
| QSGQG (SEQ ID NO: 181) | SP33 |
| QSGS (SEQ ID NO: 182) | SP34 |
| SGQ | SP35 |
| SGQG (SEQ ID NO: 183) | SP36 |
| SGS | SP37 |
| (G)ₙ | SP38 |

In some embodiments, as used in Table C above, n is an integer from 1 to 10, e.g., 1, 2, 3, 4, 5, 6, 7, 8, 9 or 10.

### 6.4.4. Exemplary Protease Cleavable Linkers

Exemplary protease-cleavable linkers comprising one or more substrate sequences as well as spacer sequences are set forth in Table D below.

TABLE D
Protease Cleavable Linker Sequences

| Linker Sequence | Designation | Cleaving Protease(s) |
|---|---|---|
| GGGISSGLLSGRSDNHGGGISSG | PCL1 | |
| LLSGRSDNHGGS (SEQ ID NO: | | |
| 184) | | |
| GGGISSGLLSGRSDNHGGGISSG | PCL2 | |
| LLSGRSDNHGGS | | |
| GGGISSGLLSGRSDNHGGGISSG | | |
| LLSGRSDNHGGS (SEQ ID NO: | | |
| 185) | | |
| GGSGGSIPVSLRSGGGISSGLLS | PCL3 | |
| GRSDNHGGSGGS (SEQ ID NO: | | |
| 186) | | |
| GGSGGSVPLSLYSGGGISSGLLS | PCL4 | |
| GRSDNHGGSGGS (SEQ ID NO: | | |
| 187) | | |
| GGSHPVGLLARGGGHPVGLLAR | PCL5 | |
| GGGHPVGLLARGS (SEQ ID NO: | | |
| 188) | | |
| GGSHPVGLLARGGGHPVGLLAR | PCL6 | |
| GGSGRSAGGSGRSA (SEQ ID | | |
| NO: 189) | | |
| AVGLLAPPGGLSGRSANI (SEQ ID | PCL7 | ADAM17_2, FAPa_1, CTSL1_1 |
| NO: 190) | | |
| AVGLLAPPGGLSGRSANP (SEQ | PCL8 | FAPa_1, ADAM17_2, CTSL1_1 |
| ID NO: 191) | | |
| AVGLLAPPGGLSGRSDDH (SEQ | PCL9 | MMP14_1, MMP14_1, MMP14_1 |
| ID NO: 192) | | |
| AVGLLAPPGGLSGRSDIH (SEQ | PCL10 | MMP14_1, MMP14_1, MMP14_1 |
| ID NO: 193) | | |
| AVGLLAPPGGLSGRSDNH (SEQ | PCL11 | MMP14_1, MMP14_1 |
| ID NO: 194) | | |
| AVGLLAPPGGLSGRSDNI (SEQ | PCL12 | MMP14_1, CTSL1_1, ADAM17_2 |
| ID NO: 195) | | |
| AVGLLAPPGGLSGRSDNP (SEQ | PCL13 | CTSL1_1, ADAM17_2, FAPa_1 |
| ID NO: 196) | | |
| AVGLLAPPGGLSGRSDQH (SEQ | PCL14 | |
| ID NO: 197) | | |
| AVGLLAPPGGLSGRSDTH (SEQ | PCL15 | FAPa_1, CTSL1_1, ADAM17_2 |
| ID NO: 198) | | |
| AVGLLAPPGGLSGRSDYH (SEQ | PCL16 | |
| ID NO: 199) | | |
| AVGLLAPPGGTSTSGRSANPRG | PCL17 | |
| (SEQ ID NO: 200) | | |
| AVGLLAPPSGRSANPRG (SEQ ID | PCL18 | |
| NO: 201) | | |
| AVGLLAPPTSGRSANPRG (SEQ | PCL19 | |
| ID NO: 202) | | |
| GGALFKSSFPGPAGLYAQPLAQK | PCL20 | CTSL1_1, MMP14_1, ADAM17_2 |
| LKSSGGK (SEQ ID NO: 203) | | |
| GGGGSGGGGSGGGGSFVGGTG | PCL21 | |
| GGGSGGGGSGGS (SEQ ID NO: | | |
| 204) | | |
| GGGGSGGGGSGGGGSISSGLLS | PCL22 | |
| GRSDNHGGSGGS (SEQ ID NO: | | |
| 205) | | |
| GGGGSGGGGSGGGGSVPLSLYS | PCL23 | |
| GGGSGGSGGSGS (SEQ ID NO: | | |
| 206) | | |
| GGGGSGGGGSGPLGLWSQGGG | PCL24 | |
| GSGGGGSGGGGSGG (SEQ ID | | |
| NO: 207) | | |
| GGGGSGGGGSKKAAPGGGGSG | PCL25 | |
| GGGSGGGGSGGS (SEQ ID NO: | | |
| 208) | | |
| GGGGSGGGGSKKAAPVNGGGG | PCL26 | |
| GSGGGGGGGGS (SEQ ID NO: | | |
| 209) | | |
| GGGGSGGGGSPMAKKGGGGSG | PCL27 | |
| GGGSGGGGSGGS (SEQ ID NO: | | |
| 210) | | |
| GGGGSGGGGSPMAKKVNGGGG | PCL28 | |
| GSGGGGSGGGGS (SEQ ID NO: | | |
| 211) | | |
| GGGGSGGGGSQARAKGGGGSG | PCL29 | |
| GGGSGGGGSGGS (SEQ ID NO: | | |
| 212) | | |
| GGGGSGGGGSQARAKVNGGGG | PCL30 | |
| GSGGGGSGGGGS (SEQ ID NO: | | |
| 213) | | |
| GGGGSGGGGSRQARVVNGGGG | PCL31 | |
| GSGGGGSGGGGS (SEQ ID NO: | | |
| 214) | | |
| GGGGSGGGGSRQARVVNGGGG | PCL32 | |
| GSVPLSLYSGGGGGSGGGGS | | |
| (SEQ ID NO: 215) | | |
| GGGGSGGGGSRQARVVNSVPLS | PCL33 | |
| LYSGGGGGSGGGGS (SEQ ID | | |
| NO: 216) | | |
| GGGGSGGGGSVHMPLGFLGPG | PCL34 | |
| GGGSGGGGSGGS (SEQ ID NO: | | |
| 217) | | |
| GGGGSVHMPLGFLGPGRSRGSF | PCL35 | |
| PGGGGS (SEQ ID NO: 218) | | |
| GGGGSVHMPLGFLGPPMAKKGG | PCL36 | |
| GGSGGGGSGGS (SEQ ID NO: | | |
| 219) | | |
| GGGGSVHMPLGFLGPRQARVVN | PCL37 | |
| GGGGSGGGGS (SEQ ID NO: | | |
| 220) | | |
| GGGGSVHMPLGFLGPRQARVVN | PCL38 | |
| GGGGSGGGGSGG (SEQ ID NO: | | |
| 221) | | |
| GGPLAQKLKSSALFKSSFPGPAG | PCL39 | ADAM17_2, CTSL1_1, MMP14_1 |
| LYAQGGR (SEQ ID NO: 222) | | |
| GLSGRSDNHGGAVGLLAPP | PCL40 | |
| (SEQ ID NO: 223) | | |
| GLSGRSDNHGGVHMPLGFLGP | PCL41 | |
| (SEQ ID NO: 224) | | |
| ISSGLLSGRSANI (SEQ ID NO: | PCL42 | MMP, Serine protease |
| 225) | | |
| ISSGLLSGRSANP (SEQ ID NO: | PCL43 | MMP, Serine protease |
| 226) | | |
| ISSGLLSGRSANPRG (SEQ ID | PCL44 | MMP, Serine protease |
| NO: 227) | | |
| ISSGLLSGRSDDH (SEQ ID NO: | PCL45 | MMP, Serine protease |
| 228) | | |
| ISSGLLSGRSDIH (SEQ ID NO: | PCL46 | MMP, Serine protease |
| 229) | | |
| ISSGLLSGRSDNH (SEQ ID NO: | PCL47 | MMP, Serine protease |
| 230) | | |
| ISSGLLSGRSDNI (SEQ ID NO: | PCL48 | CTSL1_1, MMP14_1 |
| 231) | | |
| ISSGLLSGRSDNP (SEQ ID NO: | PCL49 | MMP, Serine protease |
| 232) | | |
| ISSGLLSGRSDQH (SEQ ID NO: | PCL50 | MMP, Serine protease |
| 233) | | |
| ISSGLLSGRSDTH (SEQ ID NO: | PCL51 | MMP, Serine protease |
| 234) | | |
| ISSGLLSGRSDYH (SEQ ID NO: | PCL52 | MMP, Serine protease |
| 235) | | |
| ISSGLLSGRSGNH (SEQ ID NO: | PCL53 | MMP, Serine protease |
| 236) | | |
| ISSGLLSSGGSGGSLSGRSDNH | PCL54 | |
| (SEQ ID NO: 237) | | |
| ISSGLLSSGGSGGSLSGRSGNH | PCL55 | |
| (SEQ ID NO: 238) | | |
| KGGPGGPAGIGPLAQRLRSSALF | PCL56 | FAPa_1, ADAM17_1, CTSL1_1 |
| KSSFPGR (SEQ ID NO: 239) | | |
| KSGPGGPAGIGALFFSSPPLAQKL | PCL57 | FAPa_1, CTSL1_2, ADAM17_2 |
| KSSGGR (SEQ ID NO: 240) | | |
| LSGRSDNHGGAVGLLAPP (SEQ | PCL58 | |
| ID NO: 241) | | |
| LSGRSDNHGGSGGSISSGLLSS | PCL59 | |
| (SEQ ID NO: 242) | | |
| LSGRSDNHGGSGGSQNQALRMA | PCL60 | |
| (SEQ ID NO: 243) | | |
| LSGRSDNHGGVHMPLGFLGP | PCL61 | |
| (SEQ ID NO: 244) | | |
| LSGRSGNHGGSGGSISSGLLSS | PCL62 | |
| (SEQ ID NO: 245) | | |
| LSGRSGNHGGSGGSQNQALRMA | PCL63 | |
| (SEQ ID NO: 246) | | |
| QNQALRMAGGSGGSLSGRSDNH | PCL64 | |
| (SEQ ID NO: 247) | | |
| QNQALRMAGGSGGSLSGRSGNH | PCL65 | |
| (SEQ ID NO: 248) | | |
| RGGALFKSSFPLAQKLKSSGPAG | PCL66 | CTSL1_1, ADAM17_2, MMP14_1 |
| LYAQGGK (SEQ ID NO: 249) | | |
| RGGGPAGLYAQPLAQKLKSSALF | PCL67 | MMP14_1, ADAM17_2, CTSL1_1 |
| KSSFPGG (SEQ ID NO: 250) | | |
| SGGFPRSGGSFNPRTFGSKRKR | PCL68 | thrombin, factor Xa, hepsin |
| RGSRGGGG (SEQ ID NO: 251) | | |
| SGPLAQKLKSSGPAGLYAQALFK | PCL69 | ADAM17_2, MMP14_1, CTSL1_1 |
| SSFPGSK (SEQ ID NO: 252) | | |
| TSTSGRSANPRGGGAVGLLAPP | PCL70 | |
| (SEQ ID NO: 253) | | |
| TSTSGRSANPRGGGVHMPLGFL | PCL71 | |
| GP (SEQ ID NO: 254) | | |
| VHMPLGFLGPGGLSGRSDNH | PCL72 | |
| (SEQ ID NO: 255) | | |
| VHMPLGFLGPGGTSTSGRSANP | PCL73 | |
| RG (SEQ ID NO: 256) | | |
| SGRSAGGGSGRSAGGGSGRSA | PCL74 | uPA |
| (SEQ ID NO: 257) | | |
| HPVGLLARGGGHPVGLLARGGG | PCL75 | MPA (MMP-2 and uPA) |
| SGRSAGGGSGRSA (SEQ ID NO: | | |
| 258) | | |
| GPLGVRGK (SEQ ID NO: 259) | PCL76 | MMP-2 |
| HPVGLLAR (SEQ ID NO: 260) | PCL77 | MMP-2 |
| GPQGIAGQ (SEQ ID NO: 43) | PCL78 | MMP-2, MMP-9, and to some degree |
| | | MT 1-MMP |
| VPMSMRGG (SEQ ID NO: 261) | PCL79 | MMP-9 and MMP-2 |
| IPVSLRSG (SEQ ID NO: 58) | PCL80 | MMP-2, and to some degree MMP-9 or |
| | | MMP-7 |
| RPFSMIMG (SEQ ID NO: 262) | PCL81 | MMP-9 and MMP-7, to some degree |
| | | MMP-3 |
| VPLSLTMG (SEQ ID NO: 263) | PCL82 | MMP-7, to some degree MMP-9, MMP- |
| | | 2, MPT-1-MMP |
| VPLSLYSG (SEQ ID NO: 146) | PCL83 | MMP-2, MMP-9, MMP-7 |
| IPESLRAG (SEQ ID NO: 264) | PCL84 | MMP-2, MMP-7, MMP-9, to some |
| | | degree MPT-1-MMP |
| GGGISSGLLSGRSDNHGGGS | PCL85 | |
| (SEQ ID NO: 339) | | |
| GGGHPVGLLARGGGS (SEQ ID | PCL86 | |
| NO: 340) | | |
| GGGSGGGSGGGGISSGLLSGRS | PCL87 | |
| DNHGGGSGGGSGGS (SEQ ID | | |
| NO: 341) | | |
| GGGGISSGLLSGRSDNHGGGISS | PCL88 | |
| GLLSGRSDNHGGS (SEQ ID NO: | | |
| 342) | | |
| GGGSGGSIPVSLRSGGGISSGLL | PCL89 | |
| SGRSDNHGGSGGS (SEQ ID NO: | | |
| 343) | | |
| GGGSGGSVPLSLYSGGGISSGLL | PCL90 | |
| SGRSDNHGGSGGS (SEQ ID NO: | | |
| 344) | | |
| GGGSHPVGLLARGGGHPVGLLA | PCL91 | |
| RGGGHPVGLLARGS (SEQ ID | | |
| NO: 345) | | |
| GGGSHPVGLLARGGGHPVGLLA | PCL92 | |
| RGGSGRSAGGSGRS (SEQ ID | | |
| NO: 346) | | |

In certain aspects, the protease-cleavable linker comprises an amino acid sequence having up to 5, up to 4, up to 3, up to 2 or up to 1 amino acid substitution(s) as compared to the sequence set forth in Table D. Thus, in some embodiments, the protease cleavable linker comprises or consists of any amino acid sequence in Table D with 1-5 amino acid substitutions as compared to the sequence set forth in Table D.

### 6.5. Non-Cleavable Linkers

In certain aspects, the present disclosure provides MBM proproteins in which two or more components of an MBM proprotein are connected to one another by a peptide linker. By way of example and not limitation, linkers can be used to connect (a) an ABS and an Fc domain; (b) two ABSs; or (c) different domains within an ABS (e.g., VH and VL domains in an scFv).

Preferably, all linkers in the MBM proprotein other than the protease-cleavable linker whose cleavage results in activation of the MBM are non-cleavable linkers (NCLs).

A non-cleavable linker can range from 2 amino acids to 60 or more amino acids, and in certain aspects a non-cleavable peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length.

In particular aspects, a non-cleavable linker is at least 5 amino acids, at least 6 amino acids or at least 7 amino acids in length and optionally is up to 30 amino acids, up to 40 amino acids, up to 50 amino acids or up to 60 amino acids in length.

In some embodiments of the foregoing, the non-cleavable linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the non-cleavable linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the non-cleavable linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

Charged (e.g., charged hydrophilic linkers) and/or flexible non-cleavable linkers are particularly preferred.

Examples of flexible non-cleavable linkers that can be used in the MBM proproteins of the disclosure include those disclosed by Chen et al., 2013, Adv Drug Deliv Rev. 65 (10): 1357-1369 and Klein et al., 2014, Protein Engineering, Design & Selection 27 (10): 325-330.

Particularly useful flexible non-cleavable linkers are or comprise repeats of glycines and serines, e.g., a monomer or multimer of GₙS (SEQ ID NO: 265) or SGₙ (SEQ ID NO: 266), where n is an integer from 1 to 10, e.g., 12, 3, 4, 5, 6, 7, 8, 9 or 10. In one embodiment, the non-cleavable linker is or comprises a monomer or multimer of repeat of G4S (SEQ ID NO: 1) e.g., (GGGGS)ₙ (SEQ ID NO: 150).

Polyglycine non-cleavable linkers can suitably be used in the MBM proproteins of the disclosure. In some embodiments, a peptide non-cleavable linker comprises two consecutive glycines (2Gly), three consecutive glycines (3Gly), four consecutive glycines (4Gly) (SEQ ID NO: 267), five consecutive glycines (5Gly) (SEQ ID NO: 268), six consecutive glycines (6Gly) (SEQ ID NO: 269), seven consecutive glycines (7Gly) (SEQ ID NO: 270), eight consecutive glycines (8Gly) (SEQ ID NO: 271) or nine consecutive glycines (9Gly) (SEQ ID NO: 272).

Exemplary non-cleavable linker sequences are set forth in Table E below.

TABLE E
Non-Cleavable Linker Sequences

| Linker Sequence | Designation | SEQ ID NO: |
|---|---|---|
| (GGGGS)ₙ (SEQ ID NO: 150) | NCL1 | |
| (GGGS)ₙ (SEQ ID NO: 151) | NCL2 | |
| (GGS)ₙ | NCL3 | |
| (GS)ₙ | NCL4 | |
| (GSGGS)ₙ (SEQ ID NO: 154) | NCL5 | |
| ADAAP (SEQ ID NO: 273) | NCL6 | |
| ADAAPTVSIFP (SEQ ID NO: 274) | NCL7 | |
| ADAAPTVSIFPP (SEQ ID NO: 275) | NCL8 | |
| AKTTAP (SEQ ID NO: 276) | NCL9 | |
| AKTTAPSVYPLAP (SEQ ID NO: 277) | NCL10 | |
| AKTTPKLEEGEFSEARV (SEQ ID NO: | NCL11 | |
| 278) | | |
| AKTTPKLGG (SEQ ID NO: 279) | NCL12 | |
| AKTTPP (SEQ ID NO: 280) | NCL13 | |
| AKTTPPSVTPLAP (SEQ ID NO: 281) | NCL14 | |
| ASTKGP (SEQ ID NO: 282) | NCL15 | |
| ASTKGPSVFPLAPASTKGPSVFPLAP | NCL16 | |
| (SEQ ID NO: 283) | | |
| EGKSSGSGSESKST (SEQ ID NO: 284) | NCL17 | |
| GEGESGEGESGEGES (SEQ ID NO: | NCL18 | |
| 285) | | |
| GEGESGEGESGEGESGEGES (SEQ ID | NCL19 | |
| NO: 286) | | |
| GEGGSGEGGSGEGGS (SEQ ID NO: 287) | NCL20 | |
| GENKVEYAPALMALS (SEQ ID NO: 288) | NCL21 | |
| GGEGSGGEGSGGEGS (SEQ ID NO: | NCL22 | |
| 289) | | |
| GGGESGGEGSGEGGS (SEQ ID NO: | NCL23 | |
| 290) | | |
| GGGESGGGESGGGES (SEQ ID NO: | NCL24 | |
| 291) | | |
| GGGGSGGGGS (SEQ ID NO: 155) | NCL25 | |
| GGGGSGGGGSGGGGS (SEQ ID NO: | NCL26 | |
| 156) | | |
| GGGGSGGGGSGGGGSGGGGS (SEQ | NCL27 | |
| ID NO: 157) | | |
| GGGKSGGGKSGGGKS (SEQ ID NO: | NCL28 | |
| 158) | | |
| GGGKSGGKGSGKGGS (SEQ ID NO: | NCL29 | |
| 159) | | |
| GGGS (SEQ ID NO: 160) | NCL30 | |
| GGGSG (SEQ ID NO: 161) | NCL31 | |
| GGKGSGGKGSGGKGS (SEQ ID NO: | NCL32 | |
| 162) | | |
| GGS | NCL33 | |
| GGSG (SEQ ID NO: 292) | NCL34 | |
| GGSGG (SEQ ID NO: 293) | NCL35 | |
| GGSGG (SEQ ID NO: 293) | NCL36 | |
| GGSGGGGSG (SEQ ID NO: 294) | NCL37 | |
| GGSGGGGSGGGGS (SEQ ID NO: 163) | NCL38 | |
| GHEAAAVMQVQYPAS (SEQ ID NO: | NCL39 | |
| 295) | | |
| GKGGSGKGGSGKGGS (SEQ ID NO: | NCL40 | |
| 296) | | |
| GKGKSGKGKSGKGKS (SEQ ID NO: | NCL41 | |
| 297) | | |
| GKGKSGKGKSGKGKSGKGKS (SEQ ID | NCL42 | |
| NO: 298) | | |
| GKPGSGKPGSGKPGS (SEQ ID NO: | NCL43 | |
| 299) | | |
| GKPGSGKPGSGKPGSGKPSGS (SEQ | NCL44 | |
| ID NO: 300) | | |
| GPAKELTPLKEAKVS (SEQ ID NO: 301) | NCL45 | |
| GSAGSAAGSGEF (SEQ ID NO: 302) | NCL46 | |
| GSGGG (SEQ ID NO: 166) | NCL47 | |
| GSGSG (SEQ ID NO: 167) | NCL48 | |
| GSS | NCL49 | |
| GSSG (SEQ ID NO: 168) | NCL50 | |
| GSSGGSGGSG (SEQ ID NO: 169) | NCL51 | |
| GSSGGSGGSGG (SEQ ID NO: 170) | NCL52 | |
| GSSGGSGGSGGS (SEQ ID NO: 171) | NCL53 | |
| GSSGGSGGSGGSG (SEQ ID NO: 172) | NCL54 | |
| GSSGGSGGSGGSGGGS (SEQ ID NO: | NCL55 | |
| 173) | | |
| GSSGGSGGSGS (SEQ ID NO: 174) | NCL56 | |
| GSSGT (SEQ ID NO: 175) | NCL57 | |
| GSSSG (SEQ ID NO: 176) | NCL58 | |
| GSTSGSGKPGSGEGSTKG (SEQ ID | NCL59 | |
| NO: 303) | | |
| GTAAAGAGAAGGAAAGAAG (SEQ ID | NCL60 | |
| NO: 304) | | |
| GTSGSSGSGSGGSGSGGGG (SEQ ID | NCL61 | |
| NO: 305) | | |
| IRPRAIGGSKPRVA (SEQ ID NO: 306) | NCL62 | |
| KESGSVSSEQLAQFRSLD (SEQ ID NO: | NCL63 | |
| 307) | | |
| KTTPKLEEGEFSEAR (SEQ ID NO: 308) | NCL64 | |
| PRGASKSGSASQTGSAPGS (SEQ ID | NCL65 | |
| NO: 309) | | |
| QPKAAP (SEQ ID NO: 310) | NCL66 | |
| QPKAAPSVTLFPP (SEQ ID NO: 311) | NCL67 | |
| RADAAAA(G4S)4 (SEQ ID NO: 312) | NCL68 | |
| RADAAAAGGPGS (SEQ ID NO: 313) | NCL69 | |
| RADAAP (SEQ ID NO: 314) | NCL70 | |
| RADAAPTVS (SEQ ID NO: 315) | NCL71 | |
| SAKTTP (SEQ ID NO: 316) | NCL72 | |
| SAKTTPKLEEGEFSEARV (SEQ ID NO: | NCL73 | |
| 317) | | |
| SAKTTPKLGG (SEQ ID NO: 318) | NCL74 | |
| STAGDTHLGGEDFD (SEQ ID NO: 319) | NCL75 | |
| TVAAP (SEQ ID NO: 320) | NCL76 | |
| TVAAPSVFIFPP (SEQ ID NO: 321) | NCL77 | |
| TVAAPSVFIFPPTVAAPSVFIFPP (SEQ | NCL78 | |
| ID NO: 322) | | |

In certain aspects, the MBM proprotein of the disclosure may comprise a polypeptide chain comprising, in an N- to C-terminal orientation, an ABS (or ABS chain), a hinge domain and a CH2 domain, and a CH3 domain. Thus, the hinge domain connects the ABS with the CH2 domain and can be said to constitute a type of linker. Exemplary hinge domains are set forth in Section 6.10.3.

### 6.6. TAA ABSs

The MBM proproteins of the disclosure comprise at least one ABS that binds specifically to an extracellular matrix (“ECM”) antigen, a tumor reactive lymphocyte antigen, a cell surface molecule of tumor or viral lymphocytes, a T-cell antigen (“TCA”), a checkpoint inhibitor, or a tumor-associated antigen (TAA), referred to herein as “TAA ABS”. The skilled artisan would recognize that the foregoing categories of target molecules are not mutually exclusive and thus a given target molecule may fall into more than one of the foregoing categories of target molecules. For example, some molecules may be considered both TAAs and ECM proteins, and other molecules may be considered both TCAs and checkpoint inhibitors. Preferably, the ECM antigen, tumor reactive lymphocyte antigen, cell surface molecule of tumor or viral lymphocytes, TCA, checkpoint inhibitor, or TAA is a human antigen. The antigen may or may not be present on normal cells. Particular aspects are directed to MBM proproteins comprising at least one ABS that binds specifically to a TAA. In certain embodiments, the TAA is preferentially expressed or upregulated on tumor cells as compared to normal cells. In other embodiments, the TAA is a lineage marker.

It is anticipated that any type of tumor and any type of ECM antigen, tumor reactive lymphocyte antigen, cell surface molecule of tumor or viral lymphocytes, TCA, checkpoint inhibitor, or TAA may be targeted by the MBM proproteins of the disclosure. Exemplary types of cancers that may be targeted include acute lymphoblastic leukemia, acute myelogenous leukemia, biliary cancer, B-cell leukemia, B-cell lymphoma, biliary cancer, bone cancer, brain cancer, breast cancer, triple-negative breast cancer, cervical cancer, Burkitt lymphoma, chronic lymphocytic leukemia, chronic myelogenous leukemia, colorectal cancer, endometrial cancer, esophageal cancer, gall bladder cancer, gastric cancer, gastrointestinal tract cancer, glioma, hairy cell leukemia, head and neck cancer, Hodgkin's lymphoma, liver cancer, lung cancer, medullary thyroid cancer, melanoma, multiple myeloma, ovarian cancer, non-Hodgkin's lymphoma, pancreatic cancer, prostate cancer, pulmonary tract cancer, renal cancer, sarcoma, skin cancer, testicular cancer, urothelial cancer, and other urinary bladder cancers. However, the skilled artisan will realize that TAAs and other target molecules associated with the tumor microenvironment are known for virtually any type of cancer.

Non-limiting examples of ECM antigens include syndecan, heparanase, integrins, osteopontin, link, cadherins, laminin, laminin type EGF, lectin, fibronectin, notch, nectin (e.g., nectin-4), tenascin, collagen (e.g., collagen type X) and matrixin.

Other target molecules are cell surface molecules of tumor or viral lymphocytes, for example T-cell co-stimulatory proteins such as CD27, CD28, 4-1BB (CD137), OX40, CD30, CD40, ICOS, lymphocyte function-associated antigen-1 (LFA-1), CD2, CD7, LIGHT, NKG2C, and B7-H3.

In particular embodiments, the target molecules are checkpoint inhibitors, for example CTLA-4, PD1, PDL1, PDL2, B7-H3, B7-H4, BTLA, HVEM, TIM3, GAL9, LAG3, VISTA, KIR, 2B4, CD160, CGEN-15049, CHK1, CHK2. In particular embodiments, the target molecule is PD1. In other embodiments, the target molecule is LAG3. In some embodiments, where the target molecule is a checkpoint inhibitor, the TAA ABS is non-blocking or poorly-blocking of ligand-receptor binding. Examples of non-blocking or poorly-blocking anti-PD1 antibodies includes antibodies having VH/VL amino acid sequences of SEQ ID Nos: 2/10 of PCT Pub. No. WO2015/112800A1; SEQ ID Nos: 16/17 of U.S. Pat. No. 11,034,765 B2; SEQ ID Nos. 164/178, 165/179, 166/180, 167/181, 168/182, 169/183, 170/184, 171/185, 172/186, 173/187, 174/188, 175/189, 176/190 and 177/190 of U.S. Pat. No. 10,294,299 B2. Examples of non-blocking or poorly-blocking anti-LAG3 antibodies includes antibodies having VH/VL amino acid sequences of SEQ ID Nos 23/24, 3/4 and 11/12 of US Pub. US2022/0056126A1.

In certain embodiments, the target molecules are TAAs. Exemplary TAAs are set forth in Table F below, together with references to exemplary antibodies or antibody 5 sequences upon which the TAA ABS can be based.

TABLE F
Exemplary Anti-TAAs

| Target | Antibody Name and/or Binding Sequences |
|---|---|
| 1-92-LFA-3 | Amevive ™ (alefacept) |
| 5T4 | GEN1044 |
| Activin Receptor Type II | Bimagrumab |
| | VH: SEQ ID NOs: 107, 109 of U.S. Pat. No. 8,388,968 B2 |
| | VL: SEQ ID NOs: 93, 95 of U.S. Pat. No. 8,388,968 B2 |
| B7-H3 | Obrindatamab (MGD009) |
| BAFF/B Lymphocyte | Benlysta ™ (velimumab) |
| Stimulator | |
| BAFF/B Lymphocyte | VH: amino acids 1-123 of SEQ ID NO: 327 of U.S. Pat. No. 7,138,501 |
| Stimulator | VL: amino acids 139-249 of SEQ ID NO: 327 of U.S. Pat. No. 7,138,501. |
| BAFF/B Lymphocyte | VH: amino acids 1-126 of SEQ ID NO: 1321 of U.S. Pat. No. 7,605,236; |
| Stimulator | VL: amino acids 143-251 of SEQ ID NO: 1049 of U.S. Pat. No. 7,605,236. |
| BAFF/B Lymphocyte | Belimumab |
| Stimulator | |
| BCMA | VH: the VH sequence of the heavy chain of SEQ ID NO. |
| | 126 of US 2021/0206865 A1 |
| | VL: the VL sequence of the light chain of SEQ ID NO. 129 |
| | or SEQ ID NO. 132 of US 2021/0206865 A1 |
| CA125 | Igobumab |
| CA125 | OvaRex ™ (oregobumab) |
| CD11a | Raptiva ™ (efalizumab) |
| | Sequence in Werther et al., 1996, The Journal of |
| | Immunology 157(11): 4986-4995. |
| CD19 | Blincyto ™ (blinatumomab) |
| CD19 | SGN-CD19A |
| CD20 | Bexxar ™ (tositumomab) |
| | VH: the VH sequence of the heavy chain of SEQ ID |
| | NO: 124 of US Patent Pub. US 2017/0002060 A1 |
| | VL: the VL sequence of the light chain of SEQ ID NO: 125 |
| | of US Patent Pub. US 2017/0002060 A1 |
| CD20 | Zevalin ™ (ibritumomab tiuxetan) |
| | VH: SEQ ID NO: 9 of U.S. Pat. No. 5,736,137 |
| | VL: SEQ ID NO: 6 of U.S. Pat. No. 5,736,137 |
| CD20 | Rituxan ™ (rituximab) |
| | VH: SEQ ID NO: 9 of U.S. Pat. No. 5,736,137 |
| | VL: SEQ ID NO: 6 of U.S. Pat. No. 5,736,137 |
| CD20 | Ocrevus ™ (ocrelizumab) |
| CD20 | Okaratuzumab |
| CD20 | Arzerra ™ (ofatumumab) |
| | VH: SEQ ID NO: 2 of U.S. Pat. No. 8,529,902 |
| | VL: SEQ ID NO: 4 of U.S. Pat. No. 8,529,902 |
| CD20 | Gazyva ™ (obinutuzumab) |
| CD20 | VH: SEQ ID NO: 4 of US 2021/0206870 A1 |
| | VL of SEQ ID NO: 6 of US 2021/0206870 A1 |
| CD20 | epcoritamab |
| CD22 | Belimumab |
| CD22 | Epratuzumab |
| CD22 | Besponsa ™ (inotuzumab ozogamicin) |
| CD22 | Lumoxiti ™ (moxetumumab pasudox) |
| CD22 | pinatuzumab vedotin |
| CD25 | Zenapax ™ (daclizumab) |
| | VH: SEQ ID NO: 9 of U.S. Pat. No. 7,060,269 |
| | VL: SEQ ID NO: 10 of U.S. Pat. No. 7,060,269 |
| CD30 | Adcetris ™ (brentuximab vedotin) |
| | VH: SEQ ID NO: 2 of U.S. Pat. No. 7,090,843 |
| | VL: SEQ ID NO: 10 of U.S. Pat. No. 7,090,843 |
| CD33 | Myelotarg ™ (gemtuzumab) |
| | Sequence in Man Sung, et al., 1993, Molecular |
| | immunology 30: 1361-1367 |
| CD33 | Lintuzumab |
| CD38 | Darzalex ™ (daratumumab) |
| CD40 | Lukatumumab |
| CD40 | Dacetuzumab |
| CD40L | Hu5c8 (ruplizumab) |
| CD44v6 | vibatuzumab mertansine |
| CD52 | Campath ™ (alemtuzumab) |
| | VH: SEQ ID NO: 1 of US Patent Pub. US 2017/0002060 A1 |
| | VL: SEQ ID NO: 2 of US Patent Pub. US 2017/0002060 A1 |
| CD70 | Blenrep ™ (borsetuzumab mafodotin) |
| CD123 | flotetuzumab |
| CD221 | Tepezza ™ (teprotumumab) |
| CEA | Hybri-Ceaker ® (altumomab pentetate) |
| CEA | Scintimun ™ (besilesomab) |
| CEA | CEA-CIDE ™ (labetuzumab)) |
| CEA | CEA-Scan ™ (arcitumomab) |
| CEA | hMN-15 |
| | CDR-H1, CDR-H2 and CDR-H3 sequences of SEQ ID |
| | NOs: 4-6 of U.S. Pat. No. 8,771,690 B2 |
| | CDR-L1, CDR-L2 and CDR-L3 sequences of SEQ ID |
| | NOs: 1-3 of U.S. Pat. No. 8,771,690 B2 |
| CEA | CEA binding portion of RO6958688/RG7802 from clinical |
| | trial NCT02324257 |
| CEA | Cibisatamab |
| CEA | CEA binding portion of MEDI-565/MT110/AMG211 from |
| | clinical trials NCT01284231 and NCT02291614 |
| | VH: SEQ ID NO: 49 or 51 of PCT Publication No. WO |
| | 2013/012414 A1 |
| | VL: SEQ ID NO: 48 of PCT Publication No. WO |
| | 2013/012414 A1. |
| CEA | Rabetuzumab |
| CEA | Atezolizumab |
| CEA | Cibisatamab |
| CEA | MEDI-565 (AMG211, MT111) |
| CEA | RO6958688 |
| CEA | VH: SEQ ID No. 9 described in WO2022/048883A1 |
| | VL: SEQ ID No. 10 described in WO2022/048883A1 |
| CLDN18.2 | AMG910 |
| Complement C5 | Soliris ™ (eculizumab) |
| | VH: amino acids 1-122 of SEQ ID NO: 10 of US Patent |
| | No. 6,355,245 |
| | VL: amino acids 3-110 of SEQ ID NO: 9 of US Patent No. |
| | 6,355,245 |
| CTLA-4 | Yervoy ™ (ipilimumab) |
| | VH: SEQ ID NO: 17 of WO 2001/014424 A2 |
| | VL: SEQ ID NO: 7 of WO 2001/014424 A2 |
| CTLA-4 | (tremelimumab) |
| CTLA-4 | Orencia ™ (abatacept) |
| DLL3 | AMG757 |
| EGFR | Erbitux ™ (cetuximab) |
| | VH: SEQ ID NO: 11 of U.S. Pat. No. 6,217,866 |
| | VL: SEQ ID NO: 13 of U.S. Pat. No. 6,217,866 |
| EGFR | Vectibix ™ (panitumumab) |
| | VH: SEQ ID NO: 37 of U.S. Pat. No. 6,235,883 |
| | VL: SEQ ID NO: 38 of U.S. Pat. No. 6,235,883 |
| EGFR | Zalutumumab |
| | VH: SEQ ID NO: 64 of WO 2018/140831 A2 |
| | VL: SEQ ID NO: 69 of WO 2018/140831 A2 |
| EGFR | mapatumumab |
| EGFR | Matuzumab |
| EGFR | Nimotuzumab |
| | VH: SEQ ID NO: 51 of WO 2018/140831 A2 |
| | VL: SEQ ID NO: 56 of WO 2018/140831 A2 |
| EGFR | ICR62 |
| EGFR | mAb 528 |
| EGFR | CH806 |
| EGFRv3 | AMG596 |
| EGFRv3 | AMG404 |
| EGFR/CD64 | MDX-447 |
| EpCAM | Panorex ™ (edrecolomab) |
| | VH: SEQ ID NO: 129 of WO 2018/140831 A2 |
| | VL: SEQ ID NO: 134 of WO 2018/140831 A2 |
| EpCAM | Adecatumumab |
| | VH: SEQ ID NO: 142 of WO 2018/140831 A2 |
| | VL: SEQ ID NO: 147 of WO 2018/140831 A2 |
| EpCAM | tucotuzumab celmoleukin |
| EpCAM | citatuzumab bogatox |
| EpCAM | EP1629013 B1 |
| | VH: SEQ ID NOs: 80, 84, 88, 92 or 96 |
| | VL: SEQ ID NOs: 82, 86, 90, 94 or 98 |
| EpCAM | G8.8 |
| | HC: SEQ ID NO: 4 of US Patent Pub. No. US |
| | 2020/0317806 A1 |
| | HL: SEQ ID NO: 3 of US Patent Pub. No. US |
| | 2020/0317806 A1 |
| EpCAM | VH: SEQ ID NOs: 17-22 of WO 2021/211510 A2. |
| | VL: SEQ ID NO: 15-16 of WO 2021/211510 A2. |
| EpCAM | Removab ™(catumaxomab) |
| EpCAM | Vicineum ™ (oportuzumab monatox) |
| EpCAM | M701 |
| F protein of RSV | Synagic ™ (palivizumab) |
| GD2 | 3F8 |
| Glycoprotein receptor IIb/IIIa | ReoPro ™ (abiciximab) |
| gpA33 | MGD007 |
| GPC3 | ERY974 |
| GUCY2C | PF-07062119 |
| Her2 | Herceptin ™ (trastuzumab) |
| Her2 | Aldesleukin (proleukine) |
| Her2 | Sargramustim (leukine) |
| Her2 | M802 |
| Her2 | Runimotamab (BTRC4017A, R07227780) |
| Her2 | ISB1302 |
| Her2-neu | Perjeta ™ (pertuzumab) |
| | VH: SEQ ID NO: 16 of WO 2013/096812 A1. |
| | VL: SEQ ID NO: 15 of WO 2013/096812 A1. |
| Her2-neu | Rexomun ™ (ertumaxomab) |
| IgE | Xolair ™ (omalizumab) |
| IGFIR | (figitumumab) |
| IL1β | Ilaris ™ (canakinumab) |
| | VH: SEQ ID NO: 1 of U.S. Pat. No. 7,446,175. |
| | VL: SEQ ID NO: 2 of U.S. Pat. No. 7,446,175 |
| IL12/IL23 | Stelara ™ (ustekinumab) |
| IL1Ra | Antril ™, Kineret ™ (ankinra) |
| IL2R | Simulect ™ (basiliximab) |
| | VH: SEQ ID NO: 3 of U.S. Pat. No. 6,383,487 |
| | VL: SEQ ID NO: 6 of U.S. Pat. No. 6,383,487 |
| IL6 | Clazakizumab |
| IL6 receptor | Actemra ™ (tocilizumab) |
| | VH: SEQ ID NO: 31 of U.S. Pat. No. 7,479,543 |
| | VL: SEQ ID NO: 29 of U.S. Pat. No. 7,479,543 |
| IL12/IL23 p40 subunit | Stelara ™ (ustekinumab) |
| | VH: SEQ ID NO: 7 of U.S. Pat. No. 6,902,734 |
| | VL: SEQ ID NO: 8 of U.S. Pat. No. 6,902,734 |
| Integrinα4 | Tysabri ™ (natalizumab) |
| | VH: SEQ ID NOs: 11-13 of U.S. Pat. No. 5,840,299 |
| | VL: SEQ ID NOs: 7-8 of U.S. Pat. No. 5,840,299 |
| Integrinα4 β7 | Entyvio ™ (vedolizumab) |
| | HC: SEQ ID NO: 2 of US Patent Pub. US 2012/0282249 |
| | LC: SEQ ID NO: 4 of US Patent Pub. US 2012/0282249. |
| Mesothelin | Amatuximab |
| Mesothelin | HPN536 |
| MUC1 | civatuzumab tetraxetane |
| MUC1 | Pankomab ™ (gatipotuzumab) |
| MUC1 | femtumumab |
| MUC1 | Cantuzumab ravtansine |
| MUC 16 (CA125) | Anti-MUC16 antibodies having VH and VL sequences |
| | having the amino acid sequences of any one of the |
| | following SEQ ID NO: pairs from US 2018/0118848A1: |
| | 18/26; 82/858; 98/170 |
| MUC17 | AMG199 |
| NGF | (tanezumab) |
| Phosphatidylserine | (bavituximab) |
| PSCA | GEM3PSCA |
| PSMA | huJ591 |
| PSMA | Anti-PSMA antibodies having VH and VL sequences |
| | having the amino acid sequences of any one of the |
| | following SEQ ID NO: pairs from WO 2017/023761A1: |
| | 2/1642; 10/1642; 18/1642; 26/1642; 34/1642; 42/1642; |
| | 50/1642; 58/1642; 66/1642; 74/1642; 82/1642; 90/1642; |
| | 98/1642; 106/1642; 1 14/1642; 122/130; and 138/146. |
| PSMA | An antibody such as: PSMA 3.7, PSMA 3.8, PSMA 3.9, |
| | PSMA 3.11, PSMA 5.4, PSMA 7.1, PSMA 7.3, PSMA |
| | 10.3, PSMA 1.8.3, PSMA A3.1.3, PSMA A3.3.1, Abgenix |
| | 4.248.2, Abgenix 4.360.3, Abgenix 4.7.1, Abgenix 4.4.1, |
| | Abgenix 4.177.3, Abgenix 4.16.1, Abgenix 4.22.3, Abgenix |
| | 4.28.3, Abgenix 4.40.2, Abgenix 4.48.3, Abgenix 4.49.1, |
| | Abgenix 4.209.3, Abgemx 4.219.3, Abgenix 4.288.1, |
| | Abgenix 4.333.1, Abgemx 4.54.1, Abgenix 4.153.1, |
| | Abgenix 4.232.3, Abgenix 4.292.3, Abgenix 4.304.1, |
| | Abgenix 4.78.1 and Abgenix 4.152.1 described in |
| | WO2003034903A2 |
| | A hybridoma cell line such as: PSMA 3.7 (PTA-3257), |
| | PSMA 3.8, PSMA 3.9 (PTA- 3258), PSMA 3.11 (PTA- |
| | 3269), PSMA 5.4 (PTA-3268), PSMA 7.1 (PTA-3292), |
| | PSMA 7.3 (PTA-3293), PSMA 10.3 (PTA-3247) , PSMA |
| | 1.8.3 (PTA-3906), PSMA A3.1.3 (PTA- 3904), PSMA |
| | A3.3.1 (PTA-3905), Abgenix 4.248.2 (PTA-4427), Abgenix |
| | 4.360.3 (PTA- 4428), Abgenix 4.7.1 (PTA-4429), Abgenix |
| | 4.4.1 (PTA-4556), Abgenix 4.177.3 (PTA-4557), Abgenix |
| | 4.16.1 (PTA-4357), Abgenix 4.22.3 (PTA-4358), Abgenix |
| | 4.28.3 (PTA-4359), Abgenix 4.40.2 (PTA-4360), Abgenix |
| | 4.48.3 (PTA-4361), Abgenix 4.49.1 (PTA-4362), Abgenix |
| | 4.209.3 (PTA-4365), Abgenix 4.219.3 (PTA-4366), |
| | Abgenix 4.288.1 (PTA-4367), Abgenix 4.333.1 (PTA- |
| | 4368), Abgenix 4.54.1 (PTA-4363), Abgenix 4.153.1 (PTA- |
| | 4388), Abgenix 4.232.3 (PTA-4389), Abgenix 4.292.3 |
| | (PTA-4390), Abgenix 4.304.1 (PTA-4391), Abgenix 4.78.1 |
| | (PTA-4652), and Abgemx 4.152.1(PTA-4653) described in |
| | WO 2003/034903A2. |
| | VH of SEQ ID NOs: 2-7 described in WO 2003/034903A2 |
| | VL of SEQ ID NOs: 8-13 described in WO 2003/034903A2 |
| PMSA | VH: SEQ ID NOs: 225, 239, 253, 267, 281, 295, 309, |
| | 323, 337, 351, 365, 379, 393, 407, 421, 435, 449, 463, |
| | 477, 491, 505, 519, 533, 547, 561, 575, 589, 603 or 617 |
| | described in WO 2011/121110A1. |
| | VL SEQ ID NOs: 230, 244, 258, 272, 286, 300, 314, 328, |
| | 342, 356, 370, 384, 398, 412, 426, 440, 454, 468, 482, |
| | 496, 510, 524, 538, 552, 566, 580, 594, 608 or 622 |
| | described in WO 2011/121110A1. |
| | VH and VL SEQ ID Nos: 235, 249, 263, 277, 291, 305, |
| | 319, 333, 347, 361, 375, 389, 403, 417, 431, 445, 459, |
| | 473, 487, 501, 515, 529, 543, 557, 571, 585, 599, 613 or |
| | 627 described in WO 2011/121110A1. |
| PMSA | An anti-PMSA antibody having a VL amino acid sequence |
| | of any one of SEQ ID NOs: 229-312 of US 2022/0119525 |
| | A1 and a VH of SEQ ID NO: 217 of US 2022/0119525 A1. |
| PMSA | ES414 |
| PMSA | BAY2010112 (pasotuxizumab) |
| PMSA | CCW702 |
| PMSA | JNJ-63898081 |
| PMSA | CC-1 |
| PMSA | Acapatamab |
| PSMA | HPN424 |
| RAAG12 | RAV12 |
| RANKL | Prolia ™ (denosumab) |
| | VH: SEQ ID NO: 51 of US Patent Pub. 2017/0002060 |
| | VL: SEQ ID NO: 52 of US Patent Pub. 2017/0002060 |
| SLAMF7 | Empliciti ™ (elotuzumab) |
| SSTR2 | XmAb ®18087 |
| STEAP1 | VHCDR1 SEQ ID NOs: 14, 33, 182, 184 or 185 described |
| | in US20210179731A1. |
| | VHCDR2 SEQ ID NOs: 15, 21, 34, 182, 184 or 185 |
| | described in US20210179731A1. |
| | VHCDR3 SEQ ID NOs: 16 and 35 described in |
| | US20210179731A1. |
| | VH SEQ ID NOs: 182 or 184 described in |
| | US20210179731A1. |
| | VLCDR1 SEQ ID NOs: 11 or 30 described in |
| | US20210179731A1 |
| | VLCDR2 SEQ ID NOs: 12 or 31 described in |
| | US20210179731A1. |
| | VLCDR3 SEQ ID NOs: 13 or 32 described in |
| | US20210179731A1. |
| | VL SEQ ID NOs: 183 or 186 described in |
| | US20210179731A1. |
| STEAP1 | AMG509 |
| STEAP2 | Anti-STEAP 2 antibodies having CDR-H1, CDR-H2, CDR- |
| | H3, CDR-L1, CDR-L2 and CDR-L3 sequences selected |
| | from SEQ ID NOS: (1) 4-6-8-12-14-16; (2) 20-22-24-28- |
| | 30-32; (3) 36-38-40-44-46-48; (4) 52-54-56-60-62-64; (5) |
| | 68-70-72-60-62-64; (6) 76-78-80-60-62-64; (7) 84-86-88- |
| | 60-62-64; (8) 92-94-96-60-62-64; (9) 100-102-104-60-62- |
| | 64; (10) 108-110-112-116-118-120; (11) 124-126-128- |
| | 132-134-136; (12) 140-142-144-148-150-152; (13) 156- |
| | 158-160-164-166-168; (14) 172-174-176-180-182-184; |
| | (15) 188-190-192-196-198-200; (16) 204-206-208-212- |
| | 214-216; (17) 220-222-224-228-230-232; (18) 236-238- |
| | 240-244-246-248; (19) 252-254-256-260-262-264; (20) |
| | 268-270-272-276-278-280; (21) 284-286-288-292-294- |
| | 296; (22) 300-302-304-308-310-312; (23) 316-318-320- |
| | 324-326-328; (24) 332-334-336-340-342-344; (25) 348- |
| | 350-352-356-358-360; (26) 364-366-368-372-374-376; |
| | and (27) 380-382-384-388-390-392 of U.S. Pat. No. |
| | 10,772,972 B2. |
| | Anti-STEAP 2 antibodies having (a) a VH comprising the |
| | amino acid of any one of SEQ ID NOs: 2, 18, 34, 50, 66, |
| | 74, 82, 90, 98, 106, 122, 138, 154, 170, 186, 202, 218, |
| | 234, 250, 266, 282, 298, 314, 330, 346, 362, and 378 of |
| | U.S. Pat. No. 10,772,972 B2; and (b) a VL comprising |
| | the amino acid sequence of any one of SEQ ID NOs: 10; |
| | 26; 42; 58; 114; 130; 146; 162; 178; 194; 210; 226, 242; |
| | 258; 274; 290; 306; 322; 338; 354; 370; and 386 of U.S. |
| | Pat. No. 10,772,972 B2. |
| | Anti-STEAP 2 antibodies having a VH/VL pair comprising |
| | the amino acid sequences of any of the following pairs of |
| | SEQ ID NOs of U.S. Pat. No. 10,772,972 B2: 2/10; |
| | 18/26; 34/42; 50/58; 66/58; 74/58; 82/58; 90/58; 98/58; |
| | 106/114; 122/130; 138/146; 154/162; 170/178; 186/194; |
| | 202/210; 218/226; 234/242; 250/258; 266/274; 282/290; |
| | 298/306; 314/322; 330/338; 346/354; 362/370; and |
| | 378/386. |
| TGFβ | GC1008 |
| TNFR | Enbrel ™ (etanercept) |
| TNFα | Remicade ™ (infliximab) |
| | VH: SEQ ID NO: 2 of Int. Patent Publication |
| | WO201/3087911 A1 |
| | VH: SEQ ID NO: 3 of Int. Patent Publication WO2013/ |
| | A1087911 |
| TNFα | Humira ™ (adalimumab) |
| | VH: SEQ ID NO: 4 of U.S. Pat. No. 6,258,562 |
| | VL: SEQ ID NO: 3 of U.S. Pat. No. 6,258,562 |
| TNFα | Cimzia ™ (certolizumab pegol) |
| | VH: SEQ ID NO: 14 of U.S. Pat. No. 7,012,135 |
| | VL: SEQ ID NO: 9 of U.S. Pat. No. 7,012,135 |
| TNFα | Simponi ™ (golimumab) |
| | VH: SEQ ID NO: 7 of U.S. Pat. No. 7,250,165 |
| | VL: SEQ ID NO: 8 of U.S. Pat. No. 7,250,165 |
| VEGF | Avastin ™ (bevacizumab) |
| | VH: SEQ ID NO: 9 of U.S. Pat. No. 7,060,269 |
| | VL: SEQ ID NO: 10 of U.S. Pat. No. 7,060,269 |
| VEGF | Lucentis ™ (ranibizumab) |
| | VH: SEQ ID NO: 4 of U.S. Pat. No. 9,914,770 |
| | VL: SEQ ID NO: 2 of U.S. Pat. No. 9,914,770 |

In some aspects, the TAA ABS competes with an antibody set forth in Table F for binding to the TAA. In further aspects, the TAA ABS comprises CDRs having CDR sequences of an anti-TAA antibody set forth in Table F. In some embodiments, the TAA ABS comprises all 6 CDR sequences of the anti-TAA antibody set forth in Table F. In other embodiments, the TAA ABS comprises at least the heavy chain CDR sequences (CDR-H1, CDR-H2, CDR-H3 and the light chain CDR sequences of a universal light chain. In further aspects, a TAA ABS comprises a VH comprising the amino acid sequence of the VH of an anti-TAA antibody set forth in Table F. In some embodiments, the TAA ABS further comprises a VL comprising the amino acid sequence of the VL of the anti-TAA antibody set forth in Table F. In other embodiments, the TAA ABS further comprises a universal light chain VL sequence.

Additional TAAs that can be targeted by the MBM proproteins are disclosed in Table K below and in, e.g., Hafeez et al., 2020, Molecules 25:4764, doi:10.3390/molecules25204764, particularly in Table 1. Table 1 of Hafeez et al. is incorporated by reference in its entirety here.

Yet additional exemplary TAAs include Fibroblast Activation Protein (FAP), the A1 domain of Tenascin-C (TNC A1), the A2 domain of Tenascin-C (TNC A2), the Extra Domain B of Fibronectin (EDB), the Melanoma-associated Chondroitin Sulfate Proteoglycan (MCSP), MART-1/Melan-A, gp100, Dipeptidyl peptidase IV (DPPIV), adenosine deaminase-binding protein (ADAbp), cyclophilin b, colorectal associated antigen (CRC)-C017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, c-erbB-2, Her2, EGFR, IGF-1R, CD2 (T-cell surface antigen), CD3 (heteromultimer associated with the TCR), CD22 (B-cell receptor), CD23 (low affinity IgE receptor), CD30 (cytokine receptor), CD33 (myeloid cell surface antigen), CD40 (tumor necrosis factor receptor), IL-6R-(IL6 receptor), CD20, MCSP, PDGFβR (β-platelet-derived growth factor receptor), ErbB2 epithelial cell adhesion molecule (EpCAM), EGFR variant III (EGFRvIII), CD19, disialoganglioside GD2, ductal-epithelial mucine, gp36, TAG-72, glioma-associated antigen, β-human chorionic gonadotropin, alphafetoprotein (AFP), lectin-reactive AFP, thyroglobulin, MN-CA IX, human telomerase reverse transcriptase, RU1, RU2 (AS), intestinal carboxyl esterase, mut hsp70-2, M-CSF, prostase, prostase specific antigen (PSA), PAP, LAGA-1a, p53, prostein, PSMA, surviving and telomerase, prostate-carcinoma tumor antigen-1 (PCTA-1), ELF2M, neutrophil elastase, ephrin B2, insulin growth factor (IGF1)-I, IGF-II, IGFI receptor, 5T4, ROR1, Nkp30, NKG2D, tumor stromal antigens, the extra domain A (EDA) and extra domain B (EDB) of fibronectin and the A1 domain of tenascin-C (TnC A1).

### 6.7. TCE ABSs

The MBM proproteins of the disclosure comprise at least a T-cell engaging ABS that binds specifically to a T-cell receptor complex component, referred to herein as “TCE ABS”. Exemplary targets for the TCE ABS are CD3 and the T-cell receptor (e.g., TCRαβ or TCRγδ). Preferably, the TCE ABS target is a human T-cell receptor complex component. The epitope of the TCE ABS can be an individual polypeptide (e.g., CD3 epsilon) or a multimeric component of the T-cell receptor complex (e.g., the TCRαβ dimer or the TCRγδ dimer).

Exemplary CD3 and TCR antibodies or antibody sequences are set forth in Table G below, upon which the TAA ABS can be based.

TABLE G
Exemplary T-Cell Engaging Antibodies

| Target | Antibody Name and/or Binding Sequences |
|---|---|
| CD3 | Catumaxomab |
| CD3 | (ertumaxomab) |
| CD3 | The CD3 portion of anti-PSMA/anti-CD3 antibodies described in WO2011121110A1 |
| CD3 | Anti-CD3 antibody sequences in US10266593B2 |
| CD3 | Anti-CD3 antibody sequences in U.S. Pat. No. 8846042B2 |
| CD3 | Anti-CD3 antibody sequences US 2016/0355600 |
| CD3 | Anti-CD3 antibody sequences in WO 2014/110601 |
| CD3 | Anti-CD3 antibody sequences in WO 2014/145806 |
| CD3 | Anti-CD3 antibody sequences in U.S. Pat. No. 10,066,015 |
| CD3 | Anti-CD3 antibody sequences in WO 2019/034580 |
| CD3 | Anti-CD3 antibody sequences in WO 2014/056783 |
| CD3 | Anti-CD3 antibody sequences in WO 2013/055809 A1 |
| CD3 | Anti-CD3 antibody sequences in US 10,066,016 |
| CD3 | Anti-CD3 antibody sequences in US 2010/0150918 |
| CD3 | MT110 |
| CD3 | Acapatamab (AMG160) |
| CD3 | AMG199 |
| CD3 | AMG330 |
| CD3 | AMG427 |
| CD3 | AMG562 |
| CD3 | AMG596 |
| CD3 | AMG673 |
| CD3 | AMG701 |
| CD3 | Tarlatamab (AMG757) |
| CD3 | AMG910 |
| CD3 | BAY2010112 |
| CD3 | AMG420 |
| CD3 | AMG424 |
| CD3 | AMG509 |
| CD3 | AMV564 |
| CD3 | APVO436 |
| CD3 | Alnuctamab (CC-93269) |
| CD3 | ERY974 |
| CD3 | A-319 |
| CD3 | GEM333 |
| CD3 | GEM3PSCA |
| CD3 | Cevostamab |
| CD3 | Runimotamab |
| CD3 | GEN1044 |
| CD3 | Epcoritamab |
| CD3 | HPN424 |
| CD3 | ISB1302 |
| CD3 | ISB1342 |
| CD3 | IGM-2323 |
| CD3 | IMC-F106C |
| CD3 | IMC-C103C |
| CD3 | IMCnyeso |
| CD3 | JNJ-63709178 |
| CD3 | JNJ-63898081 |
| CD3 | Teclistamab |
| CD3 | Talquetamab |
| CD3 | JNJ-67571244 |
| CD3 | MGD007 |
| CD3 | Orlotamab |
| CD3 | Duvortuxizumab |
| CD3 | Flotetuzumab |
| CD3 | MCLA-117 |
| CD3 | PF-06671008 |
| CD3 | Elranatamab |
| CD3 | Odronextamab |
| CD3 | REGN5458 |
| CD3 | REGN5459 |
| CD3 | REGN4018 |
| CD3 | Glofitamab |
| CD3 | RO6958688 |
| CD3 | SAR440234 |
| CD3 | TNB-383B |
| CD3 | M802 |
| CD3 | Xmab13676 |
| CD3 | Xmab18087 |
| CD3 | Vibecotamab |
| CD3 | Nivatrotamab |
| CD3 | Anti-CD3 antibody sequences in US20190211100 |
| CD3 | Anti-CD3 antibody sequences in EP1629011B |
| CD3 | VH of SEQ ID NOS. 90 and 98 disclosed in US 2021/0206865 A1 |
| | CDR-H1 of SEQ ID NO: 92 and 100 disclosed in US 2021/0206865 A1 |
| | CDR-H2 of SEQ ID NO: 94 and 102 disclosed in US 2021/0206865 A1 |
| | CDR-H3 of SEQ ID NO: 96 and 104 disclosed in US 2021/0206865 A1 |
| | HC of SEQ ID NO: 127 or SEQ ID NO: 128 disclosed in US 2021/0206865 A1 |
| | LC of SEQ ID NO: 129 or SEQ ID NO: 132 disclosed in US 2021/0206865 A1 |
| CD3 | Anti-CD3 Heavy chain of SEQ ID NO: 2 disclosed in US 2021/0206870 A1 |
| | Anti-CD3 VH SEQ ID NO: 5 disclosed in US 2021/0206870 A1 |
| | Anti-CD3 VL of SEQ ID NO: 6 disclosed in US 2021/0206870 A1 |
| | Anti-CD3 CDR-H1 of SEQ ID NO: 10 disclosed in US 2021/0206870 A1 |
| | Anti-CD3 CDR-H2 of SEQ ID NO: 11 disclosed in US 2021/0206870 A1 |
| | Anti-CD3 CDR-H3 of SEQ ID NO: 12 disclosed in US 2021/0206870 A1 |
| CD3 | Anti-CD3 VH of SEQ ID NO: 92, 102, 112, 122, 132, 142, 156, 166, 176, 186, 196 or |
| | 206 disclosed in US 2022/0119525 A1 |
| | Anti-CD3 CDR-H1 of SEQ ID NO: 93, 103, 113, 123, 133, 143, 157, 167, 177, 187, |
| | 197 or 207 disclosed in US 2022/0119525 A1 |
| | Anti-CD3 CDR-H2 of SEQ ID NO: 94, 104, 114, 124, 134, 144, 158, 168, 178, 188, |
| | 198 or 208 disclosed in US 2022/0119525 A1 |
| | Anti-CD3 CDR-H3 of SEQ ID NO: 95, 105, 115, 125, 135, 145, 159, 169, 179, 189, |
| | 199 or 209 disclosed in US 2022/0119525 A1 |
| | Anti-CD3 VL of SEQ ID NO: 96, 106, 116, 126, 136, 146, 152, 162, 172, 182, 192 or |
| | 202 disclosed in US 2022/0119525 A1 |
| | Anti-CD3 CDR-L1 of SEQ ID NO: 97, 107, 117, 127, 137, 147, 153, 163, 173, 183, |
| | 193 or 203 disclosed in US 2022/0119525 A1 |
| | Anti-CD3 CDR-L2 of SEQ ID NO. 98, 108, 118, 128, 138, 148, 154, 164, 174, 184, |
| | 194 or 204 disclosed in US 2022/0119525 A1 |
| | Anti-CD3 CDR-L3 of SEQ ID NO. 99, 109, 119, 129, 139, 149, 155, 165, 175, 185, |
| | 195 or 205 disclosed in US 2022/0119525 A1 |
| TCRαβ | BMA031 sequences disclosed in US 2012/0034221 |
| TCRγδ | 6TCS1 antibody disclosed in U.S. Pat. No. 5,980,892 |

In some aspects, the TCE ABS competes with a T-cell engaging (TCE) antibody set forth in Table G for binding to the TCE antibody's target (e.g., CD3 or a T-cell receptor). In further aspects, the TCE ABS comprises CDRs having CDR sequences of a TCE antibody set forth in Table G. In some embodiments, the TCE ABS comprises all 6 CDR sequences of the TCE antibody set forth in Table G. In other embodiments, the TCE ABS comprises at least the heavy chain CDR sequences (CDR-H1, CDR-H2, CDR-H3) and the light chain CDR sequences of a universal light chain. In further aspects, a TCE ABS comprises a VH comprising the amino acid sequence of the VH of a TCE antibody set forth in Table G. In some embodiments, the TCE ABS further comprises a VL comprising the amino acid sequence of the VL of the TCE antibody set forth in Table G. In other embodiments, the TCE ABS further comprises a universal light chain VL sequence.

### 6.8. Anti-TCE ABSs

In certain embodiments, the MBM proproteins of the disclosure comprise an anti-TCE

ABS. An anti-TCE ABS is a fragment of an anti-idiotypic antibody that is capable of specifically binding the TCE-ABS. Various types of antibody fragments may be used in the context of an anti-TCE ABS of the present disclosure including, for example, an scFv, a Fab, a VHH, and a VH. In some embodiments, the anti-TCE ABS is an scFv of an anti-idiotypic antibody. In some embodiments, the anti-TCE ABS is a VHH derived from an anti-idiotypic antibody. In some embodiments, the anti-TCE ABS is a VH derived from an anti-idiotypic antibody.

An anti-TCE ABS can be derived from a single domain antibody composed of a single VH or VL domain which exhibits sufficient affinity to the target. In a specific embodiment, the single domain antibody is a camelid VHH domain (see, e.g., Riechmann, 1999, Journal of Immunological Methods 231:25-38; WO 94/04678).

Anti-idiotypic antibodies are antibodies directed against the antigen-combining region or variable region (called the idiotype or Id) of another antibody molecule. In principle, immunization with an antibody molecule expressing a paratope (antigen-combining site) for a given antigen should produce a group of anti-antibodies, some of which share with the antigen a complementary structure to the paratope.

Exemplary methods of producing anti-idiotypic antibodies U.S. Pat. No. 10,150,817 B2 and in Example 1 of WO 2017/162587 A1.

Exemplary anti-CD3 anti-idiotypic ABSs that can be used as the anti-TCE ABSs of the disclosure are the scFvs designated as 4.15.64 and 4.32.63, which are anti-idiotypes of an anti-CD3 ABS designated as CH2527. The sequences of 4.15.64, 4.32.63 and CH2527 are disclosed in WO 2017/162587 A1.

### 6.9. ABS Formats

In certain aspects, an ABS in an MBM proprotein of the disclosure can be any type of antibody or fragment thereof that retains specific binding to an antigenic determinant. In one embodiment the ABS is an immunoglobulin molecule or fragment thereof, particularly an IgG class immunoglobulin molecule, more particularly an IgG1 or IgG4 immunoglobulin molecule. Antibody fragments include, but are not limited to, VH (or VH) fragments, VL (or VL) fragments, Fab fragments, F(ab′)₂ fragments, scFv fragments, Fv fragments, minibodies, diabodies, triabodies, and tetrabodies.

### 6.9.1. Fab

Fab domains were traditionally produced by proteolytic cleavage of immunoglobulin molecules using enzymes such as papain. The Fab domains can comprise constant domain and variable region sequences from any suitable species, and thus can be murine, chimeric, human or humanized.

Fab domains typically comprise a CH1 domain attached to a VH domain which pairs with a CL domain attached to a VL domain. In a wild-type immunoglobulin, the VH domain is paired with the VL domain to constitute the Fv region, and the CH1 domain is paired with the CL domain to further stabilize the binding site. A disulfide bond between the two constant domains can further stabilize the Fab domain.

For the MBM proproteins of the disclosure, particularly when the light chains of the ABSs are not common or universal light chains, it is advantageous to use Fab heterodimerization strategies to permit the correct association of Fab domains belonging to the same ABS and minimize aberrant pairing of Fab domains belonging to different ABSs. For example, the Fab heterodimerization strategies shown in Table H below can be used:

TABLE H
Fab Heterodimerization Strategies

| STRATEGY | VH | CH1 | VL | CL | REFERENCE |
|---|---|---|---|---|---|
| CrossMabCH1-CL | WT | CL | WT | CH1 | Schaefer et al., 2011, |
| | | domain | | domain | Cancer Cell 2011; |
| | | | | | 20: 472-86; |
| | | | | | PMID: 22014573. |
| orthogonal Fab | 39K, | H172A, | 1R, | L135Y, | Lewis et al., 2014, Nat |
| VHVRD1CH1CRD2 - | 62E | F174G | 38D, | S176W | Biotechnol 32: 191-8 |
| VLVRD1CλCRD2 | | | (36F) | | |
| orthogonal Fab | 39Y | WT | 38R | WT | Lewis et al., 2014, Nat |
| VHVRD2CH1wt - | | | | | Biotechnol 32: 191-8 |
| VLVRD2Cλwt | | | | | |
| TCR CαCβ | 39K | TCR Cα | 38D | TCR Cβ | Wu et al., 2015, MAbs |
| | | | | | 7: 364-76 |
| CR3 | WT | T192E | WT | N137K, | Golay at al., 2016, J |
| | | | | S114A | Immunol 196: 3199-211. |
| MUT4 | WT | L143Q, | WT | V133T, | Golay at al., 2016, J |
| | | S188V | | S176V | Immunol 196: 3199-211. |
| DuetMab | WT | F126C | WT | S121C | Mazor et al., 2015, MAbs |
| | | | | | 7: 377-89; Mazor et al., |
| | | | | | 2015, MAbs 7: 461-669. |
| Domain | WT | CH3 + knob or | WT | CH3 + | Wozniak-Knopp et al., |
| exchanged | | hole mutation | | hole or | 2018, |
| | | | | knob | PLoSONE13(4): e0195442 |
| | | | | mutation | |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH:VL and CH1:CL interface such that the Fab components preferentially pair with each other rather than with components of other Fabs.

In one embodiment, the one or more amino acid modifications are limited to the conserved framework residues of the variable (VH, VL) and constant (CH1, CL) domains as indicated by the Kabat numbering of residues. Almagro, 2008, Frontiers In Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions or a combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179, the contents of which are hereby incorporated by reference.

In some embodiments, the Fab domain comprises a 192E substitution in the CH1 domain and 114A and 137K substitutions in the CL domain, which introduces a salt-bridge between the CH1 and CL domains (see, e.g., Golay et al., 2016, J Immunol 196:3199-211).

In some embodiments, the Fab domain comprises a 143Q and 188V substitutions in the CH1 domain and 113T and 176V substitutions in the CL domain, which serves to swap hydrophobic and polar regions of contact between the CH1 and CL domain (see, e.g., Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014 Nature Biotechnology 32:191-198). In an embodiment, 39K, 62E modifications are introduced in the VH domain, H172A, F174G modifications are introduced in the CH1 domain, 1 R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1:CL disulfide bond with an engineered disulfide bond, thereby increasing the efficiency of Fab component pairing. For example, an engineered disulfide bond can be introduced by introducing a 126C in the CH1 domain and a 121 C in the CL domain (see, e.g., Mazor et al., 2015, MAbs 7:377-89).

Fab domains can also be modified by replacing the CH1 domain and CL domain with alternative domains that promote correct assembly. For example, Wu et al., 2015, MAbs 7:364-76, describes substituting the CH1 domain with the constant domain of the T cell receptor and substituting the CL domain with the b domain of the T cell receptor, and pairing these domain replacements with an additional charge-charge interaction between the VL and VH domains by introducing a 38D modification in the VL domain and a 39K modification in the VH domain.

In lieu of, or in addition to, the use of Fab heterodimerization strategies to promote correct VH-VL pairings, the VL of common light chain (also referred to as a universal light chain) can be used for each unique ABS in the MBM proproteins of the disclosure. In various embodiments, employing a common light chain as described herein reduces the number of inappropriate species in the MBM proproteins as compared to employing original cognate VLs. In various embodiments, the VL domains of ABSs are identified from monospecific antibodies comprising a common light chain. In various embodiments, the VH regions of the ABSs in the MBM proproteins comprise human heavy chain variable gene segments that are rearranged in vivo within mouse B cells that have been previously engineered to express a limited human light chain repertoire, or a single human light chain, cognate with human heavy chains and, in response to exposure with an antigen of interest, generate an antibody repertoire containing a plurality of human VHs that are cognate with one or one of two possible human VLs, wherein the antibody repertoire specific for the antigen of interest. Common light chains are those derived from a rearranged human Vκ1-39Jκ5 sequence or a rearranged human Vκ3-20Jκ1 sequence, and include somatically mutated (e.g., affinity matured) versions. See, for example, U.S. Pat. No. 10,412,940.

6.9.2. scFv

Single chain Fv or “scFv” antibody fragments comprise the VH and VL domains of an antibody in a single polypeptide chain, are capable of being expressed as a single chain polypeptide, and retain the specificity of the intact antibodies from which they are derived. Generally, the scFv polypeptide further comprises a polypeptide linker between the VH and VL domain that enables the scFv to form the desired structure for target binding. Examples of linkers suitable for connecting the VH and VL chains of an scFv are the non-cleavable linkers identified in Section 6.5.

Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The scFv can comprise VH and VL sequences from any suitable species, such as murine, human or humanized VH and VL sequences.

To create an scFv-encoding nucleic acid, the VH and VL-encoding DNA fragments are operably linked to another fragment encoding a linker, e.g., encoding any of the linkers described in Section 6.5 (typically a repeat of a sequence containing the amino acids glycine and serine, such as the amino acid sequence (Gly4˜Ser) 3 (SEQ ID NO: 156), such that the VH and VL sequences can be expressed as a contiguous single-chain protein, with the VL and VH regions joined by the flexible linker (see, e.g., Bird et al., 1988, Science 242:423-426; Huston et al., 1988, Proc. Natl. Acad. Sci. USA 85:5879-5883; McCafferty et al., 1990, Nature 348:552-554).

### 6.10. Fc Regions

The MBM proproteins of the disclosure typically include a pair of Fc domains that associate to form an Fc region. In native antibodies, Fc regions comprise hinge regions at their N-termini to form a constant domain. Throughout this disclosure, the reference to an Fc domain encompasses an Fc domain with a hinge domain at its N-terminus unless specified otherwise.

The Fc domains can be derived from any suitable species operably linked to an ABS or component thereof. In one embodiment the Fc domain is derived from a human Fc domain. In preferred embodiments, the ABS or component thereof is fused to an IgG Fc molecule. An ABS or component thereof may be fused to the N-terminus or the C-terminus of the IgG Fc domain or both.

The Fc domains can be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domain is derived from IgG1, IgG2, IgG3 or IgG4. In one embodiment the Fc domain is derived from IgG1. In one embodiment the Fc domain is derived from IgG4. Exemplary sequences of Fc domains from IgG1, IgG2, IgG3, and IgG4 are provided in Table Y, below.

TABLE Y
Fc Sequences

| | | SEQ |
|---|---|---|
| Fc | Sequence | ID NO |
| hIgG1 Fc | EPKSCDKTHTCPPCPAPELLGGPSVFLFPPKPKDTLMISRTPEVT | 335 |
| (amino acids | CVVVDVSHEDPEVKFNWYVDGVEVHNAKTKPREEQYNSTYRVV | |
| 99-330 of | SVLTVLHQDWLNGKEYKCKVSNKALPAPIEKTISKAKGQPREPQV | |
| UniprotKB | YTLPPSRDELTKNQVSLTCLVKGFYPSDIAVEWESNGQPENNYKT | |
| P01857-1) | TPPVLDSDGSFFLYSKLTVDKSRWQQGNVFSCSVMHEALHNHYT | |
| | QKSLSLSPGK | |
| hIgG2 Fc | ERKCCVECPPCPAPPVAGPSVFLFPPKPKDTLMISRTPEVTCVVV | 336 |
| (amino acids | DVSHEDPEVQFNWYVDGVEVHNAKTKPREEQFNSTFRVVSVLTV | |
| 99-326 of | VHQDWLNGKEYKCKVSNKGLPAPIEKTISKTKGQPREPQVYTLPP | |
| UniprotKB | SREEMTKNQVSLTCLVKGFYPSDISVEWESNGQPENNYKTTPPM | |
| P01859-1) | LDSDGSFFLYSKLTVDKSRWQQGNVFSCSVMHEALHNHYTQKSL | |
| | SLSPGK | |
| hIgG3 Fc | ELKTPLGDTTHTCPRCPEPKSCDTPPPCPRCPEPKSCDTPPPCP | 337 |
| (amino acids | RCPEPKSCDTPPPCPRCPAPELLGGPSVFLFPPKPKDTLMISRTP | |
| 99-377 of | EVTCWVDVSHEDPEVQFKWYVDGVEVHNAKTKPREEQYNSTF | |
| UniprotKB | RWVSVLTVLHQDWLNGKEYKCKVSNKALPAPIEKTISKTKGQPRE | |
| P01860-1) | PQVYTLPPSREEMTKNQVSLTCLVKGFYPSDIAVEWESSGQPEN | |
| | NYNTTPPMLDSDGSFFLYSKLTVDKSRWQQGNIFSCSVMHEALH | |
| | NRFTQKSLSLSPGK | |
| hIgG4 Fc | ESKYGPPCPSCPAPEFLGGPSVFLFPPKPKDTLMISRTPEVTCVV | 338 |
| (amino acids | VDVSQEDPEVQFNWYVDGVEVHNAKTKPREEQFNSTYRVVSVL | |
| 99-327 of | TVLHQDWLNGKEYKCKVSNKGLPSSIEKTISKAKGQPREPQVYTL | |
| UniprotKB | PPSQEEMTKNQVSLTCLVKGFYPSDIAVEWESNGQPENNYKTTP | |
| P01861-1) | PVLDSDGSFFLYSRLTVDKSRWQEGNVFSCSVMHEALHNHYTQ | |
| | KSLSLSLGK | |

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:336. In cases where an Fc domain comprises at least 90% sequence identity and less than 100% sequence identity to SEQ ID NO: 336 (e.g., between 90% and 99% sequence identity to SEQ ID NO:336), an Fc domain may also comprise one or more amino acid substitutions described herein, for example one or more substitutions that reduce effector function (e.g., as described in Section 6.10.1) and/or one or more substitutions that promote Fc heterodimerization (e.g., as described in Section 6.10.2).

In some aspects, an Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:337. In cases where an Fc domain comprises at least 90% sequence identity and less than 100% sequence identity to SEQ ID NO: 337 (e.g., between 90% and 99% sequence identity to SEQ ID NO:337), an Fc domain may also comprise one or more amino acid substitutions described herein, for example one or more substitutions that reduce effector function (e.g., as described in Section 6.10.1) and/or one or more substitutions that promote Fc heterodimerization (e.g., as described in Section 6.10.2).

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:338. In cases where an Fc domain comprises at least 90% sequence identity and less than 100% sequence identity to SEQ ID NO: 338 (e.g., between 90% and 99% sequence identity to SEQ ID NO:338), an Fc domain may also comprise one or more amino acid substitutions described herein, for example one or more substitutions that reduce effector function (e.g., as described in Section 6.10.1) and/or one or more substitutions that promote Fc heterodimerization (e.g., as described in Section 6.10.2).

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:339. In cases where an Fc domain comprises at least 90% sequence identity and less than 100% sequence identity to SEQ ID NO: 339 (e.g., between 90% and 99% sequence identity to SEQ ID NO:339), an Fc domain may also comprise one or more amino acid substitutions described herein, for example one or more substitutions that reduce effector function (e.g., as described in Section 6.10.1) and/or one or more substitutions that promote Fc heterodimerization (e.g., as described in Section 6.10.2).

The two Fc domains within the Fc region can be the same or different from one another. In a native antibody the Fc domains are typically identical, but for the purpose of producing multispecific binding molecules, e.g., the MBM proproteins of the disclosure and MBMs produced by their activation, the Fc domains might advantageously be different to allow for heterodimerization, as described in Section 6.10.2 below.

In native antibodies, the heavy chain Fc domain of IgA, IgD and IgG is composed of two heavy chain constant domains (CH2 and CH3) and that of IgE and IgM is composed of three heavy chain constant domains (CH2, CH3 and CH4). These dimerize to create an Fc region.

In MBM proproteins of the present disclosure, the Fc region, and/or the Fc domains within it, can comprise heavy chain constant domains from one or more different classes of antibody, for example one, two or three different classes.

In one embodiment the Fc region comprises CH2 and CH3 domains derived from IgG1. One or both of the Fc domains of the Fc region may have a deletion of one or more amino acids relative to a naturally occurring IgG1 CH3 sequence. In some embodiments, an Fc domain of the disclosure has a deletion (e.g., a truncation) of 1, 2, 3, 4, 5, 6, 7, 8, 9, 10, or more amino acids at or near the C-terminus of the CH3 domain relative to a naturally occurring IgG1.

In one embodiment the Fc region comprises CH2 and CH3 domains derived from IgG2. One or both of the Fc domains of the Fc region may have a deletion of one or more amino acids relative to a naturally occurring IgG2 CH3 sequence. In some embodiments, an Fc domain of the disclosure has a deletion (e.g., a truncation) of 1, 2, 3, 4, 5, 6, 7, 8, 9, 10, or more amino acids at or near the C-terminus of the CH3 domain relative to a naturally occurring IgG2.

In one embodiment the Fc region comprises CH2 and CH3 domains derived from IgG3. One or both of the Fc domains of the Fc region may have a deletion of one or more amino acids relative to a naturally occurring IgG3 CH3 sequence. In some embodiments, an Fc domain of the disclosure has a deletion (e.g., a truncation) of 1, 2, 3, 4, 5, 6, 7, 8, 9, 10, or more amino acids at or near the C-terminus of the CH3 domain relative to a naturally occurring IgG3.

In one embodiment the Fc region comprises CH2 and CH3 domains derived from IgG4. One or both of the Fc domains of the Fc region may have a deletion of one or more amino acids relative to a naturally occurring IgG4 CH3 sequence. In some embodiments, an

Fc domain of the disclosure has a deletion (e.g., a truncation) of 1, 2, 3, 4, 5, 6, 7, 8, 9, 10, or more amino acids at or near the C-terminus of the CH3 domain relative to a naturally occurring IgG4.

In one embodiment the Fc region comprises a CH4 domain from IgM. The IgM CH4 domain is typically located at the C-terminus of the CH3 domain.

In one embodiment the Fc region comprises CH2 and CH3 domains derived from IgG and a CH4 domain derived from IgM.

It will be appreciated that the heavy chain constant domains for use in producing an Fc region for the MBM proproteins of the present disclosure may include variants of the naturally occurring constant domains described above. Such variants may comprise one or more amino acid variations compared to wild type constant domains. In one example the Fc region of the present disclosure comprises at least one constant domain that varies in sequence from the wild-type constant domain. It will be appreciated that the variant constant domains may be longer or shorter than the wild-type constant domain. Preferably the variant constant domains are at least 60% identical or similar to a wild-type constant domain. In another example the variant constant domains are at least 70% identical or similar. In another example the variant constant domains are at least 80% identical or similar. In another example the variant constant domains are at least 90% identical or similar. In another example the variant constant domains are at least 95% identical or similar.

Certain variant constant domains contemplated herein include those having a deletion (e.g., truncation) of one or more amino acids at or near (e.g., within 10 amino acids of) the C-terminus relative to a wild-type constant domain. In some embodiments, a constant domain of the present disclosure comprises a deletion of 1, 2, 3, 4, 5, 6, 7, 8, 9, or 10 amino acids at or near the C-terminus relative to a wild-type constant domain.

IgM and IgA occur naturally in humans as covalent multimers of the common H2L2 antibody unit. IgM occurs as a pentamer when it has incorporated a J-chain, or as a hexamer when it lacks a J-chain. IgA occurs as monomer and dimer forms. The heavy chains of IgM and IgA possess an 18 amino acid extension to the C-terminal constant domain, known as a tailpiece. The tailpiece includes a cysteine residue that forms a disulfide bond between heavy chains in the polymer, and is believed to have an important role in polymerization. The tailpiece also contains a glycosylation site. In certain embodiments, the MBM proproteins of the present disclosure do not comprise a tailpiece.

The Fc domains that are incorporated into the MBM proproteins of the present disclosure may comprise one or more modifications that alter the functional properties of the proteins, for example, binding to Fc-receptors such as FcRn or leukocyte receptors, binding to complement, modified disulfide bond architecture, or altered glycosylation patterns. Exemplary Fc modifications that alter effector function are described in Section 6.10.1.

The Fc domains can also be altered to include modifications that improve manufacturability of asymmetric MBM proproteins, for example by allowing heterodimerization, which is the preferential pairing of non-identical Fc domains over identical Fc domains. Heterodimerization permits the production of MBM proproteins in which different polypeptide components are connected to one another by an Fc region containing Fc domains that differ in sequence. Examples of heterodimerization strategies are exemplified in Section 6.10.2.

It will be appreciated that any of the modifications mentioned above can be combined in any suitable manner to achieve the desired functional properties and/or combined with other modifications to alter the properties of the MBM proproteins.

6.10.1. Fc Domains with Altered Effector Function

In some embodiments, the Fc domain comprises one or more amino acid substitutions that reduces binding to an Fc receptor and/or effector function.

In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

In one embodiment, the Fc domain or the Fc region (e.g., one or both Fc domains of an MBM proprotein that can associate to form an Fc region) comprises an amino acid substitution at a position selected from the group of E233, L234, L235, N297, P331 and P329 (numberings according to Kabat EU index). In a more specific embodiment, the Fc domain or the Fc region comprises an amino acid substitution at a position selected from the group of L234, L235 and P329 (numberings according to Kabat EU index). In some embodiments, the Fc domain or the Fc region comprises the amino acid substitutions L234A and L235A (numberings according to Kabat EU index). In one such embodiment, the Fc domain or region is an Igd Fc domain or region, particularly a human Igd Fc domain or region. In one embodiment, the Fc domain or the Fc region comprises an amino acid substitution at position P329. In a more specific embodiment, the amino acid substitution is P329A or P329G, particularly P329G (numberings according to Kabat EU index). In one embodiment, the Fc domain or the Fc region comprises an amino acid substitution at position P329 and a further amino acid substitution at a position selected from E233, L234, L235, N297 and P331 (numberings according to Kabat EU index). In a more specific embodiment, the further amino acid substitution is E233P, L234A, L235A, L235E, N297A, N297D or P331S. In particular embodiments, the Fc domain or the Fc region comprises amino acid substitutions at positions P329, L234 and L235 (numberings according to Kabat EU index). In more particular embodiments, the Fc domain comprises the amino acid mutations L234A, L235A and P329G (“P329G LALA”, “PGLALA” or “LALAPG”).

Typically, the same one or more amino acid substitution is present in each of the two Fc domains of an Fc region. Thus, in a particular embodiment, each Fc domain of the Fc region comprises the amino acid substitutions L234A, L235A and P329G (Kabat EU index numbering), i.e. in each of the first and the second Fc domains in the Fc region the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In one embodiment, the Fc domain is an IgG1 Fc domain, particularly a human IgG1 Fc domain. In some embodiments, the IgG1 Fc domain is a variant IgG1 comprising D265A, N297A mutations (EU numbering) to reduce effector function.

In another embodiment, the Fc domain is an IgG4 Fc domain with reduced binding to Fc receptors. Exemplary IgG4 Fc domains with reduced binding to Fc receptors may comprise an amino acid sequence selected from Table I below: In some embodiments, the Fc domain includes only the bolded portion of the sequences shown below:

TABLE I

| Fc Domain | Sequence |
|---|---|
| SEQ ID NO: 323 | Asp Lys Arg Val Glu Ser Lys Tyr Gly Pro Cys Pro Pro Cys |
| (SEQ ID NO: 1 of | Pro Ala Pro Pro Val Ala Gly Pro Ser Val Phe Leu Phe Pro |
| WO2014/121087) | Pro Lys Pro Lys Asp Thr Leu Met lle Ser Arg Thr Pro Glu |
| | Val Thr Cys Val Val Val Asp Val Ser Gln Glu Asp Pro Glu |
| | Val Gln Phe Asn Trp Tyr Val Asp Gly Val Glu Val His Asn |
| | Ala Lys Thr Lys Pro Arg Glu Glu Gln Phe Asn Ser Thr Tyr |
| | Arg Val Val Ser Val Leu Thr Val Leu His Gln Asp Trp Leu |
| | Asn Gly Lys Glu Tyr Lys Cys Lys Val Ser Asn Lys Gly Leu |
| | Pro Ser Ser lle Glu Lys Thr lle Ser Lys Ala Lys Gly Gln Pro |
| | Arg Glu Pro Gln Val Tyr Thr Leu Pro Pro Ser Gln Glu Glu |
| | Met Thr Lys Asn Gln Val Ser Leu Thr Cys Leu Val Lys Gly |
| | Phe Tyr Pro Ser Asp lle Ala Val Glu Trp Glu Ser Asn Gly |
| | Gln Pro Glu Asn Asn Tyr Lys Thr Thr Pro Pro Val Leu Asp |
| | Ser Asp Gly Ser Phe Phe Leu Tyr Ser Arg Leu Thr Val Asp |
| | Lys Ser Arg Trp Gln Glu Gly Asn Val Phe Ser Cys Ser Val |
| | Met His Glu Ala Leu His Asn His Tyr Thr Gln Lys Ser Leu |
| | Ser Leu Ser Leu Gly Lys |
| SEQ ID NO: 324 | Asp Lys Lys Val Glu Pro Lys Ser Cys Asp Lys Thr His Thr |
| (SEQ ID NO: 2 of | Cys Pro Pro Cys Pro Ala Pro Pro Val Ala Gly Pro Ser Val |
| WO2014/121087) | Phe Leu Phe Pro Pro Lys Pro Lys Asp Thr Leu Met lle Ser |
| | Arg Thr Pro Glu Val Thr Cys Val Val Val Asp Val Ser Gln |
| | Glu Asp Pro Glu Val Gln Phe Asn Trp Tyr Val Asp Gly Val |
| | Glu Val His Asn Ala Lys Thr Lys Pro Arg Glu Glu Gln Phe |
| | Asn Ser Thr Tyr Arg Val Val Ser Val Leu Thr Val Leu His |
| | Gln Asp Trp Leu Asn Gly Lys Glu Tyr Lys Cys Lys Val Ser |
| | Asn Lys Gly Leu Pro Ser Ser lle Glu Lys Thr lle Ser Lys Ala |
| | Lys Gly Gln Pro Arg Glu Pro Gln Val Tyr Thr Leu Pro Pro |
| | Ser Arg Asp Glu Leu Thr Lys Asn Gln Val Ser Leu Thr Cys |
| | Leu Val Lys Gly Phe Tyr Pro Ser Asp lle Ala Val Glu Trp Glu |
| | Ser Asn Gly Gln Pro Glu Asn Asn Tyr Lys Thr Thr Pro Pro |
| | Val Leu Asp Ser Asp Gly Ser Phe Phe Leu Tyr Ser Lys Leu |
| | Thr Val Asp Lys Ser Arg Trp Gln Gln Gly Asn Val Phe Ser |
| | Cys Ser Val Met His Glu Ala Leu His Asn His Tyr Thr Gln |
| | Lys Ser Leu Ser Leu Ser Pro Gly Lys |
| SEQ ID NO: 325 | Ala Ser Thr Lys Gly Pro Ser Val Phe Pro Leu Ala Pro Ser |
| (SEQ ID NO: 30 | Ser Lys |
| of | Ser Thr Ser Gly Gly Thr Ala Ala Leu Gly Cys Leu Val Lys |
| WO2014/121087) | Asp Tyr |
| | Phe Pro Glu Pro Val Thr Val Ser Trp Asn Ser Gly Ala Leu |
| | Thr Ser Gly Val His Thr Phe Pro Ala Val Leu Gln Ser Ser |
| | Gly Leu Tyr Ser Leu Ser Ser Val Val Thr Val Pro Ser Ser |
| | Ser Leu Gly Thr Gln Thr Tyr lle Cys Asn Val Asn His Lys |
| | Pro Ser Asn Thr Lys Val Asp Lys Lys Val Glu Pro Lys Ser |
| | Cys Asp Lys Thr His Thr Cys Pro Pro Cys Pro Ala Pro |
| | Pro Val Ala Gly Pro Ser Val Phe Leu Phe Pro Pro Lys |
| | Pro Lys Asp Thr Leu Met lle Ser Arg Thr Pro Glu Val Thr |
| | Cys Val Val Val Asp Val Ser Gln Glu Asp Pro Glu Val Gln |
| | Phe Asn Trp Tyr Val Asp Gly Val Glu Val His Asn Ala |
| | Lys Thr Lys Pro Arg Glu Glu Gln Phe Asn Ser Thr Tyr |
| | Arg Val Val Ser Val Leu Thr Val Leu His Gln Asp Trp Leu |
| | Asn Gly Lys Glu Tyr Lys Cys Lys Val Ser Asn Lys Gly |
| | Leu Pro Ser Ser lle Glu Lys Thr lle Ser Lys Ala Lys Gly |
| | Gln Pro Arg Glu Pro Gln Val Tyr Thr Leu Pro Pro Ser Arg |
| | Asp Glu Leu Thr Lys Asn Gln Val Ser Leu Thr Cys Leu |
| | Val Lys Gly Phe Tyr Pro Ser Asp lle Ala Val Glu Trp Glu |
| | Ser Asn Gly Gln Pro Glu Asn Asn |
| | Tyr Lys Thr Thr Pro Pro Val Leu Asp Ser Asp Gly Ser |
| | Phe Phe Leu Tyr Ser Lys Leu Thr Val Asp Lys Ser Arg |
| | Trp Gln Gln Gly Asn Val Phe Ser Cys Ser Val Met His |
| | Glu Ala Leu His Asn His Tyr Thr Gln Lys Ser Leu Ser |
| | Leu Ser Pro Gly Lys |
| SEQ ID NO: 326 | Ala Ser Thr Lys Gly Pro Ser Val Phe Pro Leu Ala Pro Cys |
| (SEQ ID NO: 31 | Ser Arg Ser Thr Ser Glu Ser Thr Ala Ala Leu Gly Cys Leu |
| of | Val Lys Asp Tyr Phe Pro Glu Pro Val Thr Val Ser Trp Asn |
| WO2014/121087) | Ser Gly Ala Leu Thr Ser Gly Val His Thr Phe Pro Ala Val |
| | Leu Gln Ser Ser Gly Leu Tyr Ser Leu Ser Ser Val Val Thr |
| | Val Pro Ser Ser Ser Leu Gly Thr Lys Thr Tyr Thr Cys Asn |
| | Val Asp His Lys Pro Ser Asn Thr Lys Val Asp Lys Arg Val |
| | Glu Ser Lys Tyr Gly Pro Pro Cys Pro Pro Cys Pro Ala |
| | Pro Pro Val Ala Gly Pro Ser Val Phe Leu Phe Pro Pro |
| | Lys Pro Lys Asp Thr Leu Met lle Ser Arg Thr Pro Glu Val |
| | Thr Cys Val Val Val Asp Val Ser Gln Glu Asp Pro Glu Val |
| | Gln Phe Asn Trp Tyr Val Asp Gly Val Glu Val His Asn |
| | Ala Lys Thr Lys Pro Arg Glu Glu Gln Phe Asn Ser Thr |
| | Tyr Arg Val Val Ser Val Leu Thr Val Leu His Gln Asp Trp |
| | Leu Asn Gly Lys Glu Tyr Lys Cys Lys Val Ser Asn Lys |
| | Gly Leu Pro Ser Ser lle Glu Lys Thr lle Ser Lys Ala Lys |
| | Gly Gln Pro Arg Glu Pro Gln Val Tyr Thr Leu Pro Pro Ser |
| | Gln Glu Glu Met Thr Lys Asn |
| | Gln Val Ser Leu Thr Cys Leu Val Lys Gly Phe Tyr Pro |
| | Ser Asp lle Ala Val Glu Trp Glu Ser Asn Gly Gln Pro Glu |
| | Asn Asn Tyr Lys Thr Thr Pro Pro Val Leu Asp Ser Asp |
| | Gly Ser Phe Phe Leu Tyr Ser Arg Leu Thr Val Asp Lys |
| | Ser Arg Trp Gln Glu Gly Asn Val Phe Ser Cys Ser Val |
| | Met His Glu Ala Leu His Asn His Tyr Thr Gln Lys Ser |
| | Leu Ser Leu Ser Leu Gly Lys |
| SEQ ID NO: 327 | Ala Ser Thr Lys Gly Pro Ser Val Phe Pro Leu Ala Pro Ser |
| (SEQ ID NO: 37 | Ser Lys |
| of | Ser Thr Ser Gly Gly Thr Ala Ala Leu Gly Cys Leu Val Lys |
| WO2014/121087) | Asp Tyr |
| | Phe Pro Glu Pro Val Thr Val Ser Trp Asn Ser Gly Ala Leu |
| | Thr Ser Gly Val His Thr Phe Pro Ala Val Leu Gln Ser Ser |
| | Gly Leu Tyr Ser Leu Ser Ser Val Val Thr Val Pro Ser Ser |
| | Ser Leu Gly Thr Gln Thr Tyr lle Cys Asn Val Asn His Lys |
| | Pro Ser Asn Thr Lys Val Asp Lys Lys Val Glu Pro Lys Ser |
| | Cys Asp Lys Thr His Thr Cys Pro Pro Cys Pro Ala Pro |
| | Pro Val Ala Gly Pro Ser Val Phe Leu Phe Pro Pro Lys |
| | Pro Lys Asp Thr Leu Met lle Ser Arg Thr Pro Glu Val Thr |
| | Cys Val Val Val Asp Val Ser Gln Glu Asp Pro Glu Val Gln |
| | Phe Asn Trp Tyr Val Asp Gly Val Glu Val His Asn Ala |
| | Lys Thr Lys Pro Arg Glu Glu Gln Phe Asn Ser Thr Tyr |
| | Arg Val Val Ser Val Leu Thr Val Leu His Gln Asp Trp Leu |
| | Asn Gly Lys Glu Tyr Lys Cys Lys Val Ser Asn Lys Gly |
| | Leu Pro Ser Ser lle Glu Lys Thr lle Ser Lys Ala Lys Gly |
| | Gln Pro Arg Glu Pro Gln Val Tyr Thr Leu Pro Pro Ser Arg |
| | Asp Glu Leu Thr Lys Asn Gln Val Ser Leu Thr Cys Leu |
| | Val Lys Gly Phe Tyr Pro Ser Asp lle Ala Val Glu Trp Glu |
| | Ser Asn Gly Gln Pro Glu Asn Asn |
| | Tyr Lys Thr Thr Pro Pro Val Leu Asp Ser Asp Gly Ser |
| | Phe Phe Leu Tyr Ser Lys Leu Thr Val Asp Lys Ser Arg |
| | Trp Gln Gln Gly Asn Val Phe Ser Cys Ser Val Met His |
| | Glu Ala Leu His Asn Arg Phe Thr Gln Lys Ser Leu Ser |
| | Leu Ser Pro Gly Lys |
| SEQ ID NO: 328 | Ala Ser Thr Lys Gly Pro Ser Val Phe Pro Leu Ala Pro Cys |
| (SEQ ID NO: 38 | Ser Arg Ser Thr Ser Glu Ser Thr Ala Ala Leu Gly Cys Leu |
| of | Val Lys Asp Tyr Phe Pro Glu Pro Val Thr Val Ser Trp Asn |
| WO2014/121087) | Ser Gly Ala Leu Thr Ser Gly Val His Thr Phe Pro Ala Val |
| | Leu Gln Ser Ser Gly Leu Tyr Ser Leu Ser Ser Val Val Thr |
| | Val Pro Ser Ser Ser Leu Gly Thr Lys Thr Tyr Thr Cys Asn |
| | Val Asp His Lys Pro Ser Asn Thr Lys Val Asp Lys Arg Val |
| | Glu Ser Lys Tyr Gly Pro Pro Cys Pro Pro Cys Pro Ala |
| | Pro Pro Val Ala Gly Pro Ser Val Phe Leu Phe Pro Pro |
| | Lys Pro Lys Asp Thr Leu Met lle Ser Arg Thr Pro Glu Val |
| | Thr Cys Val Val Val Asp Val Ser Gln Glu Asp Pro Glu Val |
| | Gln Phe Asn Trp Tyr Val Asp Gly Val Glu Val His Asn |
| | Ala Lys Thr Lys Pro Arg Glu Glu Gln Phe Asn Ser Thr |
| | Tyr Arg Val Val Ser Val Leu Thr Val Leu His Gln Asp Trp |
| | Leu Asn Gly Lys Glu Tyr Lys Cys Lys Val Ser Asn Lys |
| | Gly Leu Pro Ser Ser lle Glu Lys Thr lle Ser Lys Ala Lys |
| | Gly Gln Pro Arg Glu Pro Gln Val Tyr Thr Leu Pro Pro Ser |
| | Gln Glu Glu Met Thr Lys Asn |
| | Gln Val Ser Leu Thr Cys Leu Val Lys Gly Phe Tyr Pro |
| | Ser Asp lle Ala Val Glu Trp Glu Ser Asn Gly Gln Pro Glu |
| | Asn Asn Tyr Lys Thr Thr Pro Pro Val Leu Asp Ser Asp |
| | Gly Ser Phe Phe Leu Tyr Ser Arg Leu Thr Val Asp Lys |
| | Ser Arg Trp Gln Glu Gly Asn Val Phe Ser Cys Ser Val |
| | Met His Glu Ala Leu His Asn Arg Phe Thr Gln Lys Ser |
| | Leu Ser Leu Ser Leu Gly Lys |

In a particular embodiment, the IgG4 with reduced effector function comprises the bolded portion of the amino acid sequence of SEQ ID NO:31 of WO2014/121087, sometimes referred to herein as IgG4s or hIgG4s.

For heterodimeric Fc regions, it is possible to incorporate a combination of the variant IgG4 Fc sequences set forth above, for example an Fc region comprising an Fc domain comprising the amino acid sequence of SEQ ID NO:30 of WO2014/121087 (or the bolded portion thereof) and an Fc domain comprising the amino acid sequence of SEQ ID NO:37 of WO2014/121087 (or the bolded portion thereof) or an Fc region comprising an Fc domain comprising the amino acid sequence of SEQ ID NO:31 of WO2014/121087 (or the bolded portion thereof) and an Fc domain comprising the amino acid sequence of SEQ ID NO:38 of WO2014/121087 (or the bolded portion thereof).

### 6.10.2. Fc Heterodimerization Variants

Certain MBM proproteins entail dimerization between two Fc domains that, unlike a native immunoglobulin, are operably linked to non-identical N-terminal regions, e.g., one Fc domain connected to a Fab and the other Fc domain connected to an ABS or component thereof. Inadequate heterodimerization of two Fc domains to form an Fc region has can be an obstacle for increasing the yield of desired heterodimeric molecules and represents challenges for purification. A variety of approaches available in the art can be used in for enhancing dimerization of Fc domains that might be present in the MBM proproteins of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO 2009/089004A1.

The present disclosure provides MBM proproteins comprising Fc heterodimers, i.e., Fc regions comprising heterologous, non-identical Fc domains. Typically, each Fc domain in the Fc heterodimer comprises a CH3 domain of an antibody. The CH3 domains are derived from the constant region of an antibody of any isotype, class or subclass, and preferably of IgG (IgG1, IgG2, IgG3 and IgG4) class, as described in the preceding section.

Heterodimerization of the two different heavy chains at CH3 domains give rise to the desired MBM proprotein, while homodimerization of identical heavy chains will reduce yield of the desired MBM proprotein. Thus, in a preferred embodiment, the polypeptides that associate to form an MBM proprotein of the disclosure will contain CH3 domains with modifications that favor heterodimeric association relative to unmodified Fc domains.

In a specific embodiment said modification promoting the formation of Fc heterodimers is a so-called “knob-into-hole” or “knob-in-hole” modification, comprising a “knob” modification in one of the Fc domains and a “hole” modification in the other Fc domain. The knob-into-hole technology is described e.g., in U.S. Pat. Nos. 5,731,168; 7,695,936; Ridgway et al., 1996, Prot Eng 9:617-621, and Carter, 2001, Immunol Meth 248:7-15. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in some embodiments, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine(S), threonine (T), and valine (V). The protuberance and cavity can be made by altering the nucleic acid encoding the polypeptides, e.g., by site-specific mutagenesis, or by peptide synthesis. An exemplary substitution is Y470T.

In a specific such embodiment, in the first Fc domain the threonine residue at position 366 is replaced with a tryptophan residue (T366W), and in the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further embodiment, in the first Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second Fc domain additionally the tyrosine residue at position 349 is replaced by a cysteine residue (Y349C) (numbering according to Kabat EU index). In a particular embodiment, the first Fc domain comprises the amino acid substitutions S354C and T366W, and the second Fc domain comprises the amino acid substitutions Y349C, T366S, L368A and Y407V (numbering according to Kabat EU index).

In some embodiments, electrostatic steering (e.g., as described in Gunasekaran et al., 2010, J Biol Chem 285 (25): 19637-46) can be used to promote the association of the first and the second Fc domains of the Fc region.

As an alternative, or in addition, to the use of Fc domains that are modified to promote heterodimerization, an Fc domain can be modified to allow a purification strategy that enables selections of Fc heterodimers. In one such embodiment, one polypeptide comprises a modified Fc domain that abrogates its binding to Protein A, thus enabling a purification method that yields a heterodimeric protein. See, for example, U.S. Pat. No. 8,586,713. As such, the MBM proproteins comprise a first CH3 domain and a second Ig CH3 domain, wherein the first and second Ig CH3 domains differ from one another by at least one amino acid, and wherein at least one amino acid difference reduces binding of the MBM proprotein to Protein A as compared to a corresponding MBM proprotein lacking the amino acid difference. In one embodiment, the first CH3 domain binds Protein A and the second CH3 domain contains a mutation/modification that reduces or abolishes Protein A binding such as an H95R modification (by IMGT exon numbering; H435R by EU numbering). The second CH3 may further comprise a Y96F modification (by IMGT; Y436F by EU). This class of modifications is referred to herein as “star” mutations.

In some embodiments, the Fc can contain one or more mutations (e.g., knob and hole mutations) to facilitate heterodimerization as well as star mutations to facilitate purification.

### 6.10.3. Hinge Domains

The MBM proproteins of the disclosure can comprise an Fc domain comprising a hinge domain at its N-terminus. The hinge region can be a native or a modified hinge region. Hinge regions are typically found at the N-termini of Fc regions. The term “hinge domain”, unless the context dictates otherwise, refers to a naturally or non-naturally occurring hinge sequence that in the context of a single or monomeric polypeptide chain is a monomeric hinge domain and in the context of a dimeric polypeptide (e.g., a homodimeric or heterodimeric MBM proprotein formed by the association of two Fc domains) can comprise two associated hinge sequences on separate polypeptide chains. Sometimes, the two associated hinge sequences are referred to as a “hinge reion”.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama or goat hinge regions. Other modified hinge regions may comprise a complete hinge region derived from an antibody of a different class or subclass from that of the heavy chain Fc domain or Fc region. Alternatively, the modified hinge region may comprise part of a natural hinge or a repeating unit in which each unit in the repeat is derived from a natural hinge region. In a further alternative, the natural hinge region may be altered by converting one or more cysteine or other residues into neutral residues, such as serine or alanine, or by converting suitably placed residues into cysteine residues. By such means the number of cysteine residues in the hinge region may be increased or decreased. Other modified hinge regions may be entirely synthetic and may be designed to possess desired properties such as length, cysteine composition and flexibility.

A number of modified hinge regions have already been described for example, in U.S. Pat. No. 5,677,425, WO 99/15549, WO 2005/003170, WO 2005/003169, WO 2005/003170, WO 98/25971 and WO 2005/003171 and these are incorporated herein by reference.

In one embodiment, an MBM proprotein of the disclosure comprises an Fc region in which one or both Fc domains possesses an intact hinge domain at its N-terminus.

In various embodiments, positions 233-236 within a hinge region may be G, G, G and unoccupied; G, G, unoccupied, and unoccupied; G, unoccupied, unoccupied, and unoccupied; or all unoccupied, with positions numbered by EU numbering.

In some embodiments, the MBM proproteins of the disclosure comprise a modified hinge region that reduces binding affinity for an Fcγ receptor relative to a wild-type hinge region of the same isotype (e.g., human IgG1 or human IgG4).

In one embodiment, the MBM proproteins of the disclosure comprise an Fc region in which each Fc domain possesses an intact hinge domain at its N-terminus, where each Fc domain and hinge domain is derived from IgG4 and each hinge domain comprises the modified sequence CPPC (SEQ ID NO: 329). The core hinge region of human IgG4 contains the sequence CPSC (SEQ ID NO: 330) compared to IgG1 that contains the sequence CPPC (SEQ ID NO: 334). The serine residue present in the IgG4 sequence leads to increased flexibility in this region, and therefore a proportion of molecules form disulfide bonds within the same protein chain (an intrachain disulfide) rather than bridging to the other heavy chain in the IgG molecule to form the interchain disulfide. (Angel et al., 1993, Mol Immunol 30 (1): 105-108). Changing the serine residue to a proline to give the same core sequence as IgG1 allows complete formation of inter-chain disulfides in the IgG4 hinge region, thus reducing heterogeneity in the purified product. This altered isotype is termed IgG4P.

### 6.10.3.1. Chimeric Hinge Sequences

The hinge domain can be a chimeric hinge domain.

For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region.

In particular embodiments, a chimeric hinge region comprises the amino acid sequence EPKSCDKTHTCPPCPAPPVA (SEQ ID NO: 331) (previously disclosed as SEQ ID NO: 8 of WO2014/121087, which is incorporated by reference in its entirety herein) or ESKYGPPCPPCPAPPVA (SEQ ID NO: 332) (previously disclosed as SEQ ID NO:9 of WO2014/121087). Such chimeric hinge sequences can be suitably linked to an IgG4 CH2 region (for example by incorporation into an IgG4 Fc domain, for example a human or murine Fc domain, which can be further modified in the CH2 and/or CH3 domain to reduce effector function, for example as described in Section 6.10.1).

6.10.3.2. Hinge Sequences with Reduced Effector Function

In further embodiments, the hinge region can be modified to reduce effector function, for example as described in WO2016161010A2, which is incorporated by reference in its entirety herein. In various embodiments, the positions 233-236 of the modified hinge region are G, G, G and unoccupied; G, G, unoccupied, and unoccupied; G, unoccupied, unoccupied, and unoccupied; or all unoccupied, with positions numbered by EU numbering (as shown in FIG. 1 of WO2016161010A2). These segments can be represented as GGG-, GG--, G--- or ---- with “-” representing an unoccupied position.

Position 236 is unoccupied in canonical human IgG2 but is occupied by in other canonical human IgG isotypes. Positions 233-235 are occupied by residues other than G in all four human isotypes (as shown in FIG. 1 of WO2016161010A2).

The hinge modification within positions 233-236 can be combined with position 228 being occupied by P. Position 228 is naturally occupied by P in human IgG1 and IgG2 but is occupied by S in human IgG4 and R in human IgG3. An S228P mutation in an IgG4 antibody is advantageous in stabilizing an IgG4 antibody and reducing exchange of heavy chain light chain pairs between exogenous and endogenous antibodies. Preferably positions 226-229 are occupied by C, P, P and C respectively (“CPPC” disclosed as SEQ ID NO: 329).

Exemplary hinge regions have residues 226-236, sometimes referred to as middle (or core) and lower hinge, occupied by the modified hinge sequences designated GGG-(233-236), GG--(233-236), G---(233-236) and no G(233-236). Optionally, the hinge domain amino acid sequence comprises CPPCPAPGGG-GPSVF (SEQ ID NO: 333) (previously disclosed as SEQ ID NO:1 of WO2016161010A2), CPPCPAPGG--GPSVF (SEQ ID NO: 26) (previously disclosed as SEQ ID NO:2 of WO2016161010A2), CPPCPAPG---GPSVF (SEQ ID NO: 106) (previously disclosed as SEQ ID NO:3 of WO2016161010A2), or CPPCPAP----GPSVF (SEQ ID NO: 152) (previously disclosed as SEQ ID NO:4 of WO2016161010A2).

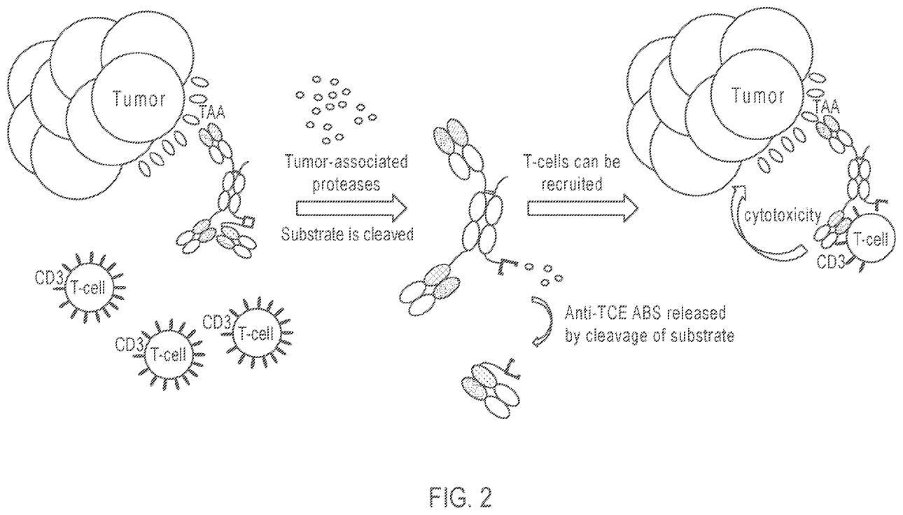

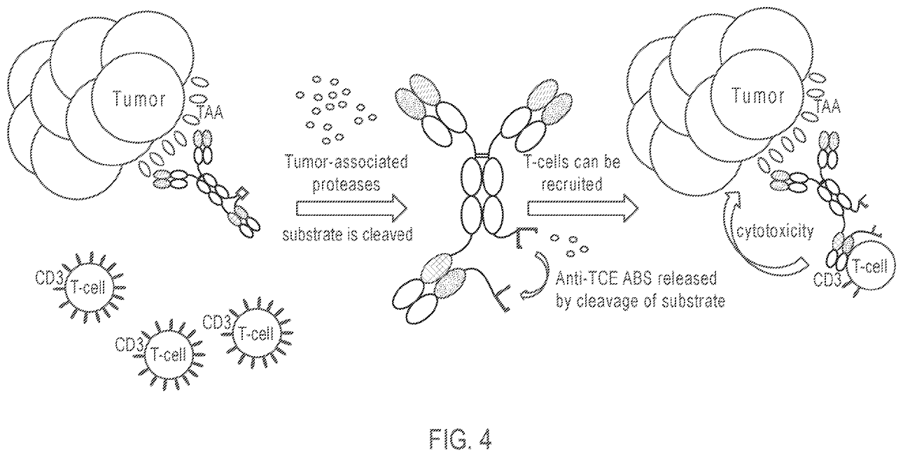

The modified hinge regions described above can be incorporated into a heavy chain constant region, which typically include CH2 and CH3 domains, and which may have an additional hinge segment (e.g., an upper hinge) flanking the designated region. Such additional constant region segments present are typically of the same isotype, preferably a human isotype, although can be hybrids of different isotypes. The isotype of such additional human constant regions segments is preferably human IgG4 but can also be human IgG1, IgG2, or IgG3 or hybrids thereof in which domains are of different isotypes. Exemplary sequences of human IgG1, IgG2 and IgG4 are shown in FIGS. 2-4 of WO2016161010A2. In specific embodiments, the modified hinge sequences can be linked to an IgG4

CH2 region (for example by incorporation into an IgG4 Fc domain, for example a human or murine Fc domain, which can be further modified in the CH2 and/or CH3 domain to reduce effector function, for example as described in Section 6.10.1).

### 6.11. Tandem Fabs

Certain aspects of the disclosure relate to tandem Fab MBMs. The tandem Fabs MBMs comprise a TCE ABS, e.g., a TCE ABS as described in Section 6.7, and a TAA ABS, e.g., a TAA ABS as described in Section 6.6, both in the form of a Fab, e.g., a Fab domain as described in Section 6.9.1. The TCE ABS and TAA ABS can be separated by a non-cleavable linker, for example as described in Section 6.5.

In some aspects, the disclosure provides a tandem Fab MBM comprising (1) a first polypeptide chain comprising from N- to C-terminal orientation a VH of a TCE ABS operably linked to a CH1 domain; an optional linker, e.g., non-cleavable linker; and a VH of a TAA ABS operably linked to a CH1 domain; (2) a second polypeptide chain comprising a VL of the TAA ABS operably linked to a CL domain; and (3) a third polypeptide chain comprising a VL of the TCE ABS operably linked to a CL domain. In some embodiments, the tandem Fab MBM comprises (1) a first polypeptide chain comprising from N- to C-terminal orientation (a) a VH of a TCE ABS; (b) a CH1 domain; (c) a non-cleavable linker; (d) a VH of a TAA ABS; and (c) a CH1 domain; (2) a second polypeptide chain comprising (a) a VL of the TAA ABS and (b) a CL domain; and (3) a third polypeptide chain comprising (a) a VL of the TCE ABS and (b) a CL domain.

In other aspects, the disclosure provides a tandem Fab MBM comprising (1) a first polypeptide chain comprising from N- to C-terminal orientation a VH of a TAA ABS operably linked to a CH1 domain; an optional linker, e.g., non-cleavable linker; and a VH of a TCE ABS operably linked to a CH1 domain; (2) a second polypeptide chain comprising a VL of the TAA ABS operably linked to a CL domain; and (3) a third polypeptide chain comprising a VL of the TCE ABS operably linked to a CL domain. In some embodiments, the tandem Fab MBM comprises (1) a first polypeptide chain comprising from N- to C-terminal orientation (a) a VH of a TAA ABS; (b) a CH1 domain; (c) a non-cleavable linker; (d) a VH of a TCE ABS; and (c) a CH1 domain; (2) a second polypeptide chain comprising (a) a VL of the TAA ABS and (b) a CL domain; and (3) a third polypeptide chain comprising (a) a VL of the TCE ABS and (b) a CL domain.

In some embodiments, the CH1 of the TCE ABS and the CH1 of the TAA ABS are the same. In further embodiments the CL of the TCE ABS and the CL of the TAA ABS are the same. In yet further embodiments, the VL of the TAA ABS and the TCE ABS are the same and are universal light chain VLs. Exemplary universal light chains that can be incorporated into the TAA ABS and TCE ABS are set forth in Table J below.

TABLE J
Exemplary Universal Light Chains

| Reference | Universal Light Chain Name and/or SEQ ID NO(s) |
|---|---|
| WO 2021/244522 A1 | SEQ ID NOs: 38, 39, and 40 |
| WO 2021/173844 A1 | VL of anti-CD38003 |
| WO 2021/123090 A1 | SEQ ID NOs: 101 and 103 |
| US 2021/0206865 A1 | SEQ ID NO: 129 |
| US 2021/0206870 A1 | SEQ ID NO: 3 |
| WO 2020/229621 A1 | 0325L and 0128L |
| US 2020/0199250 A1 | SEQ ID NO: 405 and SEQ ID NO: 414 |
| US 2022/0090125 A1 | SEQ ID NO: 1 and SEQ ID NO: 2 |
| U.S. Pat. No. 11,254,736 B2 | SEQ ID NOS: 1 through 9 |
| US 2021/0179731 A1 | SEQ ID NOS: 17, 36, 200 and 204 |
| WO 2019/190327 A2 | SEQ ID NOS: 29, 35, 37 and 40 |
| U.S. Pat. No. 10,986,820 B2 | ULCs encoded by nucleotides 2362 through 2686 of SEQ |
| | ID NO: 1 and nucleotides 2373 through 2697 of SEQ ID |
| | NO: 11. |
| US 2019/0100771 A1 | SEQ ID NO: 01 |
| WO 2016/079081 A1 | SEQ ID NO: 31 and SEQ ID NO: 35 |
| US 2021/0206865 A1 | VL of SEQ ID NO: 82 |
| | CDR-L1 of SEQ ID NO: 84 |
| | CDR-L2 of SEQ ID NO: 86 |
| | CDR-L3 of SEQ ID NO: 88 |

The optional linkers are preferably selected from the linkers disclosed in Section [0112]. In some embodiments, the linkers are less than 50 amino acids in length amino acids in length (e.g., are 5 to 45 amino acids in length, 10 to 35 amino acids in length, or 5 to 25 amino acids in length) and comprise a glycine-serine sequence, e.g., G4S (SEQ ID NO: 1) or a multimer thereof.

### 6.12. Nucleic Acids and Host Cells

In another aspect, the disclosure provides nucleic acids encoding the MBM proproteins and tandem Fab MBMs of the disclosure. In some embodiments, the MBM proproteins and tandem Fab MBMs are encoded by a single nucleic acid. In other embodiments, the MBM proproteins and tandem Fab MBMs can be encoded by a plurality (e.g., two, three, four or more) nucleic acids.

A single nucleic acid can encode an MBM proprotein or tandem Fab MBM that comprises a single polypeptide chain, an MBM proprotein or tandem Fab MBM that comprises two or more polypeptide chains, or a portion of an MBM proprotein or tandem Fab MBM that comprises more than two polypeptide chains (for example, a single nucleic acid can encode two polypeptide chains of an MBM proprotein or tandem Fab MBM comprising three, four or more polypeptide chains, or three polypeptide chains of an MBM proprotein comprising four or more polypeptide chains). For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements, and separated by internal ribosome entry site (IRES) sequences allowing for translation into separate polypeptides.

In some embodiments, an MBM proprotein or tandem Fab MBM comprising two or more polypeptide chains is encoded by two or more nucleic acids. The number of nucleic acids encoding an MBM proprotein or tandem Fab MBM can be equal to or less than the number of polypeptide chains in the MBM proprotein or tandem Fab MBM (for example, when more than one polypeptide chains are encoded by a single nucleic acid).

The nucleic acids of the disclosure can be DNA or RNA (e.g., mRNA).

In another aspect, the disclosure provides host cells and vectors containing the nucleic acids of the disclosure. The nucleic acids may be present in a single vector or separate vectors present in the same host cell or separate host cell, as described in more detail herein below.

### 6.12.1. Vectors

The disclosure provides vectors comprising nucleotide sequences encoding an MBM proprotein, a tandem Fab MBM or a component thereof described herein, for example one or two of the polypeptide chains of a half antibody of an MBM proprotein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by co-transformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid-based transfection or other conventional techniques. Methods and conditions for culturing the resulting transfected cells and for recovering the expressed polypeptides are known to those skilled in the art, and may be varied or optimized depending upon the specific expression vector and mammalian host cell employed, based upon the present description.

### 6.12.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### 6.13. Pharmaceutical Compositions

The MBM proproteins and tandem Fab MBMs of the disclosure may be in the form of compositions comprising the MBM proprotein or tandem Fab MBM and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the MBM proprotein and tandem Fab MBM and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intratumorally, intrathecally, topically or locally. The most suitable route for administration in any given case will depend on the particular MBM proprotein or tandem Fab MBM, the subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of an MBM proprotein or tandem Fab MBM of the disclosure per dose. The quantity of MBM proprotein or tandem Fab MBM included in a unit dose will depend on the disease being treated, as well as other factors as are well known in the art. Such unit dosages may be in the form of a lyophilized dry powder containing an amount of MBM proprotein or tandem Fab MBM suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of MBM proprotein suitable for a single administration.

The pharmaceutical compositions may also be supplied in bulk from containing quantities of MBM proprotein or tandem Fab MBM suitable for multiple administrations.

Pharmaceutical compositions may be prepared for storage as lyophilized formulations or aqueous solutions by mixing an MBM proprotein or tandem Fab MBM having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives should be nontoxic to the recipients at the dosages and concentrations employed.

Buffering agents help to maintain the pH in the range which approximates physiological conditions. They may be present at a wide variety of concentrations, but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth, and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins: hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 wt % per wt of MBM proprotein or tandem Fab MBM.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188, etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

The MBM proproteins and tandem Fab MBMs of the disclosure can be formulated as pharmaceutical compositions comprising the MBM proproteins or tandem Fab MBMs, for example containing one or more pharmaceutically acceptable excipients or carriers. To prepare pharmaceutical or sterile compositions comprising the MBM proproteins and tandem Fab MBMs of the present disclosure, a MBM proprotein or tandem Fab MBM preparation can be combined with one or more pharmaceutically acceptable excipient or carrier.

For example, formulations of MBM proproteins and tandem Fab MBMs can be prepared by mixing MBM proproteins or tandem Fab MBMs with physiologically acceptable carriers, excipients, or stabilizers in the form of, e.g., lyophilized powders, slurries, aqueous solutions, lotions, or suspensions (see, e.g., Hardman et al., 2001, Goodman and Gilman's The Pharmacological Basis of Therapeutics, McGraw-Hill, New York, N.Y.; Gennaro, 2000, Remington: The Science and Practice of Pharmacy, Lippincott, Williams, and Wilkins, New York, N.Y.; Avis, et al. (eds.), 1993, Pharmaceutical Dosage Forms: General Medications, Marcel Dekker, NY; Lieberman, et al. (eds.), 1990, Pharmaceutical Dosage Forms: Tablets, Marcel Dekker, NY; Lieberman, et al. (eds.), 1990, Pharmaceutical Dosage Forms: Disperse Systems, Marcel Dekker, NY; Weiner and Kotkoskie, 2000, Excipient Toxicity and Safety, Marcel Dekker, Inc., New York, N.Y.).

An effective amount for a particular subject may vary depending on factors such as the condition being treated, the overall health of the subject, the method route and dose of administration and the severity of side effects (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

A composition of the present disclosure may also be administered via one or more routes of administration using one or more of a variety of methods known in the art. As will be appreciated by the skilled artisan, the route and/or mode of administration will vary depending upon the desired results. Selected routes of administration for MBM proproteins and tandem Fab MBMs include intravenous, intramuscular, intradermal, intraperitoneal, subcutaneous, spinal or other general routes of administration, for example by injection or infusion. General administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the disclosure can be administered via a non-general route, such as a topical, epidermal or mucosal route of administration, for example, intranasally, orally, vaginally, rectally, sublingually or topically. In one embodiment, the MBM proproteins and tandem Fab MBMs are administered by infusion. In another embodiment, the MBM proprotein or tandem Fab MBM of the disclosure is administered subcutaneously.

### 6.14. Therapeutic Indications and Methods of Treatment

The MBM proproteins and tandem Fab MBMs of the disclosure can be used in the treatment of any proliferative disorder (e.g., cancer) that expresses a TAA. In particular embodiments, the cancer is acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, or Wilms tumor.

Table K below shows exemplary indications for which MBM proproteins and tandem Fab MBMs targeting particular TAAs can be used.

TABLE K
Examples of Tumor-Associated Antigen Indications

| Target | Exemplary Indication(s) |
|---|---|
| ADRB3 | Ewing sarcoma |
| ALK | NSCLC, ALCL, IMT, neuroblastoma |
| B7H3 | melanoma, osteosarcoma, leukemia, breast, prostate, ovarian, pancreatic, |
| | colorectal cancers |
| BCMA | multiple myeloma, leukemia (e.g., acute lymphoblastic leukemia (“ALL”), |
| | acute myeloid leukemia (“AML”), chronic lymphocytic leukemia (“CLL”), |
| | chronic myeloid leukemia (“CML”) and hairy cell leukemia (“HCL”)); |
| | lymphoma (e.g., Hodgkin's lymphoma, non-Hodgkin's lymphoma, including |
| | diffuse large B-cell lymphoma (“DLBCL”)) |
| Cadherin 17 | gastric, pancreatic, and colorectal adenocarcinomas |
| CAIX | clear-cell renal cell carcinoma, hypoxic solid tumors, head and neck |
| | squamous carcinoma |
| CD123 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CD171 | neuroblastoma, paraganglioma |
| CD179a | B cell malignancies |
| CD19 | leukemia (e.g., ALL, CLL, AML, CML, HCL); lymphoma (e.g., Hodgkin's |
| | lymphoma, non-Hodgkin's lymphoma, e.g., DLBCL); multiple myeloma. |
| CD20 | leukemia (e.g., ALL, CLL, AML, CML, HCL); lymphoma (e.g., Hodgkin's |
| | lymphoma, non-Hodgkin's lymphoma, e.g., DLBCL); multiple myeloma. |
| CD22 | leukemia (e.g., ALL, CLL, AML, CML, HCL); lymphoma (e.g., Hodgkin's |
| | lymphoma, non-Hodgkin's lymphoma, e.g., DLBCL); multiple myeloma; |
| | lung cancer |
| CD24 | ovarian, breast, prostate, bladder, renal, non-small cell carcinomas |
| CD30 | anaplastic large cell lymphoma, embryonal carcinoma, Hodgkin Lymphoma |
| CD32b | B cell malignancies, gastric, pancreatic, esophageal, glioblastoma, breast, |
| | colorectal |
| CD33 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CD38 | leukemia (e.g., ALL, CLL, AML, CML, HCL); lymphoma (e.g., Hodgkin's |
| | lymphoma, non-Hodgkin's lymphoma, e.g., DLBCL); multiple myeloma |
| CD44v6 | colon cancer, head and neck small cell carcinoma |
| CD97 | B cell malignancies, gastric, pancreatic, esophageal, glioblastoma, breast, |
| | colorectal |
| CEA | colorectal carcinoma, gastric carcinoma, pancreatic carcinoma, lung |
| (CEACAM5) | cancer, breast cancer, medullary thyroid carcinoma |
| CLDN6 | ovarian, breast, lung cancer |
| CLL-1 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CS1 (SLAMF7) | multiple myeloma |
| EGFR | squamous cell carcinoma of lung, anal cancer, glioblastoma, epithelial |
| | tumors of head and neck, colon cancer |
| EGFRvIII | Glioblastoma |
| EPCAM | gastrointestestinal carcinoma, colorectal cancer |
| EphA2 | kaposi's sarcoma, glioblastoma, solid tumors, glioma |
| Ephrin B2 | thyroid cancer, breast cancer, malignant melanoma |
| ERBB2 | breast, ovarian, gastric cancers, lung adenocarcinoma, non-small cell lung |
| (Her2/neu) | cancer, uterine cancer, uterine serous endometrial carcinoma, salivary duct |
| | carcinoma |
| FAP | pancreatic cancer, colorectal cancer, metastasis, epithelial cancers, soft |
| | tissue sarcomas |
| FCRL5 | multiple myeloma |
| FLT3 | leukemia (e.g., ALL, CLL, AML, CML, HCL), lymphoma (e.g., Hodgkin's |
| | lymphoma, non-Hodgkin's lymphoma, e.g., DLBCL), multiple myeloma |
| Folate receptor | ovarian, breast, renal, lung, colorectal, brain cancers |
| alpha | |
| Folate receptor | ovarian cancer |
| beta | |
| Fucosyl GM1 | AML, myeloma |
| GD2 | malignant melanoma, neuroblastoma |
| GD3 | Melanoma |
| GloboH | ovarian, gastric, prostate, lung, breast, and pancreatic cancers |
| gp100 | Melanoma |
| GPNMB | breast cancer, head and neck cancers |
| GPR20 | GIST |
| GPR64 | Ewing sarcoma, prostate, kidney and lung sarcomas |
| GPRC5D | multiple myeloma |
| HAVCR1 | renal cancer |
| HER2 | HER-2 (+) adenocarcinoma of gastroesophageal junction, HER-2 positive |
| | gastric adenocarcinoma, HER2 positive carcinoma of breast |
| HER3 | colon and gastric cancers |
| HMWMAA | melanoma, glioblastoma, breast cancer |
| IGF-I receptor | breast, prostate, lung cancers |
| IL11Rα | papillary thyroid cancer, osteosarcoma, colorectal adenocarcinoma, |
| | lymphocytic leukemia |
| IL13Rα2 | renal cell carcinoma, prostate cancer, gliomas, head and neck cancer, |
| | astrocytoma |
| KIT | myeloid leukemia, kaposi's sarcoma, erythroleukemia, gastrointestinal |
| | stromal tumors |
| KLRG2 | breast cancers, lung cancers and ovarian cancers. |
| LewisY | squamous cell lung carcinoma, lung adenocarcinoma, ovarian carcinoma, |
| | and colorectal adenocarcinoma |
| LMP2 | prostate cancer, Hodgkin's lymphoma, nasopharyngeal carcinoma |
| LRP6 | breast cancer |
| LY6K | breast, lung, ovarian, and cervical cancer |
| LYPD8 | colorectal and gastric cancers |
| Mesothelin | mesothelioma, pancreatic cancer, ovarian cancer, stomach cancer, lung |
| | cancer, endometrial cancer |
| MUC1 | breast and ovarian cancers, lung, stomach, pancreatic, prostate cancers |
| NCAM | melanoma, Wilms' tumor, small cell lung cancer, neuroblastoma, myeloma, |
| | paraganglioma, pancreatic acinar cell carcinoma, myeloid leukemia |
| NY-BR-1 | breast cancer |
| o-acetyl GD2 | neuroblastoma, melanoma |
| OR51E2 | prostate cancer |
| PANX3 | Osteosarcoma |
| PLAC1 | hepatocellular carcinoma |
| Polysialic acid | small cell lung cancer |
| PDGFR-beta | myelomonocytic leukemia, chronic myeloid leukemia, acute myelogenous |
| | leukemia, acute lymphoblastic leukemia |
| PRSS21 | colon cancer, testicular cancer, ovarian cancer |
| PSCA | prostate cancer, gastric and bladder cancers |
| PSMA | prostate cancer |
| ROR1 | metastatic cancers, chronic lymphocytic leukemia, solid tumors in lung, |
| | breast, ovarian, colon, pancreatic, sarcoma |
| SLC34A2 | bladder cancer |
| SLC39A6 | breast cancer, esophageal cancer |
| SLITRK6 | breast cancer, urothelial cancer, lung cancer |
| SSEA-4 | breast cancer, cancer stem cells, epithelial ovarian carcinoma |
| STEAP1 | prostate cancer |
| STEAP2 | prostate cancer (including castrate-resistant prostate cancer), bladder |
| | cancer, cervical cancer, lung cancer, colon cancer, kidney cancer, breast |
| | cancer, pancreatic cancer, stomach cancer, uterine cancer, ovarian |
| | cancer, preferably prostate cancer |
| TACSTD2 | carcinomas, e.g., non-small-cell lung cancer |
| TAG72 | ovarian, breast, colon, lung, pancreatic cancers, gastric cancer |
| TEM1/CD248 | colorectal cancer |
| TEM7R | colorectal cancer |
| Tn | colorectal, breast cancers, cervical, lung, stomach cancers |
| TSHR | thyroid cancer, multiple myeloma |
| Tyrosinase | prostate cancer, melanoma |
| UPK2 | bladder cancer |
| VEGFR2 | ovarian and pancreatic cancers, renal cell carcinoma, colorectal cancer, |
| | medullary thyroid carcinoma |

Additional TAAs and corresponding indications are disclosed in, e.g., Hafeez et al., 2020, Molecules 25:4764, doi: 10.3390/molecules25204764, particularly in Table 1. Table 1 is incorporated by reference in its entirety here.

### 7. NUMBERED EMBODIMENTS

While various specific embodiments have been illustrated and described, it will be appreciated that various changes can be made without departing from the spirit and scope of the disclosure(s). The present disclosure is exemplified by the numbered embodiments set forth below.

In the numbered embodiments that follow, the TAA ABS preferably binds to a mammalian TAA, the TCE ABS preferably binds to a mammalian TCE, the Fc domains are preferably derived from a mammalian antibody, and the subjects preferably mammals. More preferably, the mammal is human.

The present disclosure is exemplified by the Group A and Group B numbered embodiments set forth below. Unless otherwise specified, features of any of the numbered embodiments of a particular group are applicable mutatis mutandis to the numbered embodiments of the other groups.

### Group A Numbered Embodiments

1. A binding molecule comprising:
(a) a first Fc domain;
(b) a second Fc domain; and
(c) a first component of a first antigen-binding site (ABS) connected to the first Fc domain via a protease-cleavable linker (PCL),
wherein (i) the PCL is 25 or fewer amino acids in length, and/or (ii) the first ABS is sterically hindered from binding its target.

2. The binding molecule of embodiment 1, wherein the binding of the first ABS to its target is enhanced following protease cleavage of the PCL.

3. The binding molecule of embodiment 1, wherein the first ABS is sterically hindered from binding its target prior to protease cleavage of the PCL and the steric hindrance is released following protease cleavage of the PCL.

4. The binding molecule of any one of embodiments 1 to 3, wherein the PCL is 20 or fewer amino acids in length.

5. The binding molecule of any one of embodiments 1 to 3, wherein the PCL is 15 or fewer amino acids in length.

6. The binding molecule of any one of embodiments 1 to 3, wherein the PCL is 10 or fewer amino acids in length.

7. The binding molecule of any one of embodiments 1 to 6, wherein, following protease cleavage of the PCL, the first ABS has enhanced binding to its target by at least 10-fold.

8. The binding molecule of any one of embodiments 1 to 6, wherein, following protease cleavage of the PCL, the first ABS has enhanced binding to its target by at least 100-fold.

9. The binding molecule of any one of embodiments 1 to 8, wherein the first ABS is a Fab.

10. The binding molecule of any one of embodiments 1 to 8, wherein the first ABS is a Fv.

11. The binding molecule of any one of embodiments 1 to 10, wherein the first component is a VH.

12. The binding molecule of any one of embodiments 1 to 10, wherein the first component is a VL.

13. The binding molecule of any one of embodiments 1 to 12, wherein the binding molecule comprises at least two polypeptide chains, where the first polypeptide chain comprises the first Fc domain and the first component of the first ABS and the second polypeptide chain comprises the second Fc domain.

14. The binding molecule of embodiment 13, wherein the second polypeptide chain comprises a second component of the first ABS.

15. The binding molecule of embodiment 14, wherein the first component and the second component associate to form the first ABS.

16. The binding molecule of embodiment 14 or 15, wherein the first component is a VH and the second component is a VL.

17. The binding molecule of embodiment 16, wherein (1) the first polypeptide chain further comprises a CH1 C-terminal to the VH, and (2) the second polypeptide chain further comprises a CL C-terminal to the VL.

18. The binding molecule of any one of embodiments 13 to 15, which further comprises a third polypeptide chain comprising a third component of the first ABS.

19. The binding molecule of any one of embodiments 1 to 18, which is a multispecific binding molecule proprotein comprising a second ABS.

20. The binding molecule of embodiment 19, wherein (a) the first ABS is a TCE ABS and the second ABS is a TAA ABS or (b) the first ABS is a TAA ABS and the second ABS is a TCE ABS.

21. The binding molecule of embodiment 20, wherein the first ABS is the TCE ABS.

22. The binding molecule of embodiment 21, wherein the TCE ABS is capable of binding to CD3, TCRαβ, or TCRγδ.

23. The binding molecule any one of embodiments 20 to 22, wherein the TAA ABS is capable of binding to:
(a) any TAA identified in Section 6.6; or
(b) AFP, ALK, a BAGE protein, BIRC5 (survivin), BIRC7, β-catenin, brc-abl, BRCA1, BORIS, CA9, carbonic anhydrase IX, caspase-8, CALR, CEACAM5 (also known as carcinoembryonic antigen or CEA), CCR5, CD19, CD20 (MS4A1), CD22, CD30, CD40, CDK4, CEA, CTLA4, cyclin-B1, CYP1B1, EGFR, EGFRvIII, ErbB2/Her2, ErbB3, ErbB4, ETV6-AML, EpCAM, EphA2, Fra-1, FOLR1, a GAGE protein (e.g., GAGE-1 or -2), GD2, GD3, GloboH, glypican-3, GM3, gp100, Her2, HLA/B-raf, HLA/k-ras, HLA/MAGE-A3, hTERT, LMP2, MAGE proteins (e.g., MAGE-1, -2, -3, -4, -6, and -12), MART-1, mesothelin, ML-IAP, Muc1, Muc2, Muc3, Muc4, Muc5, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, Ras, RGS5, Rho, SART-1, SART-3, STEAP1, STEAP2, TAG-72, TGF-β, TMPRSS2, Thompson-nouvelle antigen (Tn), TRP-1, TRP-2, tyrosinase, or uroplakin-3.

24. The binding molecule of any one of embodiments 1 to 23, wherein the first Fc domain and the second Fc domain associate to form a Fc heterodimer.

25. The binding molecule of embodiment 24, wherein the Fc heterodimer comprises knob-in-hole mutations, e.g., wherein (i) the first Fc domain comprises one or more knob mutations and the second Fc domain comprises one or more hole mutations or (ii) the first Fc domain comprises one or more hole mutations and the second Fc domain comprises one or more knob mutations.

26. The binding molecule of any one of embodiments 1 to 25, wherein the first Fc domain and/or the second Fc domain comprise a star mutation.

27. The binding molecule of any one of embodiments 1 to 26, wherein a tandem Fab is produced following protease cleavage of the PCL.

28. The binding molecule of any one of embodiments 1 to 26, wherein a multispecific binding molecule comprising the first Fc domain and the second Fc domain is produced following protease cleavage of the PCL.

29. The binding molecule of any one of embodiments 1 to 28, wherein the PCL comprises a substrate sequence cleavable by any protease set forth in Table A.

30. The binding molecule of any one of embodiments 1 to 29, wherein the PCL comprises one or more substrate sequences selected from the substrate sequences set forth in Table B.

31. The binding molecule of any one of embodiments 1 to 30, wherein the PCL comprises the amino acid sequence of any of the PCL sequences selected from the sequences set forth in Table D.

32. The binding molecule of any one of embodiments 1 to 31, wherein the binding molecule has the configuration depicted in FIG. 3A.

33. The binding molecule of any one of embodiments 1 to 31, wherein the binding molecule has the configuration depicted in FIG. 3B.

34. The binding molecule of any one of embodiments 1 to 31, wherein the binding molecule has the configuration depicted in FIG. 3C.

35. The binding molecule of any one of embodiments 1 to 31, wherein the binding molecule has the configuration depicted in FIG. 3D.

36. The binding molecule of any one of embodiments 1 to 31, wherein the binding molecule has the configuration depicted in FIG. 3E.

37. The binding molecule of any one of embodiments 1 to 31, wherein the binding molecule has the configuration depicted in FIG. 3F.

38. The binding molecule of any one of embodiments 1 to 31, wherein the binding molecule has the configuration depicted in FIG. 7.

39. The binding molecule of any one of embodiments 1 to 38, wherein the first Fc domain and/or the second Fc domain have at least about 90% sequence identity to any one of SEQ ID NOs: 335, 336, 337, and 338.

40. The binding molecule of any one of embodiments 1 to 38, wherein the first Fc domain and/or the second Fc domain have at least about 95% sequence identity to any one of SEQ ID NOs: 335, 336, 337, and 338.

41. The binding molecule of any one of embodiments 1 to 38, wherein the first Fc domain and/or the second Fc domain have at least about 95% sequence identity to any one of SEQ ID NOs: 335, 336, 337, and 338.

42. The binding molecule of any one of embodiments 1 to 41, wherein the first Fc domain and/or the second Fc domain comprise one or more amino acid substitutions that reduce effector function (e.g., as described in Section 6.10.1).

43. The binding molecule of any one of embodiments 1 to 41, wherein the first Fc domain and/or the second Fc domain comprise one or more amino acid substitutions that promote heterodimerization (e.g., as described in Section 6.10.2).

44. A pharmaceutical composition comprising the binding molecule of any one of embodiments 1 to 43 and an excipient.

45. A method of treating cancer, comprising administering to a subject suffering from cancer an effective amount of the binding molecule of any one of embodiments 1 to 43 or the pharmaceutical composition of embodiment 44.

46. The method of embodiment 45, in which the cancer is associated with expression of an epitope bound by the TAA ABS, e.g., as set forth in Table K.

47. A nucleic acid or plurality of nucleic acids encoding the binding molecule of any one of embodiments 1 to 43.

48. A cell transfected with one or more expression vectors comprising one or more nucleic acid sequences encoding the binding molecule of any one of embodiments 1 to 43 under the control of one or more promoters.

49. A method of producing a binding molecule, comprising:
(a) culturing the cell of embodiment 48 in conditions under which the binding molecule is expressed; and
(b) recovering the binding molecule from the cell culture.

50. The method of embodiment 49, which further comprises enriching for the binding molecule and/or purifying the binding molecule.

### Group B Numbered Embodiments

1. A multispecific binding molecule (MBM) proprotein, comprising
(a) a first Fc domain and a second Fc domain capable of associating to form an Fc region;
(b) a T-cell engaging antigen-binding site (“TCE ABS”) C-terminal to the first Fc domain and/or the second Fc domain;
(c) at least one antigen-binding site that is capable of binding to a tumor-associated antigen (“TAA ABS”); and
(d) a protease-cleavable linker (PCL) C-terminal to the first Fc domain or the second Fc domain,
   wherein the TAA ABS in the MBM proprotein is capable of binding to its target when the PCL is in the uncleaved state and the binding of the TCE ABS to its target is enhanced following protease cleavage of the PCL.

2. The MBM proprotein of embodiment 1, wherein cleavage of the PCL releases an MBM comprising the TAA ABS and the TCE ABS.

3. The MBM proprotein of embodiment 1 or embodiment 2, wherein the TCE ABS in the MBM proprotein is masked by an anti-idiotypic antibody.

4. The MBM proprotein of embodiment 1 or embodiment 2, wherein the TCE ABS in the MBM proprotein is sterically hindered from binding to its target by the Fc domain.

5. The MBM proprotein of any one of embodiments 1 to 4, wherein the MBM comprises the first and second Fc domains.

6. The MBM proprotein of embodiment 5, which has the configuration depicted in FIG. 1A.

7. The MBM proprotein of embodiment 5, which has the configuration depicted in FIG. 1B.

8. The MBM proprotein of embodiment 5, which has the configuration depicted in FIG. 1C.

9. The MBM proprotein of embodiment 5, which has the configuration depicted in FIG. 3A.

10. The MBM proprotein of embodiment 5, which has the configuration depicted in FIG. 3B.

11. The MBM proprotein of embodiment 5, which has the configuration depicted in FIG. 3C.

12. The MBM proprotein of embodiment 5, which has the configuration depicted in FIG. 3D.

13. The MBM proprotein of embodiment 5, which has the configuration depicted in FIG. 3E.

14. The MBM proprotein of embodiment 5, which has the configuration depicted in FIG. 3F.

15. The MBM proprotein of any one of embodiments 1 to 4, wherein the MBM lacks the first and second Fc domains.

16. The MBM proprotein of embodiment 15, which has the configuration depicted in FIG. 5.

17. The MBM proprotein of embodiment 15, which has the configuration depicted in FIG. 7.

18. A multispecific binding molecule (MBM) proprotein, which is optionally an MBM of any one of embodiments 1 to 8, comprising:
(a) a first polypeptide chain comprising, in an N- to C-terminal orientation:
   (i) an optional first linker;
   (ii) a first Fc domain;
   (iii) a second linker; and
   (iv) a T-cell engaging antigen-binding site (“TCE ABS”) or component thereof; and
(b) a second polypeptide chain comprising, in an N- to C-terminal orientation:
   (i) an optional third linker;
   (ii) a second Fc domain capable of heterodimerizing with the first Fc domain to form an Fc heterodimer;
   (iii) a fourth linker which is a protease-cleavable linker (“PCL”); and
   (iv) an antigen-binding site which is an anti-idiotype of the TCE ABS (“anti-TCE ABS”) or component thereof,
      wherein the first polypeptide chain and/or second polypeptide chain comprises an antigen-binding site that is capable of binding to a tumor-associated antigen (“TAA ABS”) or component thereof.

19. The MBM proprotein of embodiment 18, wherein the TAA ABS(s), TCE ABS and anti-TCE ABS are in the form of Fabs and wherein the TAA ABS chain(s), the TCE ABS chain and anti-TCE ABS chain comprise VH domains associated with their respective VL domains on separate polypeptide chains.

20. The MBM proprotein of embodiment 18, wherein the VL domains are universal light chain VL domains.

21. The MBM proprotein of any one of embodiments 1 to 18, wherein the anti-TCE ABS is in the form of an scFv.

22. The MBM proprotein of any one of embodiments 1 to 18, wherein the anti-TCE ABS is in the form of a VHH.

23. The MBM proprotein of any one of embodiments 1 to 18, wherein the anti-TCE ABS is in the form of a VH.

24. The MBM proprotein of any one of embodiments 18 to 20, wherein the linkers other than the PCL are non-cleavable linkers (“NCLs”).

25. A multispecific binding molecule (MBM) proprotein which is optionally an MBM of any one of embodiments 1 to 5 and 9 to 14, comprising:
(a) a first polypeptide chain comprising, in an N- to C-terminal orientation:
   (i) an optional first linker;
   (ii) a first Fc domain;
   (iii) a second linker; and
   (iv) a T-cell engaging antigen binding site (“TCE ABS”) component comprising a VH;
(b) a second polypeptide chain comprising, in an N- to C-terminal orientation:
   (i) an optional third linker;
   (ii) a second Fc domain capable of heterodimerizing with the first Fc domain to form an Fc heterodimer;
   (iii) a fourth linker; and
   (iv) a T-cell engaging antigen binding site (“TCE ABS”) component comprising a VL,
      wherein (A) the second linker or fourth linker is a protease-cleavable linker (“PCL”), (B) the first polypeptide chain and/or second polypeptide chain comprises an antigen-binding site that is capable of binding to a tumor-associated antigen (“TAA ABS”), and (C) the TCE ABS is sterically hindered from binding to its target.

26. The MBM proprotein of embodiment 25, wherein the TCE ABS is a Fab.

27. The MBM proprotein of embodiment 26, wherein the first polypeptide chain comprises a CH1 domain C-terminal to the VH of the TCE ABS and the second polypeptide chain comprises a CL domain C-terminal to the VL of the TCE ABS.

28. The MBM proprotein of embodiment 25, wherein the TCE ABS is an Fv.

29. The MBM proprotein of embodiment 28, wherein the first polypeptide chain lacks a CH1 domain C-terminal to the VH of the TCE ABS and the second polypeptide chain lacks a CL domain C-terminal to the VL of the TCE ABS.

30. The MBM proprotein of any one of embodiments 25 to 29, wherein the TAA ABS(s) is (are) in the form of a Fabs and wherein the TAA ABS chain(s) each comprises a VH domain associated with it respective VL domain on a separate polypeptide chain.

31. The MBM proprotein of embodiment 30, wherein the VL domain on a separate polypeptide chain is universal light chain VL domain.

32. The MBM proprotein of any one of embodiments 25 to 31, wherein the linkers other than the PCL are non-cleavable linkers (“NCLs”).

33. The MBM proprotein of any one of embodiments 25 to 32, wherein the second linker or fourth linker are 25 or fewer amino acids, 20 or fewer amino acids, 15 or fewer amino acids, or 10 or fewer amino acids.

34. A multispecific binding molecule (MBM) proprotein, which is optionally an MBM of any one of embodiments 1 to 8, 15, and 16, comprising:
(a) a first polypeptide chain comprising, in an N- to C-terminal orientation:
   (i) an optional first linker;
   (ii) a first Fc domain;
   (iii) a second linker; and
   (iv) an antigen-binding site which is an anti-idiotype of the TCE ABS (“anti-TCE ABS”) of the second polypeptide chain or component thereof; and
(b) a second polypeptide chain comprising, in an N- to C-terminal orientation:
   (i) an optional third linker;
   (ii) a second Fc domain capable of heterodimerizing with the first Fc domain to form an Fc heterodimer;
   (iii) a fourth linker which is a protease-cleavable linker (“PCL”);
   (iv) a T-cell engaging antigen-binding site (“TCE ABS”) or component thereof;
   (v) an optional fifth linker; and
   (vi) an antigen-binding site that is capable of binding to a tumor-associated antigen (“TAA ABS”) or component thereof.

35. The MBM proprotein of embodiment 34, wherein the TAA ABS, TCE ABS and anti-TCE ABS are in the form of Fabs and wherein the TAA ABS chain, the TCE ABS chain and anti-TCE ABS chain comprise VH domains associated with their respective VL domains on separate polypeptide chains.

36. The MBM proprotein of embodiment 35, wherein the VL domains are universal light chain VL domains.

37. The MBM proprotein of embodiment 34, wherein the anti-TCE ABS is in the form of an scFv.

38. The MBM proprotein of embodiment 34, wherein the anti-TCE ABS is in the form of a VHH.

39. The MBM proprotein of embodiment 34, wherein the anti-TCE ABS is in the form of a VH.

40. The MBM proprotein of any one of embodiments 34 to 39, wherein the linkers other than the PCL are non-cleavable linkers (“NCLs”).

41. A multispecific binding molecule (MBM) proprotein which is optionally an MBM of any one of embodiments 1 to 4, 15 and 17, comprising:
(a) a first polypeptide chain comprising, in an N- to C-terminal orientation:
   (i) an optional first linker;
   (ii) a first Fc domain;
   (iii) a second linker which is a protease cleavable linker (“PCL”); and
   (iv) a T-cell engaging antigen binding site (“TCE ABS”) component comprising a VH;
(b) a second polypeptide chain comprising, in an N- to C-terminal orientation:
   (i) an optional third linker;
   (ii) a second Fc domain capable of heterodimerizing with the first Fc domain to form an Fc heterodimer;
   (iii) a fourth linker which is a protease cleavable linker (“PCL”); and
   (iv) a T-cell engaging antigen binding site (“TCE ABS”) component comprising a VL,
      wherein the first polypeptide chain or second polypeptide chain comprises an antigen-binding site that is capable of binding to a tumor-associated antigen (“TAA ABS”) or a component thereof C-terminal to the TCE ABS chain.

42. The MBM proprotein of embodiment 41, wherein the TCE ABS is a Fab.

43. The MBM proprotein of embodiment 42, wherein the first polypeptide chain comprises a CH1 domain C-terminal to the VH of the TCE ABS and the second polypeptide chain comprises a CL domain C-terminal to the VL of the TCE ABS.

44. The MBM proprotein of embodiment 41, wherein the TCE ABS is an Fv.

45. The MBM proprotein of embodiment 44, wherein the first polypeptide chain lacks a CH1 domain C-terminal to the VH of the TCE ABS and the second polypeptide chain lacks a CL domain C-terminal to the VL of the TCE ABS.

46. The MBM proprotein of any one of embodiments 41 to 45, wherein the TAA ABS is in the form of a Fab and wherein the TAA ABS chain comprises a VH domain associated with a VL domain on a separate polypeptide chain.

47. The MBM proprotein of embodiment 46, wherein the VL domain on a separate polypeptide chain is universal light chain VL domain.

48. The MBM proprotein of any one of embodiments 41 to 47, wherein the linkers other than the PCLs are non-cleavable linkers (“NCLs”).

49. The MBM proprotein of any one of embodiments 41 to 48, wherein the second linker or fourth linker is 25 or fewer amino acids, 20 or fewer amino acids, 15 or fewer amino acids, or 10 or fewer amino acids.

50. The MBM proprotein of any one of embodiments 1 to 49, wherein each PCL comprises a substrate sequence cleavable by any protease set forth in Table A.

51. The MBM proprotein of any one of embodiments 1 to 50, wherein each PCL comprises one or more substrate sequences selected from the substrate sequences set forth in Table B.

52. The MBM proprotein of any one of embodiments 1 to 51, wherein each PCL comprises one or more spacer sequences selected from the substrate sequences set forth in Table C, optionally wherein the PCL comprises or consists of any one of the following amino acid sequences:

(a)
(SEQ ID NO: 205)
GGGGSGGGGSGGGGSISSGLLSGRSDNHGGSGGS;
(b)
(SEQ ID NO: 206)
GGGGSGGGGSGGGGSVPLSLYSGGGSGGSGGSGS;
(c)
(SEQ ID NO: 230)
ISSGLLSGRSDNH;

and
(d) a variant of any one of the foregoing with 1-5 amino acid substitutions.

53. The MBM proprotein of any one of embodiments 1 to 52, wherein each PCL comprises the amino acid sequence of any of the PCL sequences selected from the substrate sequences set forth in Table D.

54. The MBM proprotein of any one of embodiments 1 to 53, wherein all linkers in the molecule other than the PCL(s) are non-cleavable linkers (“NCLs”).

55. The MBM proprotein of embodiment 54, wherein the NCLs are selected from Table E.

56. The MBM proprotein of any one of embodiments 1 to 55, wherein the TAA ABS is capable of binding to:
(a) any TAA identified in Section 4.5; or
(b) AFP, ALK, a BAGE protein, BIRC5 (survivin), BIRC7, β-catenin, brc-abl, BRCA1, BORIS, CA9, carbonic anhydrase IX, caspase-8, CALR, CEACAM5 (also known as carcinoembryonic antigen or CEA), CCR5, CD19, CD20 (MS4A1), CD22, CD30, CD40, CDK4, CEA, CTLA4, cyclin-B1, CYP1B1, EGFR, EGFRvIII, ErbB2/Her2, ErbB3, ErbB4, ETV6-AML, EpCAM, EphA2, Fra-1, FOLR1, a GAGE protein (e.g., GAGE-1 or -2), GD2, GD3, GloboH, glypican-3, GM3, gp100, Her2, HLA/B-raf, HLA/k-ras, HLA/MAGE-A3, hTERT, LMP2, MAGE proteins (e.g., MAGE-1, -2, -3, -4, -6, and -12), MART-1, mesothelin, ML-IAP, Muc1, Muc2, Muc3, Muc4, Muc5, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, Ras, RGS5, Rho, SART-1, SART-3, STEAP1, STEAP2, TAG-72, TGF-β, TMPRSS2, Thompson-nouvelle antigen (Tn), TRP-1, TRP-2, tyrosinase, or uroplakin-3.

57. The MBM proprotein of any one of embodiments 1 to 56, wherein the TAA ABS (a) comprises the (i) CDR or (ii) VH and VL sequences of antibody set forth in Table F or (b) competes with the antibody set forth in Table F for binding to the TAA.

58. The MBM proprotein of any one of embodiments 1 to 57, wherein the TAA is CEACAM5.

59. The MBM proprotein of any one of embodiments 1 to 57, wherein the TAA is EpCAM.

60. The MBM proprotein of any one of embodiments 1 to 57, wherein the TAA is HER2.

61. The MBM proprotein of any one of embodiments 1 to 5571, wherein the TAA is PMSA.

62. The MBM proprotein of any one of embodiments 1 to 57, wherein the TAA is STEAP2.

63. The MBM proprotein of any one of embodiments 1 to 57, wherein the TAA is EGFR.

64. The MBM proprotein of any one of embodiments 1 to 57, wherein the TCE ABS is capable of binding to a component of the T-cell receptor (TCR) complex.

65. The MBM proprotein of embodiment 64, wherein the component of the TCR complex is CD3.

66. The MBM proprotein of embodiment 64, wherein the component of the TCR complex is TCRαβ.

67. The MBM proprotein of embodiment 64, wherein the component of the TCR complex is TCR γδ.

68. The MBM proprotein of any one of embodiments 1 to 67, wherein the TCE ABS (a) comprises the (i) CDR or (ii) VH and VL sequences of antibody set forth in Table G or (b) competes with the antibody set forth in Table G for binding to its target.

69. The MBM proprotein of any one of embodiments 1 to 68, which produces a bispecific binding molecule (“BBM”) following protease cleavage of PCL.

70. The MBM proprotein of embodiment 69, wherein the BBM is bivalent.

71. The MBM proprotein of embodiment 69, wherein the BBM is trivalent.

72. The MBM proprotein of any one of embodiments 1 to 71, wherein the Fc region is heterodimeric.

73. The MBM proprotein of embodiment 72, wherein the Fc heterodimer comprises knob-in-hole mutations.

74. The MBM proprotein of any one of embodiments 1 to 72, wherein at least one Fc domain comprises a star mutation as compared to a wild type Fc domain.

75. The MBM proprotein of any one of embodiments 1 to 74, wherein the TCE ABS comprises a VL having a deletion of one or more amino acids at the N-terminus.

76. The MBM proprotein of any one of embodiments 1 to 74, wherein the TCE ABS comprises a VH having a deletion of one or more amino acids at the N-terminus.

77. The MBM proprotein of any one of embodiments 1 to 76, wherein the first Fc domain comprises a deletion of one or more amino acids at the C-terminus relative to a wild type Fc domain.

78. The MBM proprotein of embodiment 77, wherein the first Fc domain comprises a deletion of 1 amino acid at the C-terminus relative to a wild type Fc domain.

79. The MBM proprotein of embodiment 77, wherein the first Fc domain comprises a deletion of 2 amino acids at the C-terminus relative to a wild type Fc domain.

80. The MBM proprotein of embodiment 77, wherein the first Fc domain comprises a deletion of 3 amino acids at the C-terminus relative to a wild type Fc domain.

81. The MBM proprotein of embodiment 77, wherein the first Fc domain comprises a deletion of 4 amino acids at the C-terminus relative to a wild type Fc domain.

82. The MBM proprotein of embodiment 77, wherein the first Fc domain comprises a deletion of 5 amino acids at the C-terminus relative to a wild type Fc domain.

83. The MBM proprotein of embodiment 77, wherein the first Fc domain comprises a deletion of 6 amino acids at the C-terminus relative to a wild type Fc domain.

84. The MBM proprotein of any one of embodiments 1 to 83, wherein the second Fc domain comprises a deletion of one or more amino acids at the C-terminus relative to a wild type Fc domain.

85. The MBM proprotein of embodiment 84, wherein the second Fc domain comprises a deletion of 1 amino acid at the C-terminus relative to a wild type Fc domain.

86. The MBM proprotein of embodiment 84, wherein the second Fc domain comprises a deletion of 2 amino acids at the C-terminus relative to a wild type Fc domain.

87. The MBM proprotein of embodiment 84, wherein the second Fc domain comprises a deletion of 3 amino acids at the C-terminus relative to a wild type Fc domain.

88. The MBM proprotein of embodiment 84, wherein the second Fc domain comprises a deletion of 4 amino acids at the C-terminus relative to a wild type Fc domain.

89. The MBM proprotein of embodiment 84, wherein the second Fc domain comprises a deletion of 5 amino acids at the C-terminus relative to a wild type Fc domain.

90. The MBM proprotein of embodiment 84, wherein the second Fc domain comprises a deletion of 6 amino acids at the C-terminus relative to a wild type Fc domain.

91. A tandem Fab MBM comprising (1) a first polypeptide chain comprising from N- to C-terminal orientation a VH of a TCE ABS operably linked to a CH1 domain; an optional linker, e.g., non-cleavable linker; and a VH of a TAA ABS operably linked to a CH1 domain; (2) a second polypeptide chain comprising a VL of the TAA ABS operably linked to a CL domain; and (3) a third polypeptide chain comprising a VL of the TCE ABS operably linked to a CL domain.

92. A tandem Fab MBM comprising (1) a first polypeptide chain comprising from N- to C-terminal orientation (a) a VH of a TCE ABS; (b) a CH1 domain; (c) a non-cleavable linker; (d) a VH of a TAA ABS; and (c) a CH1 domain; (2) a second polypeptide chain comprising (a) a VL of the TAA ABS and (b) a CL domain; and (3) a third polypeptide chain comprising (a) a VL of the TCE ABS and (b) a CL domain.

93. A tandem Fab MBM comprising tandem Fab MBM comprising (1) a first polypeptide chain comprising from N- to C-terminal orientation a VH of a TAA ABS operably linked to a CH1 domain; an optional linker, e.g., non-cleavable linker; and a VH of a TCE ABS operably linked to a CH1 domain; (2) a second polypeptide chain comprising a VL of the TAA ABS operably linked to a CL domain; and (3) a third polypeptide chain comprising a VL of the TCE ABS operably linked to a CL domain.

94. A tandem Fab MBM comprising (1) a first polypeptide chain comprising from N- to C-terminal orientation (a) a VH of a TAA ABS; (b) a CH1 domain; (c) a non-cleavable linker; (d) a VH of a TCE ABS; and (c) a CH1 domain; (2) a second polypeptide chain comprising (a) a VL of the TAA ABS and (b) a CL domain; and (3) a third polypeptide chain comprising (a) a VL of the TCE ABS and (b) a CL domain.

95. The tandem Fab MBM of any one of embodiments 91 to 94, wherein the CH1 of the TCE ABS and the CH1 of the TAA ABS are the same.

96. The tandem Fab MBM of any one of embodiments 91 to 95, wherein the CL of the TCE ABS and the CL of the TAA ABS are the same.

97. The tandem Fab MBM of any one of embodiments 91 to 9674, wherein the VL of the TAA ABS and the TCE ABS are the same and are universal light chain VLs.

98. The tandem Fab MBM of any one of embodiments 91 to 97, wherein the TAA ABS is as defined in any one of embodiments 50 to 57.

99. The tandem Fab MBM of any one of embodiments 91 to 98, wherein the TCE ABS is as defined in any one of embodiments 64 to 68.

100. A pharmaceutical composition comprising the MBM proprotein of any one of embodiments 1 to 90 or the tandem Fab MBM of any one of embodiments 91 to 99 and an excipient.

101. A method of treating cancer, comprising administering to a subject suffering from cancer an effective amount of the MBM proprotein of any one of embodiments 1 to 90, the tandem Fab MBM of any one of embodiments 91 to 99, or the pharmaceutical composition of embodiment 100.

102. The method of embodiment 101, in which the cancer is associated with expression of an epitope bound by the TAA ABS, e.g., as set forth in Table K.

103. A nucleic acid or plurality of nucleic acids encoding the MBM proprotein of any one of embodiments 1 to 90 or the tandem Fab MBM of any one of embodiments 91 to 99.

104. A cell engineered to express the MBM proprotein of any one of embodiments 1 to 90 or the tandem Fab MBM of any one of embodiments 91 to 99.

105. A cell transfected with one or more expression vectors comprising one or more nucleic acid sequences encoding the MBM proprotein of any one of embodiments 1 to 90 or the tandem Fab MBM of any one of embodiments 91 to 99 under the control of one or more promoters.

106. A method of producing a MBM proprotein or tandem Fab MBM, comprising:
(a) culturing the cell of embodiment 104 or embodiment 105 in conditions under which the MBM is expressed; and
(b) recovering the MBM proprotein or tandem Fab MBM, as applicable, from the cell culture

107. The method of embodiment 106, which further comprises enriching for the MBM proprotein or tandem Fab MBM, as applicable.

108. The method of embodiment 106 or embodiment 107, which further comprises purifying the MBM proprotein or tandem Fab MBM, as applicable.

### 8. EXAMPLES

### 8.1. Example 1: Effect of Linker Length on Fc-Masking of C-Terminal Antigen Binding Sites

Anti-TAA×CD3 bispecific antibodies were generated having the configuration depicted in FIG. 3B with different linker lengths (5, 15 and 25 amino acids) C-terminal to the Fc domains, except that (in reference to FIG. 3B) Linker B was a non-cleavable linker. The goal of the study was to determine the effect of the linker separating the Fc domain from the C-terminal ABS or ABS chain on ABS's ability to bind its target, in this case CD3, on JURKAT cells. A traditional bispecific anti-TAA×CD3 bispecific antibody was used as a control for CD3 binding.

Flow cytometry analysis was utilized to determine binding of TAA×CD3 bispecific antibodies to JURKAT cells, followed by detection with an allophycocyanin (APC)-labeled anti-human IgG antibody. Briefly, 1×10⁵ cells/well were incubated for 30 minutes at 4° C. with a serial dilution of anti-TAA×CD3 bispecific antibodies. After incubation, the cells were washed twice with cold PBS containing 1% filtered FBS and an APC-labeled anti-human secondary antibody was added to the cells and incubated for an additional 30 minutes. Wells containing secondary only were used as a control.

After incubation, cells were washed, re-suspended in 200 μL cold PBS containing 1% filtered FBS and analyzed by flow cytometry on a BD FACS Canto II.

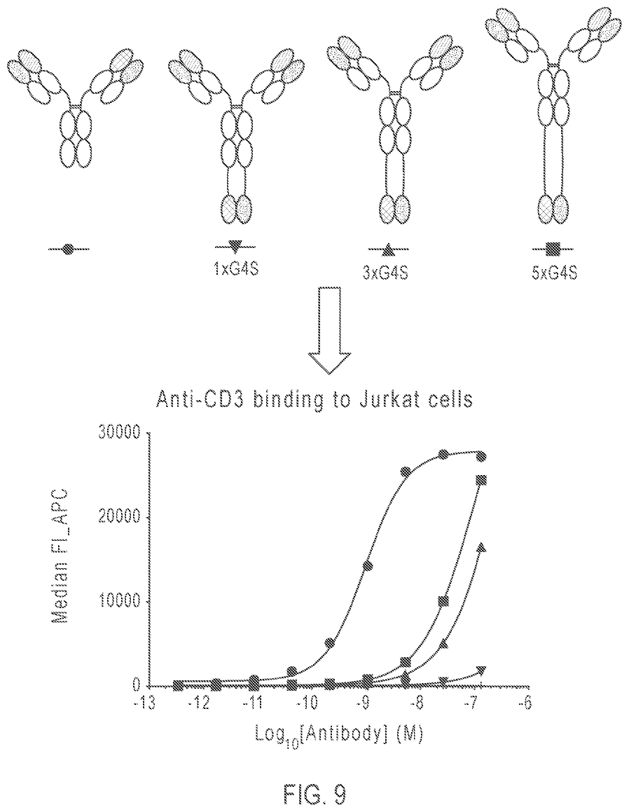

The results are shown in FIG. 9. As evident in FIG. 9, the ability of the CD3 ABS to bind its target correlated with linker length, with shorter linker lengths maximizing the steric hindrance by the Fc and resulting in the greatest inhibition of the CD3 binding, and longer linker lengths being more permissive for CD3 binding (but still reduced as compared to a traditional bispecific antibody).

### 8.2. Example 2: Fc Steric Hindrance Reduces Anti-TAA×CD3 Bispecific Antibody Target Binding

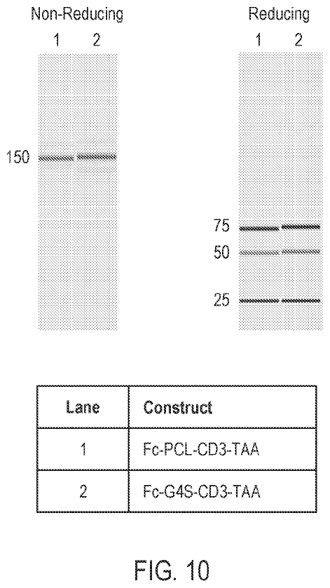

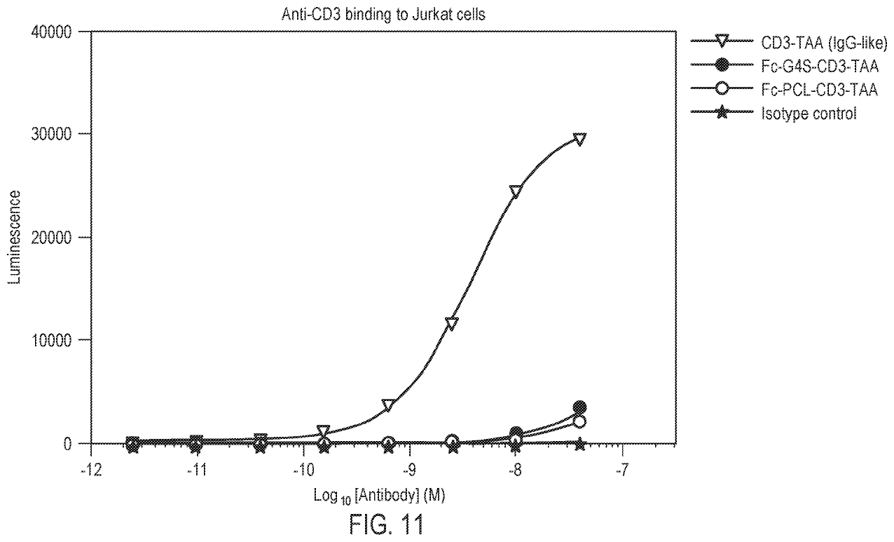

Anti-TAA×CD3 bispecific antibodies were generated having the configuration depicted in FIG. 7, wherein linkers A and B were either cleavable linkers as illustrated or replaced by noncleavable G4S linkers (SEQ ID NO: 1), by transient transfection in Expi293 cells. FIG. 10 shows the expression of an anti-TAA×CD3 bispecific antibody with cleavable linkers A and B (Fc-PCL-CD3-TAA) and an anti-TAA×CD3 bispecific antibody without cleavable linkers A and B (Fc-G4S-CD3-TAA) after single step Pro A purification.

Flow cytometry analysis was utilized as described in Section 8.1, whereby binding of TAA×CD3 bispecific antibodies to JURKAT cells was assessed.

Relative to the anti-TAA×CD3 bispecific antibody in IgG configuration, all anti-TAA×CD3 bispecific antibodies with Fc steric hindrance displayed inhibition of CD3 binding on JURKAT cells (FIG. 11A). Similar constructs with truncations at the C-terminus of the Fc by 4 or 6 amino acids and/or the N-terminus of the VH/VL by 1 or 2 amino acids similarly 5 inhibited binding of CD3 to JURKAT cells (data not shown).

### 9. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

1. A binding molecule comprising:
(a) a first Fc domain;
(b) a second Fc domain; and
(c) a first component of a first antigen-binding site (ABS) connected to the first Fc domain via a protease-cleavable linker (PCL),
wherein (i) the PCL is 25 or fewer amino acids in length, and/or (ii) the first ABS is sterically hindered from binding its target.

2. The binding molecule of claim 1, wherein the binding of the first ABS to its target is enhanced following protease cleavage of the PCL.

3. The binding molecule of claim 1, wherein the first ABS is sterically hindered from binding its target prior to protease cleavage of the PCL and the steric hindrance is released following protease cleavage of the PCL.

4. The binding molecule of any one of claims 1 to 3, wherein the PCL is 20 or fewer amino acids in length.

5. The binding molecule of any one of claims 1 to 3, wherein the PCL is 15 or fewer amino acids in length.

6. The binding molecule of any one of claims 1 to 3, wherein the PCL is 10 or fewer amino acids in length.

7. The binding molecule of any one of claims 1 to 6, wherein, following protease cleavage of the PCL, the first ABS has enhanced binding to its target by at least 10-fold.

8. The binding molecule of any one of claims 1 to 6, wherein, following protease cleavage of the PCL, the first ABS has enhanced binding to its target by at least 100-fold.

9. The binding molecule of any one of claims 1 to 8, wherein the first ABS is a Fab.

10. The binding molecule of any one of claims 1 to 8, wherein the first ABS is a Fv.

11. The binding molecule of any one of claims 1 to 10, wherein the first component is a VH.

12. The binding molecule of any one of claims 1 to 10, wherein the first component is a VL.

13. The binding molecule of any one of claims 1 to 12, wherein the binding molecule comprises at least two polypeptide chains, where the first polypeptide chain comprises the first Fc domain and the first component of the first ABS and the second polypeptide chain comprises the second Fc domain.

14. The binding molecule of claim 13, wherein the second polypeptide chain comprises a second component of the first ABS.

15. The binding molecule of claim 14, wherein the first component and the second component associate to form the first ABS.

16. The binding molecule of claim 14 or 15, wherein the first component is a VH and the second component is a VL.

17. The binding molecule of claim 16, wherein (1) the first polypeptide chain further comprises a CH1 C-terminal to the VH, and (2) the second polypeptide chain further comprises a CL C-terminal to the VL.

18. The binding molecule of any one of claims 13 to 15, which further comprises a third polypeptide chain comprising a third component of the first ABS.

19. The binding molecule of any one of claims 1 to 18, which is a multispecific binding molecule comprising a second ABS.

20. The binding molecule of claim 19, wherein (a) the first ABS is a TCE ABS and the second ABS is a TAA ABS or (b) the first ABS is a TAA ABS and the second ABS is a TCE ABS.

21. The binding molecule of claim 20, wherein the first ABS is the TCE ABS.

22. The binding molecule of claim 21, wherein the TCE ABS is capable of binding to CD3, TCRαβ, or TCRγδ.

23. The binding molecule any one of claims 20 to 22, wherein the TAA ABS is capable of binding to:
(a) any TAA identified in Section 6.6; or
(b) AFP, ALK, a BAGE protein, BIRC5 (survivin), BIRC7, β-catenin, brc-abl, BRCA1, BORIS, CA9, carbonic anhydrase IX, caspase-8, CALR, CEACAM5 (also known as carcinoembryonic antigen or CEA), CCR5, CD19, CD20 (MS4A1), CD22, CD30, CD40, CDK4, CEA, CTLA4, cyclin-B1, CYP1B1, EGFR, EGFRvIII, ErbB2/Her2, ErbB3, ErbB4, ETV6-AML, EpCAM, EphA2, Fra-1, FOLR1, a GAGE protein (e.g., GAGE-1 or -2), GD2, GD3, GloboH, glypican-3, GM3, gp100, Her2, HLA/B-raf, HLA/k-ras, HLA/MAGE-A3, hTERT, LMP2, MAGE proteins (e.g., MAGE-1, -2, -3, -4, -6, and -12), MART-1, mesothelin, ML-IAP, Muc1, Muc2, Muc3, Muc4, Muc5, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, Ras, RGS5, Rho, SART-1, SART-3, STEAP1, STEAP2, TAG-72, TGF-β, TMPRSS2, Thompson-nouvelle antigen (Tn), TRP-1, TRP-2, tyrosinase, or uroplakin-3.

24. The binding molecule of any one of claims 1 to 23, wherein the first Fc domain and the second Fc domain associate to form a Fc heterodimer.

25. The binding molecule of claim 24, wherein the Fc heterodimer comprises knob-in-hole mutations, e.g., wherein (i) the first Fc domain comprises one or more knob mutations and the second Fc domain comprises one or more hole mutations or (ii) the first Fc domain comprises one or more hole mutations and the second Fc domain comprises one or more knob mutations.

26. The binding molecule of any one of claims 1 to 25, wherein the first Fc domain and/or the second Fc domain comprise a star mutation.

27. The binding molecule of any one of claims 1 to 26, wherein a tandem Fab is produced following protease cleavage of the PCL.

28. The binding molecule of any one of claims 1 to 26, wherein a multispecific binding molecule comprising the first Fc domain and the second Fc domain is produced following protease cleavage of the PCL.

29. The binding molecule of any one of claims 1 to 28, wherein the PCL comprises a substrate sequence cleavable by any protease set forth in Table A.

30. The binding molecule of any one of claims 1 to 29, wherein the PCL comprises one or more substrate sequences selected from the substrate sequences set forth in Table B.

31. The binding molecule of any one of claims 1 to 30, wherein the PCL comprises the amino acid sequence of any of the PCL sequences selected from the sequences set forth in Table D.

32. The binding molecule of any one of claims 1 to 31, wherein the binding molecule has the configuration depicted in FIG. 3A.

33. The binding molecule of any one of claims 1 to 31, wherein the binding molecule has the configuration depicted in FIG. 3B.

34. The binding molecule of any one of claims 1 to 31, wherein the binding molecule has the configuration depicted in FIG. 3C.

35. The binding molecule of any one of claims 1 to 31, wherein the binding molecule has the configuration depicted in FIG. 3D.

36. The binding molecule of any one of claims 1 to 31, wherein the binding molecule has the configuration depicted in FIG. 3E.

37. The binding molecule of any one of claims 1 to 31, wherein the binding molecule has the configuration depicted in FIG. 3F.

38. The binding molecule of any one of claims 1 to 31, wherein the binding molecule has the configuration depicted in FIG. 7.

39. The binding molecule of any one of claims 1 to 38, wherein the first Fc domain and/or the second Fc domain have at least about 90% sequence identity to any one of SEQ ID NOs: 335, 336, 337, and 338.

40. The binding molecule of any one of claims 1 to 38, wherein the first Fc domain and/or the second Fc domain have at least about 95% sequence identity to any one of SEQ ID NOs: 335, 336, 337, and 338.

41. The binding molecule of any one of claims 1 to 38, wherein the first Fc domain and/or the second Fc domain have at least about 95% sequence identity to any one of SEQ ID NOs: 335, 336, 337, and 338.

42. The binding molecule of any one of claims 1 to 41, wherein the first Fc domain and/or the second Fc domain comprise one or more amino acid substitutions that reduce effector function (e.g., as described in Section 6.10.1).

43. The binding molecule of any one of claims 1 to 41, wherein the first Fc domain and/or the second Fc domain comprise one or more amino acid substitutions that promote heterodimerization (e.g., as described in Section 6.10.2).

44. A pharmaceutical composition comprising the binding molecule of any one of claims 1 to 43 and an excipient.

45. A method of treating cancer, comprising administering to a subject suffering from cancer an effective amount of the binding molecule of any one of claims 1 to 43 or the pharmaceutical composition of claim 44.

46. The method of claim 45, in which the cancer is associated with expression of an epitope bound by the TAA ABS, e.g., as set forth in Table K.

47. A nucleic acid or plurality of nucleic acids encoding the binding molecule of any one of claims 1 to 43.

48. A cell transfected with one or more expression vectors comprising one or more nucleic acid sequences encoding the binding molecule of any one of claims 1 to 43 under the control of one or more promoters.

49. A method of producing a binding molecule, comprising:
(a) culturing the cell of claim 48 in conditions under which the binding molecule is expressed; and
(b) recovering the binding molecule from the cell culture.

50. The method of claim 49, which further comprises enriching for the binding molecule and/or purifying the binding molecule.


SEQUENCE LISTING (346 sequences)

SEQ ID NO: 1 - protein, 5 aa, synthetic construct
GGGGS

SEQ ID NO: 2 - protein, 40 aa, synthetic construct
DEDEDEDEDEDEDEDERPLALWRSDRDRDRDRDRDRDRDR

SEQ ID NO: 3 - protein, 8 aa, synthetic construct
AARGPAIH

SEQ ID NO: 4 - protein, 8 aa, synthetic construct
AAYHLVSQ

SEQ ID NO: 5 - protein, 8 aa, synthetic construct
AGLGISST

SEQ ID NO: 6 - protein, 8 aa, synthetic construct
AGLGVVER

SEQ ID NO: 7 - protein, 4 aa, synthetic construct
ALAL

SEQ ID NO: 8 - protein, 8 aa, synthetic construct
ALFFSSPP

SEQ ID NO: 9 - protein, 8 aa, synthetic construct
ALFKSSFP

SEQ ID NO: 10 - protein, 7 aa, synthetic construct
ALLLALL

SEQ ID NO: 11 - protein, 8 aa, synthetic construct
AQFVLTEG

SEQ ID NO: 12 - protein, 8 aa, synthetic construct
AQNLLGMV

SEQ ID NO: 13 - protein, 8 aa, synthetic construct
AVGLLAPP

SEQ ID NO: 14 - protein, 4 aa, synthetic construct
DAFK

SEQ ID NO: 15 - protein, 4 aa, synthetic construct
DEVD

SEQ ID NO: 16 - protein, 5 aa, synthetic construct
DEVDP

SEQ ID NO: 17 - protein, 6 aa, synthetic construct
DPRSFL

SEQ ID NO: 18 - protein, 8 aa, synthetic construct
DVAQFVLT

SEQ ID NO: 19 - protein, 4 aa, synthetic construct
DVLK

SEQ ID NO: 20 - protein, 8 aa, synthetic construct
DWLYWPGI

SEQ ID NO: 21 - protein, 7 aa, synthetic construct
EDDDDKA

SEQ ID NO: 22 - protein, 6 aa, synthetic construct
  SITE 3: note = Citrulline
  SITE 5: note = Hypofluorous acid modified tyrosine
EPXGYL

SEQ ID NO: 23 - protein, 8 aa, synthetic construct
EPQALAMS

SEQ ID NO: 24 - protein, 8 aa, synthetic construct
ESLPVVAV

SEQ ID NO: 25 - protein, 8 aa, synthetic construct
ESPAYYTA

SEQ ID NO: 26 - protein, 14 aa, synthetic construct
CPPCPAPGGGPSVF

SEQ ID NO: 27 - protein, 10 aa, synthetic construct
FPRPLGITGL

SEQ ID NO: 28 - protein, 8 aa, synthetic construct
FRLLDWQW

SEQ ID NO: 29 - protein, 4 aa, synthetic construct
GFLG

SEQ ID NO: 30 - protein, 10 aa, synthetic construct
GGAANLVRGG

SEQ ID NO: 31 - protein, 5 aa, synthetic construct
GGGRR

SEQ ID NO: 32 - protein, 8 aa, synthetic construct
GGPRGLPG

SEQ ID NO: 33 - protein, 10 aa, synthetic construct
GGQPSGMWGW

SEQ ID NO: 34 - protein, 7 aa, synthetic construct
GGSIDGR

SEQ ID NO: 35 - protein, 8 aa, synthetic construct
GGWHTGRN

SEQ ID NO: 36 - protein, 5 aa, synthetic construct
GIAGQ

SEQ ID NO: 37 - protein, 6 aa, synthetic construct
GKAFRR

SEQ ID NO: 38 - protein, 8 aa, synthetic construct
GPAGLYAQ

SEQ ID NO: 39 - protein, 8 aa, synthetic construct
GPAGMKGL

SEQ ID NO: 40 - protein, 8 aa, synthetic construct
GPEGLRVG

SEQ ID NO: 41 - protein, 8 aa, synthetic construct
GPLGIAGI

SEQ ID NO: 42 - protein, 7 aa, synthetic construct
GPLGVRG

SEQ ID NO: 43 - protein, 8 aa, synthetic construct
GPQGIAGQ

SEQ ID NO: 44 - protein, 8 aa, synthetic construct
GPQGLLGA

SEQ ID NO: 45 - protein, 6 aa, synthetic construct
GPRSFG

SEQ ID NO: 46 - protein, 7 aa, synthetic construct
GPRSFGL

SEQ ID NO: 47 - protein, 8 aa, synthetic construct
GPSHLVLT

SEQ ID NO: 48 - protein, 10 aa, synthetic construct
GVSQNYPIVG

SEQ ID NO: 49 - protein, 10 aa, synthetic construct
GVVQASCRLA

SEQ ID NO: 50 - protein, 6 aa, synthetic construct
GWEHDG

SEQ ID NO: 51 - protein, 6 aa, synthetic construct
HSSKLQ

SEQ ID NO: 52 - protein, 9 aa, synthetic construct
HSSKLQEDA

SEQ ID NO: 53 - protein, 7 aa, synthetic construct
HSSKLQL

SEQ ID NO: 54 - protein, 8 aa, synthetic construct
HTGRSGAL

SEQ ID NO: 55 - protein, 4 aa, synthetic construct
IDGR

SEQ ID NO: 56 - protein, 4 aa, synthetic construct
IEGR

SEQ ID NO: 57 - protein, 8 aa, synthetic construct
ILPRSPAF

SEQ ID NO: 58 - protein, 8 aa, synthetic construct
IPVSLRSG

SEQ ID NO: 59 - protein, 5 aa, synthetic construct
ISSGL

SEQ ID NO: 60 - protein, 6 aa, synthetic construct
ISSGLL

SEQ ID NO: 61 - protein, 7 aa, synthetic construct
ISSGLLS

SEQ ID NO: 62 - protein, 8 aa, synthetic construct
ISSGLLSS

SEQ ID NO: 63 - protein, 7 aa, synthetic construct
ISSGLSS

SEQ ID NO: 64 - protein, 8 aa, synthetic construct
KGSGDVEG

SEQ ID NO: 65 - protein, 9 aa, synthetic construct
KQEQNPGST

SEQ ID NO: 66 - protein, 8 aa, synthetic construct
KRALGLPG

SEQ ID NO: 67 - protein, 8 aa, synthetic construct
LAAPLGLL

SEQ ID NO: 68 - protein, 9 aa, synthetic construct
LAPLGLQRR

SEQ ID NO: 69 - protein, 8 aa, synthetic construct
LAQKLKSS

SEQ ID NO: 70 - protein, 8 aa, synthetic construct
LAQRLRSS

SEQ ID NO: 71 - protein, 5 aa, synthetic construct
LEATA

SEQ ID NO: 72 - protein, 8 aa, synthetic construct
LKAAPRWA

SEQ ID NO: 73 - protein, 8 aa, synthetic construct
LLAPSHRA

SEQ ID NO: 74 - protein, 8 aa, synthetic construct
LPGGLSPW

SEQ ID NO: 75 - protein, 8 aa, synthetic construct
LSGRSANI

SEQ ID NO: 76 - protein, 8 aa, synthetic construct
LSGRSANP

SEQ ID NO: 77 - protein, 8 aa, synthetic construct
LSGRSDDH

SEQ ID NO: 78 - protein, 8 aa, synthetic construct
LSGRSDIH

SEQ ID NO: 79 - protein, 8 aa, synthetic construct
LSGRSDNH

SEQ ID NO: 80 - protein, 8 aa, synthetic construct
LSGRSDNI

SEQ ID NO: 81 - protein, 8 aa, synthetic construct
LSGRSDNP

SEQ ID NO: 82 - protein, 8 aa, synthetic construct
LSGRSDQG

SEQ ID NO: 83 - protein, 8 aa, synthetic construct
LSGRSDQH

SEQ ID NO: 84 - protein, 8 aa, synthetic construct
LSGRSDTH

SEQ ID NO: 85 - protein, 8 aa, synthetic construct
LSGRSDYH

SEQ ID NO: 86 - protein, 8 aa, synthetic construct
LSGRSGNH

SEQ ID NO: 87 - protein, 10 aa, synthetic construct
LVLASSSFGY

SEQ ID NO: 88 - protein, 8 aa, synthetic construct
MDAFLESS

SEQ ID NO: 89 - protein, 8 aa, synthetic construct
MGLFSEAG

SEQ ID NO: 90 - protein, 8 aa, synthetic construct
MIAPVAYR

SEQ ID NO: 91 - protein, 8 aa, synthetic construct
MVLGRSLL

SEQ ID NO: 92 - protein, 12 aa, synthetic construct
NTLSGRSENHSG

SEQ ID NO: 93 - protein, 12 aa, synthetic construct
NTLSGRSGNHGS

SEQ ID NO: 94 - protein, 8 aa, synthetic construct
PAGLWLDP

SEQ ID NO: 95 - protein, 8 aa, synthetic construct
PGGPAGIG

SEQ ID NO: 96 - protein, 5 aa, synthetic construct
  SITE 4: note = Ethyl phenylalanine
PICFF

SEQ ID NO: 97 - protein, 6 aa, synthetic construct
  SITE 4: note = Methyl cysteine
PLGCAG

SEQ ID NO: 98 - protein, 4 aa, synthetic construct
PLGL

SEQ ID NO: 99 - protein, 6 aa, synthetic construct
PLGLAG

SEQ ID NO: 100 - protein, 6 aa, synthetic construct
  VARIANT 6: note = Any amino acid
PLGLAX

SEQ ID NO: 101 - protein, 6 aa, synthetic construct
PLGLWA

SEQ ID NO: 102 - protein, 7 aa, synthetic construct
PLGLWSQ

SEQ ID NO: 103 - protein, 8 aa, synthetic construct
PLTGRSGG

SEQ ID NO: 104 - protein, 5 aa, synthetic construct
PMAKK

SEQ ID NO: 105 - protein, 6 aa, synthetic construct
PPRSFL

SEQ ID NO: 106 - protein, 13 aa, synthetic construct
CPPCPAPGGPSVF

SEQ ID NO: 107 - protein, 8 aa, synthetic construct
PRFRIIGG

SEQ ID NO: 108 - protein, 8 aa, synthetic construct
PVGYTSSL

SEQ ID NO: 109 - protein, 8 aa, synthetic construct
PVQPIGPQ

SEQ ID NO: 110 - protein, 8 aa, synthetic construct
QALAMSAI

SEQ ID NO: 111 - protein, 8 aa, synthetic construct
QGRAITFI

SEQ ID NO: 112 - protein, 8 aa, synthetic construct
QNQALRMA

SEQ ID NO: 113 - protein, 4 aa, synthetic construct
RGPA

SEQ ID NO: 114 - protein, 8 aa, synthetic construct
RGPAFNPM

SEQ ID NO: 115 - protein, 8 aa, synthetic construct
RGPATPIM

SEQ ID NO: 116 - protein, 14 aa, synthetic construct
RKSSIIIRMRDVVL

SEQ ID NO: 117 - protein, 7 aa, synthetic construct
RLQLKAC

SEQ ID NO: 118 - protein, 6 aa, synthetic construct
RLQLKL

SEQ ID NO: 119 - protein, 8 aa, synthetic construct
RMHLRSLG

SEQ ID NO: 120 - protein, 8 aa, synthetic construct
RPSPMWAY

SEQ ID NO: 121 - protein, 8 aa, synthetic construct
RQARVVNG

SEQ ID NO: 122 - protein, 8 aa, synthetic construct
SAGFSLPA

SEQ ID NO: 123 - protein, 8 aa, synthetic construct
SAPAVESE

SEQ ID NO: 124 - protein, 8 aa, synthetic construct
SARGPSRW

SEQ ID NO: 125 - protein, 9 aa, synthetic construct
SGEPAYYTA

SEQ ID NO: 126 - protein, 8 aa, synthetic construct
SGGPLGVR

SEQ ID NO: 127 - protein, 10 aa, synthetic construct
SGRIGFLRTA

SEQ ID NO: 128 - protein, 5 aa, synthetic construct
SGRSA

SEQ ID NO: 129 - protein, 9 aa, synthetic construct
SGRSANPRG

SEQ ID NO: 130 - protein, 8 aa, synthetic construct
SMLRSMPL

SEQ ID NO: 131 - protein, 8 aa, synthetic construct
SPLPLRVP

SEQ ID NO: 132 - protein, 8 aa, synthetic construct
SPLTGRSG

SEQ ID NO: 133 - protein, 8 aa, synthetic construct
SPRSIMLA

SEQ ID NO: 134 - protein, 8 aa, synthetic construct
SSRGPAYL

SEQ ID NO: 135 - protein, 9 aa, synthetic construct
SSRHRRALD

SEQ ID NO: 136 - protein, 15 aa, synthetic construct
SSSFDKGKYKKGDDA

SEQ ID NO: 137 - protein, 15 aa, synthetic construct
SSSFDKGKYKRGDDA

SEQ ID NO: 138 - protein, 8 aa, synthetic construct
STFPFGMF

SEQ ID NO: 139 - protein, 8 aa, synthetic construct
TARGPSFK

SEQ ID NO: 140 - protein, 8 aa, synthetic construct
TGRGPSWV

SEQ ID NO: 141 - protein, 8 aa, synthetic construct
TSGRSANP

SEQ ID NO: 142 - protein, 12 aa, synthetic construct
TSTSGRSANPRG

SEQ ID NO: 143 - protein, 8 aa, synthetic construct
VAGRSMRP

SEQ ID NO: 144 - protein, 8 aa, synthetic construct
VAQFVLTE

SEQ ID NO: 145 - protein, 10 aa, synthetic construct
VHMPLGFLGP

SEQ ID NO: 146 - protein, 8 aa, synthetic construct
VPLSLYSG

SEQ ID NO: 147 - protein, 8 aa, synthetic construct
VVPEGRRS

SEQ ID NO: 148 - protein, 8 aa, synthetic construct
WATPRPMR

SEQ ID NO: 149 - protein, 8 aa, synthetic construct
YGAGLGVV

SEQ ID NO: 150 - protein, 5 aa, synthetic construct
GGGGS

SEQ ID NO: 151 - protein, 4 aa, synthetic construct
GGGS

SEQ ID NO: 152 - protein, 12 aa, synthetic construct
CPPCPAPGPSVF

SEQ ID NO: 153 - protein, 25 aa, synthetic construct
GGGGSGGGGSGGGGSGGGGSGGGGS

SEQ ID NO: 154 - protein, 5 aa, synthetic construct
GSGGS

SEQ ID NO: 155 - protein, 10 aa, synthetic construct
GGGGSGGGGS

SEQ ID NO: 156 - protein, 15 aa, synthetic construct
GGGGSGGGGSGGGGS

SEQ ID NO: 157 - protein, 20 aa, synthetic construct
GGGGSGGGGSGGGGSGGGGS

SEQ ID NO: 158 - protein, 15 aa, synthetic construct
GGGKSGGGKSGGGKS

SEQ ID NO: 159 - protein, 15 aa, synthetic construct
GGGKSGGKGSGKGGS

SEQ ID NO: 160 - protein, 4 aa, synthetic construct
GGGS

SEQ ID NO: 161 - protein, 5 aa, synthetic construct
GGGSG

SEQ ID NO: 162 - protein, 15 aa, synthetic construct
GGKGSGGKGSGGKGS

SEQ ID NO: 163 - protein, 13 aa, synthetic construct
GGSGGGGSGGGGS

SEQ ID NO: 164 - protein, 6 aa, synthetic construct
GGSGGS

SEQ ID NO: 165 - protein, 11 aa, synthetic construct
GGSGGSGGSGS

SEQ ID NO: 166 - protein, 5 aa, synthetic construct
GSGGG

SEQ ID NO: 167 - protein, 5 aa, synthetic construct
GSGSG

SEQ ID NO: 168 - protein, 4 aa, synthetic construct
GSSG

SEQ ID NO: 169 - protein, 10 aa, synthetic construct
GSSGGSGGSG

SEQ ID NO: 170 - protein, 11 aa, synthetic construct
GSSGGSGGSGG

SEQ ID NO: 171 - protein, 12 aa, synthetic construct
GSSGGSGGSGGS

SEQ ID NO: 172 - protein, 13 aa, synthetic construct
GSSGGSGGSGGSG

SEQ ID NO: 173 - protein, 16 aa, synthetic construct
GSSGGSGGSGGSGGGS

SEQ ID NO: 174 - protein, 11 aa, synthetic construct
GSSGGSGGSGS

SEQ ID NO: 175 - protein, 5 aa, synthetic construct
GSSGT

SEQ ID NO: 176 - protein, 5 aa, synthetic construct
GSSSG

SEQ ID NO: 177 - protein, 6 aa, synthetic construct
QGQSGQ

SEQ ID NO: 178 - protein, 7 aa, synthetic construct
QGQSGQG

SEQ ID NO: 179 - protein, 6 aa, synthetic construct
QGQSGS

SEQ ID NO: 180 - protein, 4 aa, synthetic construct
QSGQ

SEQ ID NO: 181 - protein, 5 aa, synthetic construct
QSGQG

SEQ ID NO: 182 - protein, 4 aa, synthetic construct
QSGS

SEQ ID NO: 183 - protein, 4 aa, synthetic construct
SGQG

SEQ ID NO: 184 - protein, 35 aa, synthetic construct
GGGISSGLLSGRSDNHGGGISSGLLSGRSDNHGGS

SEQ ID NO: 185 - protein, 70 aa, synthetic construct
GGGISSGLLSGRSDNHGGGISSGLLSGRSDNHGGSGGGISSGLLSGRSDNHGGGISSGLLSGRSDNHGGS

SEQ ID NO: 186 - protein, 35 aa, synthetic construct
GGSGGSIPVSLRSGGGISSGLLSGRSDNHGGSGGS

SEQ ID NO: 187 - protein, 35 aa, synthetic construct
GGSGGSVPLSLYSGGGISSGLLSGRSDNHGGSGGS

SEQ ID NO: 188 - protein, 35 aa, synthetic construct
GGSHPVGLLARGGGHPVGLLARGGGHPVGLLARGS

SEQ ID NO: 189 - protein, 36 aa, synthetic construct
GGSHPVGLLARGGGHPVGLLARGGSGRSAGGSGRSA

SEQ ID NO: 190 - protein, 18 aa, synthetic construct
AVGLLAPPGGLSGRSANI

SEQ ID NO: 191 - protein, 18 aa, synthetic construct
AVGLLAPPGGLSGRSANP

SEQ ID NO: 192 - protein, 18 aa, synthetic construct
AVGLLAPPGGLSGRSDDH

SEQ ID NO: 193 - protein, 18 aa, synthetic construct
AVGLLAPPGGLSGRSDIH

SEQ ID NO: 194 - protein, 18 aa, synthetic construct
AVGLLAPPGGLSGRSDNH

SEQ ID NO: 195 - protein, 18 aa, synthetic construct
AVGLLAPPGGLSGRSDNI

SEQ ID NO: 196 - protein, 18 aa, synthetic construct
AVGLLAPPGGLSGRSDNP

SEQ ID NO: 197 - protein, 18 aa, synthetic construct
AVGLLAPPGGLSGRSDQH

SEQ ID NO: 198 - protein, 18 aa, synthetic construct
AVGLLAPPGGLSGRSDTH

SEQ ID NO: 199 - protein, 18 aa, synthetic construct
AVGLLAPPGGLSGRSDYH

SEQ ID NO: 200 - protein, 22 aa, synthetic construct
AVGLLAPPGGTSTSGRSANPRG

SEQ ID NO: 201 - protein, 17 aa, synthetic construct
AVGLLAPPSGRSANPRG

SEQ ID NO: 202 - protein, 18 aa, synthetic construct
AVGLLAPPTSGRSANPRG

SEQ ID NO: 203 - protein, 30 aa, synthetic construct
GGALFKSSFPGPAGLYAQPLAQKLKSSGGK

SEQ ID NO: 204 - protein, 33 aa, synthetic construct
GGGGSGGGGSGGGGSFVGGTGGGGSGGGGSGGS

SEQ ID NO: 205 - protein, 34 aa, synthetic construct
GGGGSGGGGSGGGGSISSGLLSGRSDNHGGSGGS

SEQ ID NO: 206 - protein, 34 aa, synthetic construct
GGGGSGGGGSGGGGSVPLSLYSGGGSGGSGGSGS

SEQ ID NO: 207 - protein, 35 aa, synthetic construct
GGGGSGGGGSGPLGLWSQGGGGSGGGGSGGGGSGG

SEQ ID NO: 208 - protein, 33 aa, synthetic construct
GGGGSGGGGSKKAAPGGGGSGGGGSGGGGSGGS

SEQ ID NO: 209 - protein, 33 aa, synthetic construct
GGGGSGGGGSKKAAPVNGGGGGSGGGGSGGGGS

SEQ ID NO: 210 - protein, 33 aa, synthetic construct
GGGGSGGGGSPMAKKGGGGSGGGGSGGGGSGGS

SEQ ID NO: 211 - protein, 33 aa, synthetic construct
GGGGSGGGGSPMAKKVNGGGGGSGGGGSGGGGS

SEQ ID NO: 212 - protein, 33 aa, synthetic construct
GGGGSGGGGSQARAKGGGGSGGGGSGGGGSGGS

SEQ ID NO: 213 - protein, 33 aa, synthetic construct
GGGGSGGGGSQARAKVNGGGGGSGGGGSGGGGS

SEQ ID NO: 214 - protein, 33 aa, synthetic construct
GGGGSGGGGSRQARVVNGGGGGSGGGGSGGGGS

SEQ ID NO: 215 - protein, 41 aa, synthetic construct
GGGGSGGGGSRQARVVNGGGGGSVPLSLYSGGGGGSGGGGS

SEQ ID NO: 216 - protein, 36 aa, synthetic construct
GGGGSGGGGSRQARVVNSVPLSLYSGGGGGSGGGGS

SEQ ID NO: 217 - protein, 33 aa, synthetic construct
GGGGSGGGGSVHMPLGFLGPGGGGSGGGGSGGS

SEQ ID NO: 218 - protein, 28 aa, synthetic construct
GGGGSVHMPLGFLGPGRSRGSFPGGGGS

SEQ ID NO: 219 - protein, 33 aa, synthetic construct
GGGGSVHMPLGFLGPPMAKKGGGGSGGGGSGGS

SEQ ID NO: 220 - protein, 32 aa, synthetic construct
GGGGSVHMPLGFLGPRQARVVNGGGGSGGGGS

SEQ ID NO: 221 - protein, 34 aa, synthetic construct
GGGGSVHMPLGFLGPRQARVVNGGGGSGGGGSGG

SEQ ID NO: 222 - protein, 30 aa, synthetic construct
GGPLAQKLKSSALFKSSFPGPAGLYAQGGR

SEQ ID NO: 223 - protein, 19 aa, synthetic construct
GLSGRSDNHGGAVGLLAPP

SEQ ID NO: 224 - protein, 21 aa, synthetic construct
GLSGRSDNHGGVHMPLGFLGP

SEQ ID NO: 225 - protein, 13 aa, synthetic construct
ISSGLLSGRSANI

SEQ ID NO: 226 - protein, 13 aa, synthetic construct
ISSGLLSGRSANP

SEQ ID NO: 227 - protein, 15 aa, synthetic construct
ISSGLLSGRSANPRG

SEQ ID NO: 228 - protein, 13 aa, synthetic construct
ISSGLLSGRSDDH

SEQ ID NO: 229 - protein, 13 aa, synthetic construct
ISSGLLSGRSDIH

SEQ ID NO: 230 - protein, 13 aa, synthetic construct
ISSGLLSGRSDNH

SEQ ID NO: 231 - protein, 13 aa, synthetic construct
ISSGLLSGRSDNI

SEQ ID NO: 232 - protein, 13 aa, synthetic construct
ISSGLLSGRSDNP

SEQ ID NO: 233 - protein, 13 aa, synthetic construct
ISSGLLSGRSDQH

SEQ ID NO: 234 - protein, 13 aa, synthetic construct
ISSGLLSGRSDTH

SEQ ID NO: 235 - protein, 13 aa, synthetic construct
ISSGLLSGRSDYH

SEQ ID NO: 236 - protein, 13 aa, synthetic construct
ISSGLLSGRSGNH

SEQ ID NO: 237 - protein, 22 aa, synthetic construct
ISSGLLSSGGSGGSLSGRSDNH

SEQ ID NO: 238 - protein, 22 aa, synthetic construct
ISSGLLSSGGSGGSLSGRSGNH

SEQ ID NO: 239 - protein, 30 aa, synthetic construct
KGGPGGPAGIGPLAQRLRSSALFKSSFPGR

SEQ ID NO: 240 - protein, 30 aa, synthetic construct
KSGPGGPAGIGALFFSSPPLAQKLKSSGGR

SEQ ID NO: 241 - protein, 18 aa, synthetic construct
LSGRSDNHGGAVGLLAPP

SEQ ID NO: 242 - protein, 22 aa, synthetic construct
LSGRSDNHGGSGGSISSGLLSS

SEQ ID NO: 243 - protein, 22 aa, synthetic construct
LSGRSDNHGGSGGSQNQALRMA

SEQ ID NO: 244 - protein, 20 aa, synthetic construct
LSGRSDNHGGVHMPLGFLGP

SEQ ID NO: 245 - protein, 22 aa, synthetic construct
LSGRSGNHGGSGGSISSGLLSS

SEQ ID NO: 246 - protein, 22 aa, synthetic construct
LSGRSGNHGGSGGSQNQALRMA

SEQ ID NO: 247 - protein, 22 aa, synthetic construct
QNQALRMAGGSGGSLSGRSDNH

SEQ ID NO: 248 - protein, 22 aa, synthetic construct
QNQALRMAGGSGGSLSGRSGNH

SEQ ID NO: 249 - protein, 30 aa, synthetic construct
RGGALFKSSFPLAQKLKSSGPAGLYAQGGK

SEQ ID NO: 250 - protein, 30 aa, synthetic construct
RGGGPAGLYAQPLAQKLKSSALFKSSFPGG

SEQ ID NO: 251 - protein, 30 aa, synthetic construct
SGGFPRSGGSFNPRTFGSKRKRRGSRGGGG

SEQ ID NO: 252 - protein, 30 aa, synthetic construct
SGPLAQKLKSSGPAGLYAQALFKSSFPGSK

SEQ ID NO: 253 - protein, 22 aa, synthetic construct
TSTSGRSANPRGGGAVGLLAPP

SEQ ID NO: 254 - protein, 24 aa, synthetic construct
TSTSGRSANPRGGGVHMPLGFLGP

SEQ ID NO: 255 - protein, 20 aa, synthetic construct
VHMPLGFLGPGGLSGRSDNH

SEQ ID NO: 256 - protein, 24 aa, synthetic construct
VHMPLGFLGPGGTSTSGRSANPRG

SEQ ID NO: 257 - protein, 21 aa, synthetic construct
SGRSAGGGSGRSAGGGSGRSA

SEQ ID NO: 258 - protein, 35 aa, synthetic construct
HPVGLLARGGGHPVGLLARGGGSGRSAGGGSGRSA

SEQ ID NO: 259 - protein, 8 aa, synthetic construct
GPLGVRGK

SEQ ID NO: 260 - protein, 8 aa, synthetic construct
HPVGLLAR

SEQ ID NO: 261 - protein, 8 aa, synthetic construct
VPMSMRGG

SEQ ID NO: 262 - protein, 8 aa, synthetic construct
RPFSMIMG

SEQ ID NO: 263 - protein, 8 aa, synthetic construct
VPLSLTMG

SEQ ID NO: 264 - protein, 8 aa, synthetic construct
IPESLRAG

SEQ ID NO: 265 - protein, 11 aa, synthetic construct
  VARIANT 1..10: note = This region may encompass 1-10 residues
GGGGGGGGGGS

SEQ ID NO: 266 - protein, 11 aa, synthetic construct
  VARIANT 2..11: note = This region may encompass 1-10 residues
SGGGGGGGGGG

SEQ ID NO: 267 - protein, 4 aa, synthetic construct
GGGG

SEQ ID NO: 268 - protein, 5 aa, synthetic construct
GGGGG

SEQ ID NO: 269 - protein, 6 aa, synthetic construct
GGGGGG

SEQ ID NO: 270 - protein, 7 aa, synthetic construct
GGGGGGG

SEQ ID NO: 271 - protein, 8 aa, synthetic construct
GGGGGGGG

SEQ ID NO: 272 - protein, 9 aa, synthetic construct
GGGGGGGGG

SEQ ID NO: 273 - protein, 5 aa, synthetic construct
ADAAP

SEQ ID NO: 274 - protein, 11 aa, synthetic construct
ADAAPTVSIFP

SEQ ID NO: 275 - protein, 12 aa, synthetic construct
ADAAPTVSIFPP

SEQ ID NO: 276 - protein, 6 aa, synthetic construct
AKTTAP

SEQ ID NO: 277 - protein, 13 aa, synthetic construct
AKTTAPSVYPLAP

SEQ ID NO: 278 - protein, 17 aa, synthetic construct
AKTTPKLEEGEFSEARV

SEQ ID NO: 279 - protein, 9 aa, synthetic construct
AKTTPKLGG

SEQ ID NO: 280 - protein, 6 aa, synthetic construct
AKTTPP

SEQ ID NO: 281 - protein, 13 aa, synthetic construct
AKTTPPSVTPLAP

SEQ ID NO: 282 - protein, 6 aa, synthetic construct
ASTKGP

SEQ ID NO: 283 - protein, 26 aa, synthetic construct
ASTKGPSVFPLAPASTKGPSVFPLAP

SEQ ID NO: 284 - protein, 14 aa, synthetic construct
EGKSSGSGSESKST

SEQ ID NO: 285 - protein, 15 aa, synthetic construct
GEGESGEGESGEGES

SEQ ID NO: 286 - protein, 20 aa, synthetic construct
GEGESGEGESGEGESGEGES

SEQ ID NO: 287 - protein, 15 aa, synthetic construct
GEGGSGEGGSGEGGS

SEQ ID NO: 288 - protein, 15 aa, synthetic construct
GENKVEYAPALMALS

SEQ ID NO: 289 - protein, 15 aa, synthetic construct
GGEGSGGEGSGGEGS

SEQ ID NO: 290 - protein, 15 aa, synthetic construct
GGGESGGEGSGEGGS

SEQ ID NO: 291 - protein, 15 aa, synthetic construct
GGGESGGGESGGGES

SEQ ID NO: 292 - protein, 4 aa, synthetic construct
GGSG

SEQ ID NO: 293 - protein, 5 aa, synthetic construct
GGSGG

SEQ ID NO: 294 - protein, 9 aa, synthetic construct
GGSGGGGSG

SEQ ID NO: 295 - protein, 15 aa, synthetic construct
GHEAAAVMQVQYPAS

SEQ ID NO: 296 - protein, 15 aa, synthetic construct
GKGGSGKGGSGKGGS

SEQ ID NO: 297 - protein, 15 aa, synthetic construct
GKGKSGKGKSGKGKS

SEQ ID NO: 298 - protein, 20 aa, synthetic construct
GKGKSGKGKSGKGKSGKGKS

SEQ ID NO: 299 - protein, 15 aa, synthetic construct
GKPGSGKPGSGKPGS

SEQ ID NO: 300 - protein, 21 aa, synthetic construct
GKPGSGKPGSGKPGSGKPSGS

SEQ ID NO: 301 - protein, 15 aa, synthetic construct
GPAKELTPLKEAKVS

SEQ ID NO: 302 - protein, 12 aa, synthetic construct
GSAGSAAGSGEF

SEQ ID NO: 303 - protein, 18 aa, synthetic construct
GSTSGSGKPGSGEGSTKG

SEQ ID NO: 304 - protein, 19 aa, synthetic construct
GTAAAGAGAAGGAAAGAAG

SEQ ID NO: 305 - protein, 19 aa, synthetic construct
GTSGSSGSGSGGSGSGGGG

SEQ ID NO: 306 - protein, 14 aa, synthetic construct
IRPRAIGGSKPRVA

SEQ ID NO: 307 - protein, 18 aa, synthetic construct
KESGSVSSEQLAQFRSLD

SEQ ID NO: 308 - protein, 15 aa, synthetic construct
KTTPKLEEGEFSEAR

SEQ ID NO: 309 - protein, 19 aa, synthetic construct
PRGASKSGSASQTGSAPGS

SEQ ID NO: 310 - protein, 6 aa, synthetic construct
QPKAAP

SEQ ID NO: 311 - protein, 13 aa, synthetic construct
QPKAAPSVTLFPP

SEQ ID NO: 312 - protein, 27 aa, synthetic construct
RADAAAAGGGGSGGGGSGGGGSGGGGS

SEQ ID NO: 313 - protein, 12 aa, synthetic construct
RADAAAAGGPGS

SEQ ID NO: 314 - protein, 6 aa, synthetic construct
RADAAP

SEQ ID NO: 315 - protein, 9 aa, synthetic construct
RADAAPTVS

SEQ ID NO: 316 - protein, 6 aa, synthetic construct
SAKTTP

SEQ ID NO: 317 - protein, 18 aa, synthetic construct
SAKTTPKLEEGEFSEARV

SEQ ID NO: 318 - protein, 10 aa, synthetic construct
SAKTTPKLGG

SEQ ID NO: 319 - protein, 14 aa, synthetic construct
STAGDTHLGGEDFD

SEQ ID NO: 320 - protein, 5 aa, synthetic construct
TVAAP

SEQ ID NO: 321 - protein, 12 aa, synthetic construct
TVAAPSVFIFPP

SEQ ID NO: 322 - protein, 24 aa, synthetic construct
TVAAPSVFIFPPTVAAPSVFIFPP

SEQ ID NO: 323 - protein, 231 aa, synthetic construct
DKRVESKYGPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 324 - protein, 235 aa, synthetic construct
DKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 325 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 326 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 327 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 328 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 329 - protein, 4 aa, synthetic construct
CPPC

SEQ ID NO: 330 - protein, 4 aa, Homo sapiens
CPSC

SEQ ID NO: 331 - protein, 20 aa, synthetic construct
EPKSCDKTHTCPPCPAPPVA

SEQ ID NO: 332 - protein, 17 aa, synthetic construct
ESKYGPPCPPCPAPPVA

SEQ ID NO: 333 - protein, 15 aa, synthetic construct
CPPCPAPGGGGPSVF

SEQ ID NO: 334 - protein, 4 aa, Homo sapiens
CPPC

SEQ ID NO: 335 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 336 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 337 - protein, 279 aa, synthetic construct
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 338 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 339 - protein, 20 aa, synthetic construct
GGGISSGLLSGRSDNHGGGS

SEQ ID NO: 340 - protein, 15 aa, synthetic construct
GGGHPVGLLARGGGS

SEQ ID NO: 341 - protein, 36 aa, synthetic construct
GGGSGGGSGGGGISSGLLSGRSDNHGGGSGGGSGGS

SEQ ID NO: 342 - protein, 36 aa, synthetic construct
GGGGISSGLLSGRSDNHGGGISSGLLSGRSDNHGGS

SEQ ID NO: 343 - protein, 36 aa, synthetic construct
GGGSGGSIPVSLRSGGGISSGLLSGRSDNHGGSGGS

SEQ ID NO: 344 - protein, 36 aa, synthetic construct
GGGSGGSVPLSLYSGGGISSGLLSGRSDNHGGSGGS

SEQ ID NO: 345 - protein, 36 aa, synthetic construct
GGGSHPVGLLARGGGHPVGLLARGGGHPVGLLARGS

SEQ ID NO: 346 - protein, 36 aa, synthetic construct
GGGSHPVGLLARGGGHPVGLLARGGSGRSAGGSGRS